(19) United States
(12) Patent Application Publication
(10) Pub. No.: US 2023/0266331 A1
(43) Pub. Date: Aug. 24, 2023

(54) USE OF ANTIGEN COMBINATION FOR DETECTING AUTOANTIBODIES IN LUNG CANCER

(71) Applicant: ONCIMMUNE LIMITED, Nottingham (GB)

(72) Inventors: Andrea MURRAY, Ashbourne Derbyshire (GB); Jared ALLEN, Notthinghamshire (GB); Philip GUNNING, Notthinghamshire (GB); Isabel MACDONALD, Notthinghamshire (GB); Celine PARSY-KOWALSKA, Nottinghamshire (GB)

(21) Appl. No.: 18/005,568

(22) PCT Filed: Jul. 14, 2021
(86) PCT No.: PCT/EP2021/069668

§ 371 Date: Jan. 13, 2023

(30) Foreign Application Priority Data
Jul. 14, 2020 (CN) 20210677754.6
Nov. 4, 2020 (GB) 2017434.8

(51) Int. Cl. G01N 33/574

(52) U.S. Cl. CPC G01N 33/57488; G01N 33/57423; G01N 2333/4748; G01N 2800/52

(57) ABSTRACT

The present invention relates generally to the field of antibody detection, and in particular relates to methods involving the detection of autoantibodies relating to lung cancer in a sample comprising patient bodily fluid. In particular, the present invention relates to a method of detecting lung cancer in a mammalian subject by detecting three or more autoantibodies in a test sample, wherein three of the autoantibodies are immunologically specific for the tumour marker antigens p53, SSX1, and either p62 or KOC. The invention also relates to in vitro methods of determining an autoantibody profile, methods of diagnosing and treating lung cancer, methods of predicting response to a lung cancer treatment, use of a panel of three or more tumour marker antigens for the detection of lung cancer, and kits for the detection of autoantibodies.

### FIELD OF THE INVENTION

The present invention relates generally to the field of antibody detection, and in particular relates to methods involving the detection of autoantibodies relating to lung cancer in a sample comprising patient bodily fluid.

### BACKGROUND OF THE INVENTION

Many diagnostic, prognostic and/or monitoring assays rely on detection of a biological marker of a particular disease state or disease susceptibility. Such biological markers are commonly proteins or polypeptides that are characteristic of a particular disease or associated with susceptibility to disease and are often used for the detection of cancers, including lung cancer.

Lung cancer is the most common cancer worldwide, and the most common cause of death from cancer with 1.76 million deaths worldwide recorded in 2018 (WHO fact sheet-https://www.who.int/news-room/fact-sheets/detail/cancer). Lung cancer tends to be diagnosed when symptoms become apparent at which time the tumour is often at an advanced stage (III or IV). Due to this, over 50% of all patients die within 12 months of diagnosis. Early diagnosis more than triples the 5-year survival rate to 56% if the tumour is found to be localised, but unfortunately, only 16% of lung cancers are diagnosed at the localised stage.

Antibodies, and in particular autoantibodies, can serve as biological markers of disease or disease susceptibility. Autoantibodies are naturally occurring antibodies directed to an antigen which an individual's immune system recognises as foreign even though that antigen actually originated in the individual. They may be present in the circulation as circulating free autoantibodies or in the form of circulating immune complexes consisting of autoantibodies bound to their target protein. Differences between a wild type protein expressed by “normal” cells and an altered form of the protein produced by a diseased cell or during a disease process may, in some instances, lead to the altered protein being recognised by an individual's immune system as “non-self” and thus eliciting an immune response in that individual. This may be a humoral B cell-mediated) immune response leading to the production of autoantibodies immunologically specific for the altered protein.

WO 99/58978 describes methods for use in the detection/diagnosis of cancer which are based on evaluating the immune response of an individual to two or more distinct tumour markers. These methods generally involve contacting a sample of bodily fluid taken from the individual with a panel of two or more distinct tumour marker antigens, each derived from a separate tumour marker protein, and detecting the formation of complexes of the tumour marker antigens bound to circulating autoantibodies immunologically specific for the tumour marker proteins. The presence of such circulating autoantibodies is taken as an indication of the presence of cancer.

Assays which measure the immune response of the individual to the presence of tumour marker protein in terms of autoantibody production provide an alternative to the direct measurement or detection of tumour marker protein in bodily fluids. Such assays essentially constitute indirect detection of the presence of tumour marker protein. The nature of the immune response means it is likely that autoantibodies can be elicited by a very small amount of circulating tumour marker protein and indirect methods which rely on detecting the immune response to tumour markers will consequently be more sensitive than methods for the direct measurement of tumour markers in bodily fluids. Assay methods based on the detection of autoantibodies may therefore be of particular value early in the disease process and possibly also in relation to screening of asymptomatic patients, for example in screening to identify individuals “at risk” of developing disease amongst a population of asymptomatic individuals. In addition, methods based on the detection of autoantibodies may be of particular value early in the disease process and may also be used to identify individuals who have developed a disease amongst a population of symptomatic individuals.

A diagnostic test for the early detection of lung cancer has been developed and is commercially available in a number of territories. The test (EarlyCDT Lung; manufactured by Oncimmune Limited, Nottingham, UK) consisting of a panel of 7 tumour marker antigens (p53, SOX2, NY-ESO-1, GBU4-5, CAGE, MAGE A4 and HuD) has been validated (Chapman et al., 2012, Tumor Biol, 33: 1319-1326). The test has undergone what is believed to be the largest randomised controlled trail for the early detection of lung cancer using biomarkers. The successful National Health Service (NHS) ECLS trial of 12,209 high risk smokers in Scotland demonstrated EarlyCDT Lung reduced the incidence of patients with late-stage lung cancer or unclassified presentation at diagnosis, compared to standard clinical practice.

Another diagnostic test that utilises a panel of seven tumour marker antigens (p53, GAGE7, PGP95, CAGE, MAGE-A1, SOX2, GBU4-5) has been developed specifically for detection of lung cancer in a population of Chinese ethnicity (Ren et al., 2017, Oncoimmunology, 7(2)) and is available on the market in China (Seven Kinds of Autoantibodies Test Kit (ELISA); manufactured by Hangzhou Cancer Probe Biotechnology Company, Hangzhou, China (“CancerProbe”)).

However, there is still a requirement for a diagnostic test with improved sensitivity and specificity in order to improve early detection of lung cancer in different ethnic populations, and so a search for new panels of tumour marker antigens was undertaken.

### SUMMARY OF INVENTION

The present application describes new panels of tumour marker antigens that can be used to detect autoantibodies associated with lung cancer. Surprisingly it has been found that a core panel of three tumour marker antigens contributes to the majority of the performance of tests based on these new panels of antigens. The addition of various other tumour marker antigens enhances the performance, especially when targeting populations of different ethnicities. Through the detection of autoantibodies directed to these novel panels of tumour marker antigens, the inventors have devised effective and non-invasive screening methods for lung cancer, and a corresponding kit.

The inventors of the present application have screened a group of tumour marker antigens, and developed a panel of antigen markers suitable for relatively accurate prediction of lung cancer. The inventors have surprisingly found that panels of three or more tumour marker antigens comprising p53, SSX1, and either p62 or KOC, afford improved performance in the detection of lung cancer over the existing diagnostic tests based upon detecting autoantibodies in a human sample.

According to a first aspect, the present invention provides a method of detecting lung cancer in a mammalian subject by detecting three or more autoantibodies in a test sample comprising a bodily fluid from the mammalian subject, wherein three of the autoantibodies are immunologically specific for the tumour marker antigens p53, SSX1, and either p62 or KOC, and wherein the method comprises the steps of:

(a) contacting the test sample with a panel of three or more tumour marker antigens of which three of the tumour marker antigens are p53, SSX1, and either p62 or KOC; and
(b) determining the presence or absence of complexes of the tumour marker antigens bound to autoantibodies present in the test sample,
wherein the presence of complexes containing at least p53, SSX1, and either p62 or KOC is indicative of the presence of lung cancer.

In certain embodiments of the first aspect, the panel of three or more tumour marker antigens comprises p53, SSX1, and p62. In certain alternative embodiments of first aspect, the panel of three or more tumour marker antigens comprises p53, SSX1, and KOC.

In certain embodiments, the panel of three or more tumour marker antigens comprises p53, SSX1, and p62 and/or KOC and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1.

In certain preferred embodiments, the one or more tumour marker antigens is HuD. In certain preferred embodiments, the one or more tumour marker antigens is MAGE A4. In other preferred embodiments, the one or more tumour markers antigens is SOX2. In other preferred embodiments, the one or more tumour markers antigens is CAGE. In other preferred embodiments, the one or more tumour markers antigens is NY-ESO-1.

In certain embodiments, four or more autoantibodies are detected, the method comprises the step of (a) contacting the test sample with a panel of four or more tumour marker antigens of which four of the tumour marker antigens are p53, SSX1, p62 or KOC, and HuD, and wherein the presence of complexes containing at least p53, SSX1, p62 or KOC, and HuD is indicative of the presence of lung cancer. The panel may comprise one of more further tumour marker antigens selected from the group consisting of MAGE A4, SOX2, NY-ESO-1, CAGE, CK20, GBU4-5, p53-95, p53-C, CK8, KRAS, ALDH1, p16, Lmyc2, and α-enolase-1.

In certain embodiments, four or more autoantibodies are detected, the method comprises the step of (a) contacting the test sample with a panel of four or more tumour marker antigens of which four of the tumour marker antigens are p53, SSX1, p62 or KOC, and MAGE A4, and wherein the presence of complexes containing at least p53, SSX1, p62 or KOC, and MAGE A4 is indicative of the presence of lung cancer. The panel may comprise one or more further tumour marker antigens selected from the group consisting of HuD, SOX2, NY-ESO-1, CAGE, CK20, GBU4-5, p53-95, p53-C, CK8, KRAS, ALDH1, p16, Lmyc2, and α-enolase-1.

In certain embodiments, four or more autoantibodies are detected, the method comprises the step of (a) contacting the test sample with a panel of four or more tumour marker antigens of which four of the tumour marker antigens are p53, SSX1, p62 or KOC, and SOX2, and wherein the presence of complexes containing at least p53, SSX1, p62 or KOC, and SOX2 is indicative of the presence of lung cancer. The panel may comprise one of more further tumour marker antigens selected from the group consisting of HuD, MAGE A4, NY-ESO-1, CAGE, CK20, GBU4-5, p53-95, p53-C, CK8, KRAS, ALDH1, p16, Lmyc2, and α-enolase-1.

In certain embodiments, four or more autoantibodies are detected, the method comprises the step of (a) contacting the test sample with a panel of four or more tumour marker antigens of which four of the tumour marker antigens are p53, SSX1, p62 or KOC, and CAGE, and wherein the presence of complexes containing at least p53, SSX1, p62 or KOC, and CAGE is indicative of the presence of lung cancer. The panel may comprise one of more further tumour marker antigens selected from the group consisting of HuD, MAGE A4, SOX2, NY-ESO-1, CK20, GBU4-5, p53-95, p53-C, CK8, KRAS, ALDH1, p16, Lmyc2, and α-enolase-1.

In certain embodiments, four or more autoantibodies are detected, the method comprises the step of (a) contacting the test sample with a panel of four or more tumour marker antigens of which four of the tumour marker antigens are p53, SSX1, p62 or KOC, and NY-ESO-1, and wherein the presence of complexes containing at least p53, SSX1, p62 or KOC, and NY-ESO-1 is indicative of the presence of lung cancer. The panel may comprise one of more further tumour marker antigens selected from the group consisting of HuD, MAGE A4, SOX2, CAGE, CK20, GBU4-5, p53-95, p53-C, CK8, KRAS, ALDH1, p16, Lmyc2, and α-enolase-1.

In certain embodiments, five or more autoantibodies are detected, wherein the method comprises the step of (a) contacting the test sample with a panel of five or more tumour marker antigens of which five of the tumour marker antigens are p53, SSX1, p62 or KOC, HuD and MAGE A4, and wherein the presence of complexes containing at least p53, SSX1, p62 or KOC, HuD and MAGE A4 is indicative of the presence of lung cancer.

In certain embodiments, the panel of five or more tumour marker antigens comprises p53, SSX1, p62 and/or KOC, HuD, and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, SSX1, p62, SSX1, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62. The present inventors have identified that p62 and KOC are structurally similar and share as much as 65% sequence homology. As shown in the experimental data, assays using panels comprising p53, SSX1, and p62, and panels comprising p53, SSX1, and KOC demonstrate superior sensitivity and specificity.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, NY-ESO-1, p16, p53-95, and p53-C and the presence of complexes containing at least p53, SSX1, p62 or KOC, CAGE, NY-ESO-1, p16, p53-95, and p53-C is indicative of the presence of lung cancer.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, NY-ESO-1, SOX2, α-enolase-1, and p53-C and the presence of complexes containing at least p53, SSX1, p62 or KOC, NY-ESO-1, SOX2, α-enolase-1, and p53-C is indicative of the presence of lung cancer.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, HuD, MAGE A4, SOX-2 and CAGE and the presence of complexes containing at least p53, SSX1, p62 or KOC, HuD, MAGE A4, SOX-2 and CAGE is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, HuD, NY-ESO-1, GBU4-5, and p53-C and the presence of complexes containing at least p53, SSX1, p62 or KOC, CAGE, HuD, NY-ESO-1, GBU4-5, and p53-C is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, NY-ESO-1, SOX2, ALDH1, p16, and p53-95 and the presence of complexes containing at least p53, SSX1, p62 or KOC, NY-ESO-1, SOX2, ALDH1, p16, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62, KOC, CAGE, SOX2, α-enolase-1, and p53-C and the presence of complexes containing at least p53, SSX1, p62, KOC, CAGE, SOX2, α-enolase-1, and p53-C is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, HuD, p16, GBU4-5, and p53-95 and the presence of complexes containing at least p53, SSX1, KOC or p62, CAGE, HuD, p16, GBU4-5, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CK20, and the presence of complexes containing at least p53, SSX1, p62 or KOC, HuD, MAGE A4, SOX2, NY-ESO-1 and CK20 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CAGE and the presence of complexes containing at least p53, SSX1, p62 or KOC, HuD, MAGE A4, SOX2, NY-ESO-1 and CAGE is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, CAGE and CK20, and the presence of complexes containing at least p53, SSX1, p62 or KOC, HuD, MAGE A4, SOX2, CAGE and CK20 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, NY-ESO-1, CAGE and CK20, and the presence of complexes containing at least p53, SSX1, p62 or KOC, HuD, MAGE A4, NY-ESO-1, CAGE and CK20 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, ALDH1, and p53-95 and the presence of complexes containing at least p53, SSX1, p62 or KOC, CAGE, HuD, NY-ESO-1, SOX2, ALDH1, p53-95 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, p16, and p53-C and the presence of complexes containing at least p53, SSX1, p62 or KOC, CAGE, HuD, NY-ESO-1, SOX2, p16 and p53-C is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62, KOC, HuD, NY-ESO-1, SOX2, α-enolase-1, and p53-C and the presence of complexes containing at least p53, SSX1, p62, KOC, HuD, NY-ESO-1, SOX2, α-enolase-1, and p53-C is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, KOC or p62, HuD, NY-ESO-1, SOX2, p16, GBU4-5, and p53-95 and the presence of complexes containing at least p53, SSX1, KOC or p62, HuD, NY-ESO-1, SOX2, p16, GBU4-5, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, SOX2, ALDH1, GBU4-5, and Lmyc2, and the presence of complexes containing at least p53, SSX1, KOC or p62, CAGE, SOX2, ALDH1, GBU4-5, and Lmyc2 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, NY-ESO-1, CAGE and CK20, and the presence of complexes containing at least p53, SSX1, p62 or KOC, HuD, MAGE A4, SOX2, NY-ESO-1, CAGE and CK20 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, CK20, CK8 and KRAS, and the presence of complexes containing at least p53, SSX1, p62 or KOC, HuD, MAGE A4, SOX2, CK20, CK8 and KRAS is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62, KOC, CAGE, HuD, NY-ESO-1, p16, and GBU4-5, and the presence of complexes containing at least p53, SSX1, p62, KOC, CAGE, HuD, NY-ESO-1, p16, and GBU4-5 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, KOC or p62, CAGE, HuD, NY-ESO-1, SOX2, ALDH1, and p16, and the presence of complexes containing at least p53, SSX1, KOC or p62, CAGE, HuD, NY-ESO-1, SOX2, ALDH1, and p16 is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, KOC or p62, CAGE, HuD, SOX2, GBU4-5, α-enolase-1, Lmyc2, and p53-C and the presence of complexes containing at least p53, SSX1, KOC or p62, CAGE, HuD, SOX2, GBU4-5, α-enolase-1, Lmyc2, and p53-C is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, p62 or KOC, CAGE, HuD, NY-ESO-1, SOX2, ALDH1, p16, and p53-C and the presence of complexes containing at least p53, SSX1, p62 or KOC, CAGE, HuD, NY-ESO-1, SOX2, ALDH1, p16, and p53-C is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, p62, KOC, CAGE, HuD, NY-ESO-1, SOX2, p16, and α-enolase-1, and the presence of complexes containing at least p53, SSX1, p62, KOC, CAGE, HuD, NY-ESO-1, SOX2, p16, and α-enolase-1 is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C and the presence of complexes containing at least p53, SSX1, p62 or KOC, CAGE, HuD, NY-ESO-1, ALDH1, p16, α-enolase-1, Lmyc2, and p53-C is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS, and the presence of complexes containing at least p53, SSX1, p62 or KOC, HuD, MAGE A4, SOX2, NY-ESO-1, CK20, CK8, p53-95 and KRAS is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS, and the presence of complexes containing at least p53, SSX1, p62 or KOC, HuD, MAGE A4, SOX2, NY-ESO-1, CAGE, CK20, CK8 and KRAS is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS, and the presence of complexes containing at least p53, SSX1, p62 or KOC, HuD, MAGE A4, SOX2, NY-ESO-1, CAGE, GBU4-5, CK8 and KRAS is indicative of the presence of lung cancer.

In certain embodiments, the method further comprises the step of:

(c) detecting the amount of specific binding between the tumour marker antigen and autoantibodies present in the test sample,
wherein the presence or absence of the autoantibody is based upon a comparison between the amount of specific binding observed and a pre-determined cut-off value.

In certain embodiments, the tumour marker antigen is provided in a plurality of different amounts, and wherein the method comprises the steps of:

(a) contacting the test sample with a plurality of different amounts of the tumour marker antigen;
(b) determining the presence or absence of complexes of the tumour marker antigen bound to autoantibodies present in the test sample;
(c) detecting the amount of specific binding between the tumour marker antigen and the autoantibodies;
(d) plotting or calculating a curve of the amount of the specific binding versus the amount of tumour marker antigen for each amount of tumour marker antigen used in step (a); and
(e) determining the presence or absence of the autoantibody based upon the amount of specific binding between the tumour marker antigen and the autoantibody at each different amount of tumour marker antigen used.

In certain embodiments, the method further comprises the steps of:

(d1) calculating a secondary curve parameter from the curve plotted or calculated in step (d); and
(e) determining the presence or absence of the autoantibody based upon a combination of:
(i) the amount of specific binding between the autoantibody and the tumour marker antigen determined in step (b); and
(ii) the secondary curve parameter determined in step (d1).

In a second aspect, the present invention provides an in vitro method of determining an autoantibody profile of an individual suffering from lung cancer by detecting three or more autoantibodies in a test sample comprising a bodily fluid from the mammalian subject, wherein three of the autoantibodies are immunologically specific for the tumour marker antigens p53, SSX1, and either p62 or KOC, which method comprises the steps of:

a) contacting the test sample with a panel of three or more tumour marker antigens of which three of the tumour marker antigens are p53, SSX1, and either p62 or KOC; and
b) determining the presence or absence of complexes of the tumour marker antigens bound to autoantibodies present in the test sample, wherein the method is repeated to build up a profile of autoantibody production.

In a third aspect, the present invention provides a method of diagnosing and treating lung cancer in a mammalian subject by detecting three or more autoantibodies in a test sample comprising a bodily fluid from the mammalian subject, wherein three of the autoantibodies are immunologically specific for the tumour marker antigens p53, SSX1, and either p62 or KOC, which method comprises the steps of:

(a) contacting the test sample with a panel of three or more tumour marker antigens of which three of the tumour marker antigens are p53, SSX1, and either p62 or KOC;
(b) determining the presence or absence of complexes of the tumour marker antigens bound to autoantibodies present in the test sample;
(c) diagnosing the subject with lung cancer when complexes containing at least the tumour marker antigens p53, SSX1, and either p62 or KOC bound to autoantibodies present in the test sample are detected; and
(d) administering a lung cancer treatment to the diagnosed subject.

In a fourth aspect, the present invention provides a method of predicting response to a lung cancer treatment, the method comprising detecting three or more autoantibodies in a test sample comprising a bodily fluid from a mammalian subject, wherein three of the autoantibodies are immunologically specific for the tumour marker antigens p53, SSX1, and either p62 or KOC, which method comprises the steps of:

(a) contacting the test sample with a panel of three or more tumour marker antigens of which three of the tumour marker antigens are p53, SSX1, and either p62 or KOC;
(b) determining the presence or absence of complexes of the tumour marker antigens bound to autoantibodies present in the test sample;
(c) detecting the amount of specific binding between the tumour marker antigens and autoantibodies present in the test sample; and
(d) comparing the amount of specific binding between the tumour marker antigens and the autoantibodies with a previously established relationship between the amount of binding and the likely outcome of treatment,
wherein a change in the amount of specific binding, when compared to controls, predicts that the patient will or will not respond to the lung cancer treatment.

In certain embodiments, the lung cancer treatment is selected from the group consisting of surgery, video-assisted thoracoscopic surgery, radiotherapy, chemotherapy, immunotherapy, radiofrequency ablation, biological therapy, cryotherapy and photodynamic therapy.

In a fifth aspect, the present invention provides use of a panel of three or more tumour marker antigens for the detection of lung cancer in a mammalian subject by detecting autoantibodies immunologically specific for p53, SSX1, and either p62 or KOC in a test sample comprising a bodily fluid from the mammalian subject.

In certain embodiments of the second, third, fourth and fifth aspects, the panel of three or more tumour marker antigens comprises p53, SSX1, and p62. In certain alternative embodiments of the second, third, fourth and fifth aspects, the panel of three or more tumour marker antigens comprises p53, SSX1, and KOC.

In a sixth aspect, the present invention provides a kit for the detection of autoantibodies in a test sample comprising a bodily fluid from a mammalian subject comprising:

(a) a panel of three or more tumour marker antigens of which three of the tumour marker antigens are p53, SSX1, and either p62 or KOC; and
(b) a reagent capable of detecting complexes of the tumour marker antigens bound to autoantibodies present in the test sample.

In certain embodiments of the sixth aspect, the panel of three or more tumour markers antigens comprises p53, SSX1, and p62. In certain alternative embodiments of the sixth aspect, the panel of three or more tumour markers antigens comprises p53, SSX1, and KOC.

In certain embodiments, the kit further comprises:

(c) means for contacting the tumour marker antigens with a test sample comprising a bodily fluid from a mammalian subject.

In certain embodiments, the means for contacting the tumour marker antigens with a test sample comprising a bodily fluid from a mammalian subject comprises the tumour marker antigens immobilised on a chip, slide, plate, wells of a microtitre plate, bead, membrane or nanoparticle.

In certain embodiments, the kit is for the detection of lung cancer.

In all aspects of the invention the tumour marker antigen may be a naturally occurring protein or polypeptide, a recombinant protein or polypeptide, a synthetic protein or polypeptide, a synthetic peptide, a peptide mimetic, a polysaccharide or a nucleic acid.

In all aspects of the invention the bodily fluid may be selected from the group consisting of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears and wound drainage fluid.

In all aspects of the invention the method is preferably carried out in vitro on a test sample comprising a bodily fluid obtained or prepared from the mammalian subject.

In all aspects of the invention the mammalian subject is preferably a human.

In a further aspect of the invention, there is a provided a method of detecting lung cancer in a mammalian subject by detecting an autoantibody in a test sample comprising a bodily fluid from the mammalian subject, wherein the autoantibody is immunologically specific for a tumour marker antigen selected from the group consisting of p53, SSX1, SOX2, GBU4-5, HuD, p53-95 and CK8, and wherein the method comprises the steps of:

(a) contacting the test sample with a tumour marker antigen selected from the group consisting of p53, SSX1, SOX2, GBU4-5, HuD, p53-95 and CK8; and
(b) determining the presence or absence of complexes of the tumour marker antigen bound to autoantibodies present in the test sample,
wherein the presence of said complexes is indicative of the presence of lung cancer.

### BRIEF DESCRIPTION OF THE DRAWINGS

FIG. 1A shows an exemplary plate coating layout. If an antigen is coated at two concentrations (50 and 160 nM) then columns 1, 3, 5, 7 and 9 are 50 nM and columns 2, 4, 6, 8 and 10 are 160 nM.

FIG. 1B shows an exemplary plate dispensing layout. Five to 10 specimens can be run per plate.

FIG. 2 shows a ROC curve for a panel of all 14 markers for Cohort 2 (98 lung cancer cases and 55 benign lung disease controls).

FIG. 3 shows a ROC curve for a nine marker panel of autoantibodies to p53, p62, SSX1, HuD, MAGE A4, SOX2, CK20, NY-ESO-1, and CAGE for Cohort 2 (98 lung cancer cases and 55 benign lung disease controls).

FIG. 4 shows a ROC curve for a five marker panel of autoantibodies to p53, p62, SSX-1, HuD and MAGE A4 for the Cohort 2 (98 lung cancer cases and 55 benign lung disease controls).

FIG. 5 shows a ROC curve for a three marker panel of autoantibodies selected from p53, p62, SSX1 and HuD for Cohort 2 (98 lung cancer cases and 55 benign lung disease controls).

FIG. 6 shows a ROC scatter plot summary of multivariate cut-off solutions obtained using a simulated annealing based algorithm against panels of seven markers for Cohort 3 (148 lung cancer cases and 145 healthy controls).

### DETAILED DESCRIPTION

### A. Definitions

Unless defined otherwise, all technical and scientific terms used herein have the same meaning as commonly understood by the ordinary person skilled in the art to which the invention pertains. Without limiting any term, further clarifications of some of the terms used herein are provided below.

As used herein, the term autoantibody refers to a naturally occurring antibody directed to an antigen which an individual's immune system recognises as foreign even though that antigen actually originated in the individual. In general, autoantibodies include antibodies directed against altered forms of naturally occurring proteins produced by a diseased cell or during a disease process. The altered form of the protein originates in the individual but may be viewed by the individual's immune system as “non-self” and thus elicit an immune response in that individual in the form of autoantibodies immunologically specific to the altered protein. Such altered forms of a protein can include, for example, mutants having altered amino acid sequence, optionally accompanied by changes in secondary, tertiary or quaternary structure, truncated forms, splice variants, altered glycoforms etc. In other embodiments, the autoantibody may be directed to a protein which is overexpressed in a disease state, or as a result of gene amplification or abnormal transcriptional regulation. Overexpression of a protein which is not normally encountered by cells of the immune system in significant amounts can trigger an immune response leading to autoantibody production. In further embodiments the autoantibody may be directed to a foetal form of a protein which becomes expressed in a disease state. If a foetal protein which is normally expressed only in early stages of development, before the immune system is functional, becomes expressed in a disease state, the foetal form expressed in a disease state in the fully developed human may be recognised by the immune system as “foreign”, triggering an immune response leading to autoantibody production. In still further embodiments the autoantibody may be directed against a protein which is expressed at a different location in a disease state. For example, the protein may be expressed at an internal location in healthy individuals but is expressed at a surface exposed location in a disease state such that it is exposed to the circulation and therefore the immune system in the disease state but not in the healthy individual. Herein the protein to which the autoantibody is directed will be referred to as a “tumour marker protein”.

As used herein, the term antigen refers to an immunospecific reagent which complexes with autoantibodies present in the test sample. An antigen is a substance comprising at least one antigenic determinant or epitope capable of interacting specifically with the target autoantibody it is desired to detect, or any capture agent interacting specifically with the variable region or complementary determining regions of said autoantibody. The antigen will typically be a naturally occurring or synthetic biological macromolecule such as, for example, a protein or peptide, a polysaccharide or a nucleic acid and can include antibodies or fragments thereof such as anti-idiotype antibodies. A “tumour marker antigen” is an antigen elevated in subjects with cancer, specifically in this context lung cancer. Herein the terms “tumour marker antigen”, “tumour antigen” and “antigen” will be used interchangeably.

As used herein, the term distinct antigens encompasses antigens derived from different proteins or polypeptides (such as antigens derived from unrelated proteins encoded by different genes).

As used herein, the term antigen variants refers to allelic or other variants of a single antigen, such as a single protein antigen as defined above. Antigen variants will generally be derived from a single gene, and different antigen variants may be expressed in different members of the population or in different disease states. Antigen variants may differ by amino acid sequence or by a post translational modification such as glycosylation, phosphorylation or acetylation. In addition, the term “antigen variant” encompasses antigen mutations such as amino acid substitutions, additions or deletions. Generally an antigen variant will contain less than five (e.g. less than four, less than three, less than two, or one) mutations relative to the wild-type antigen. In certain embodiments of the invention, the antigen may refer to the wild-type antigen. In other embodiments of the invention, the antigen may refer to a variant or mutant version of the antigen. For instance, in certain embodiments, p53 may refer to wild-type p53, or to a variant or mutant version of p53, including but not limited to p53-95 and p53-C.

As used herein, the term bodily fluid when referring to the material to be tested for the presence of autoantibodies using the method of the invention, includes inter alia plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears or wound drainage fluid. As aforesaid, the methods of the invention are preferably carried out in vitro on a test sample comprising bodily fluid removed from the test subject. The type of bodily fluid used may vary depending upon the identity of the autoantibody to be tested and the clinical situation in which the assay is used. In general, it is preferred to perform the assays on samples of serum or plasma. The test sample may include further components in addition to the bodily fluid such as for example diluents, preservatives, stabilising agents, buffers etc. Because the assay method is performed on a sample of bodily fluids it is essentially non-invasive. This means that the assay can be repeated as often as necessary, for example, to build up a profile of the patient's immune response throughout the course of the disease.

As used herein, the terms mammalian subject and subject will be used interchangeably to refer to a subject who is mammalian, preferably human. The subject may have lung cancer. The subject may be suspected of having lung cancer. The subject may have tested positive for lung cancer using ultrasound or surveillance. The subject may have previously been diagnosed with lung cancer and/or be in partial or complete remission. The subject may be undergoing treatment for lung cancer. The subject may be undergoing surgery, video-assisted thoracoscopic surgery, radiotherapy, chemotherapy, immunotherapy, radiofrequency ablation, biological therapy, cryotherapy and/or photodynamic therapy.

### B. Method of Detecting an Autoantibody

The invention provides, in general, an immunoassay method for the detection of autoantibodies immunologically specific for tumour marker proteins associated with lung cancer. The immunoassay method may be used to detect or diagnose lung cancer.

According to a first aspect of the invention there is provided a method of detecting lung cancer in a mammalian subject by detecting three or more autoantibodies in a test sample comprising a bodily fluid from the mammalian subject, wherein three of the autoantibodies are immunologically specific for the tumour marker antigens p53, SSX1, and either p62 or KOC, and wherein the method comprises the steps of:

(a) contacting the test sample with a panel of three or more tumour marker antigens of which three of the tumour marker antigens are p53, SSX1, and either p62 or KOC; and
(b) determining the presence or absence of complexes of the tumour marker antigens bound to autoantibodies present in the test sample, wherein the presence of complexes containing at least p53, SSX1, and either p62 or KOC is indicative of the presence of lung cancer.

In certain embodiments, the method of the invention may further comprise the step of:

(c) detecting the amount of specific binding between the tumour marker antigens and autoantibodies present in the test sample,
wherein the presence or absence of the autoantibody is based upon a comparison between the amount of specific binding observed and a pre-determined cut-off.

Within this embodiment the amount of specific binding between the tumour marker antigens and autoantibodies present in the test sample may be the relative amount of binding or the absolute amount of binding.

Here, the autoantibody may be considered to be present if the amount of specific binding between the tumour marker antigen and autoantibodies present in the test sample is either above or below a pre-determined cut-off. However, generally the autoantibody is considered to be present if the amount of specific binding between the tumour marker antigen and autoantibodies present in the test sample is above a pre-determined cut-off. The pre-determined cut-off may be determined by performing a control assay on known negative samples (e.g. normal individuals) in case-controlled studies. The “normal” individuals will preferably be age-matched controls not having any diagnosis of lung cancer based on clinical, imaging and/or biochemical criteria. In certain embodiments the known negative samples may be derived from individuals with benign lung disease, i.e. those individuals which are at high risk of lung cancer but have not shown any evidence of lung cancer. Preferably the normal individuals do not have any diagnosis of any cancer. Here the amount of specific binding between the tumour marker antigen and autoantibodies present in test samples from normal patients may be detected and averaged to provide a pre-determined cut-off. In certain embodiments the pre-determined cut-off may be determined by selecting the cut-off value giving the largest Youden's value which keeps specificity greater than 90%.

The inventors have surprisingly discovered that a core panel of three tumour marker antigens is particularly effective for the accurate detection and diagnosis of lung cancer. Within the scope of the invention it is contemplated that autoantibodies immunologically specific to a panel of three or more tumour markers antigens may be detected, whereby three of the tumour marker antigens are p53, SSX1, and either p62 or KOC. Within this embodiment a diagnosis of lung cancer may be confirmed based on the presence of complexes of all three tumour marker antigens bound to their respective autoantibodies. The invention also contemplates the detection of autoantibodies which are immunologically specific to a panel of three tumour marker antigens of which the three tumour marker antigens are p53, SSX1, and either p62 or KOC, and the detection of one or more additional autoantibodies immunologically specific for one or more further tumour marker proteins.

As mentioned elsewhere herein, for all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62. The present inventors have identified that p62 and KOC are structurally similar and share as much as 65% sequence homology. As shown in the experimental data, assays using panels comprising p53, SSX1, and p62, and panels comprising p53, SSX1, and KOC demonstrate superior sensitivity and specificity.

In a further embodiment, the invention contemplates that autoantibodies immunologically specific to a panel of four or more tumour markers antigens may be detected, whereby four of the tumour marker antigens are p53, SSX1, p62 or KOC, and HuD. Within this embodiment a diagnosis of lung cancer may be confirmed based on the presence of complexes of all four tumour marker antigens bound to their respective autoantibodies. The invention also contemplates the detection of autoantibodies which are immunologically specific to a panel of four tumour marker antigens of which the four tumour marker antigens are p53, SSX1, p62 or KOC and HuD and the detection of one or more additional autoantibodies immunologically specific for one or more further tumour marker proteins.

In a further embodiment, the invention contemplates that autoantibodies immunologically specific to a panel of four or more tumour markers antigens may be detected, whereby four of the tumour marker antigens are p53, SSX1, p62 or KOC, and MAGE A4. Within this embodiment a diagnosis of lung cancer may be confirmed based on the presence of complexes of all four tumour marker antigens bound to their respective autoantibodies. The invention also contemplates the detection of autoantibodies which are immunologically specific to a panel of four tumour marker antigens of which the four tumour marker antigens are p53, SSX1, p62 or KOC and MAGE A4 and the detection of one or more additional autoantibodies immunologically specific for one or more further tumour marker proteins.

In a further embodiment, the invention contemplates that autoantibodies immunologically specific to a panel of four or more tumour markers antigens may be detected, whereby four of the tumour marker antigens are p53, SSX1, p62 or KOC, and SOX2. Within this embodiment a diagnosis of lung cancer may be confirmed based on the presence of complexes of all four tumour marker antigens bound to their respective autoantibodies. The invention also contemplates the detection of autoantibodies which are immunologically specific to a panel of four tumour marker antigens of which the four tumour marker antigens are p53, SSX1, p62 or KOC and SOX2 and the detection of one or more additional autoantibodies immunologically specific for one or more further tumour marker proteins.

In a further embodiment, the invention contemplates that autoantibodies immunologically specific to a panel of four or more tumour markers antigens may be detected, whereby four of the tumour marker antigens are p53, SSX1, p62 or KOC, and CAGE. Within this embodiment a diagnosis of lung cancer may be confirmed based on the presence of complexes of all four tumour marker antigens bound to their respective autoantibodies. The invention also contemplates the detection of autoantibodies which are immunologically specific to a panel of four tumour marker antigens of which the four tumour marker antigens are p53, SSX1, p62 or KOC and CAGE and the detection of one or more additional autoantibodies immunologically specific for one or more further tumour marker proteins.

In a further embodiment, the invention contemplates that autoantibodies immunologically specific to a panel of four or more tumour markers antigens may be detected, whereby four of the tumour marker antigens are p53, SSX1, p62 or KOC, and NY-ESO-1. Within this embodiment a diagnosis of lung cancer may be confirmed based on the presence of complexes of all four tumour marker antigens bound to their respective autoantibodies. The invention also contemplates the detection of autoantibodies which are immunologically specific to a panel of four tumour marker antigens of which the four tumour marker antigens are p53, SSX1, p62 or KOC and NY-ESO-1 and the detection of one or more additional autoantibodies immunologically specific for one or more further tumour marker proteins.

In a further embodiment, the invention contemplates that autoantibodies immunologically specific to a panel of five or more tumour markers antigens may be detected, whereby five of the tumour marker antigens are p53, SSX1, p62 or KOC, HuD and MAGE A4. Within this embodiment a diagnosis of lung cancer may be confirmed based on the presence of complexes of all five tumour marker antigens bound to their respective autoantibodies. The invention also contemplates the detection of autoantibodies which are immunologically specific to a panel of five tumour marker antigens of which the five tumour marker antigens are p53, SSX1, p62 or KOC, HuD and MAGE A4 and the detection of one or more additional autoantibodies immunologically specific for one or more further tumour marker proteins.

In certain embodiments the mammalian subject may have lung cancer. The subject may have non-small cell lung cancer (NSCLC) such as adenocarcinoma, squamous cell carcinoma, adenosquamous cell carcinoma, large cell carcinoma, or sarcomatoid carcinoma; or the subject may have small cell lung cancer (SCLC).

In other embodiments the mammalian subject may be suspected of having lung cancer. The mammalian subject may have previously tested positive in a lung cancer screen. Here any lung cancer screen is contemplated. In other embodiments the subject may have previously tested positive for lung cancer using ultrasound surveillance or any other imaging method. In certain embodiments, the subject may have previously been diagnosed with lung cancer and/or be in partial or complete remission. The subject may be undergoing treatment for lung cancer. The subject may be undergoing surgery, video-assisted thoracoscopic surgery, radiotherapy, chemotherapy, immunotherapy, radiofrequency ablation, biological therapy, cryotherapy and/or photodynamic therapy.

For the purposes of the invention, subjects which are undergoing treatment for lung cancer or which have previously undergone treatment for lung cancer may still be considered “suspected of having lung cancer”. Herein the treatment for lung cancer may have been performed at any time and the subject may or may not have subsequently been tested for the presence of lung cancer.

The subject may be suspected of having lung cancer due to the presence of a known risk factor for lung cancer. In certain embodiments the subject may be a smoker; the subject may have been exposed to second hand smoke, radon, asbestos, arsenic, diesel exhaust, high air pollution, or other carcinogens; the subject may have received radiation therapy; and/or the subject may have a previous history or family history of lung cancer. Any methods of determining these risk factors are contemplated and the subject may or may not be undergoing or have undergone treatment relevant to the risk factor.

Within the bounds of the present invention the subject may have tested positive in a lung cancer screen at any point prior to performance of the method of the invention. For example, the lung cancer screen may have been performed one hour, two hours, three hours, four hours, five hours, six hours, seven hours, eight hours, nine hours, ten hours, eleven hours, twelve hours, twenty four hours, two days, three days, four days, five days, six days, one week, two weeks, three weeks, four weeks, one month, two months, three months, four months, five months, six months, one year, two years, three years, four years, five years, six years, seven years, eight years, nine years, ten years or more before performance of the method of the invention.

### C. Panels of Tumour Marker Antigens

The present invention provides methods involving the detection of three or more autoantibodies in a test sample comprising a bodily fluid from a mammalian subject, wherein three of the autoantibodies are immunologically specific for the tumour marker antigens p53, SSX1, and either p62 or KOC.

In certain embodiments of the invention the methods may detect three or more autoantibodies, four or more autoantibodies, or five or more autoantibodies. For example, the methods may detect three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight or more autoantibodies.

It is generally accepted that the sensitivity of an assay will be increased by testing for the presence of multiple autoantibodies. Therefore, in some embodiments the methods of the invention contemplate the use of a panel comprising multiple tumour marker antigens, such as three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight or more tumour marker antigens.

For embodiments involving use of panels comprising multiple tumour marker antigens, the methods may require immune complexes containing three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight or more of the antigens to be present for a positive assay result.

These methods may be hereinafter referred to as “panel assays”. Such assays are generally more sensitive than the detection of autoantibodies to a single tumour marker antigen and give a much lower frequency of false negative results (see WO99/58978, WO2004/044590 and WO2006/126008, the contents of which are incorporated herein by reference).

The panel of tumour marker antigens may be tailored having regard to the particular ethnic background of the subject. The inventors have identified a core panel of three tumour marker antigens which can be used to detect associated autoantibodies for the accurate diagnosis of lung cancer in both a Chinese population and a Western population.

In accordance with the core of the invention, the method comprises contacting the test sample with a panel of three or more tumour marker antigens of which three of the tumour marker antigens are p53, SSX1, and either p62 or KOC.

In certain embodiments, the method comprises contacting the test sample with a panel of three or more tumour marker antigens, wherein the panel comprises p53, SSX1, and p62 and/or KOC, and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1. Within this embodiment the panel may comprise three, four, five, six, seven, eight, nine, ten, eleven, twelve, thirteen, fourteen, fifteen, sixteen, seventeen, eighteen, or nineteen of the recited tumour marker antigens.

In certain preferred embodiments, the methods may detect four or more autoantibodies in a test sample comprising a bodily fluid from the mammalian subject, wherein four of the autoantibodies are immunologically specific for the tumour marker antigens are p53, SSX1, p62 or KOC, and HuD. In particularly preferred embodiments, the method comprises contacting the test sample with a panel of four or more tumour marker antigens of which four of the tumour marker antigens are p53, SSX1, p62 or KOC, and HuD. In certain embodiments, the presence of complexes containing at least p53, SSX1, p62 or KOC, and HuD is indicative of the presence of lung cancer.

In certain preferred embodiments, the methods may detect four or more autoantibodies in a test sample comprising a bodily fluid from the mammalian subject, wherein four of the autoantibodies are immunologically specific for the tumour marker antigens are p53, SSX1, p62 or KOC, and MAGE A4. In particularly preferred embodiments, the method comprises contacting the test sample with a panel of four or more tumour marker antigens of which four of the tumour marker antigens are p53, SSX1, p62 or KOC, and MAGE A4. In certain embodiments, the presence of complexes containing at least p53, SSX1, p62 or KOC, and MAGE A4 is indicative of the presence of lung cancer.

In certain preferred embodiments, the methods may detect four or more autoantibodies in a test sample comprising a bodily fluid from the mammalian subject, wherein four of the autoantibodies are immunologically specific for the tumour marker antigens are p53, SSX1, p62 or KOC, and SOX2. In particularly preferred embodiments, the method comprises contacting the test sample with a panel of four or more tumour marker antigens of which four of the tumour marker antigens are p53, SSX1, p62 or KOC, and SOX2. In certain embodiments, the presence of complexes containing at least p53, SSX1, p62 or KOC, and SOX2 is indicative of the presence of lung cancer.

In certain preferred embodiments, the methods may detect four or more autoantibodies in a test sample comprising a bodily fluid from the mammalian subject, wherein four of the autoantibodies are immunologically specific for the tumour marker antigens are p53, SSX1, p62 or KOC, and CAGE. In particularly preferred embodiments, the method comprises contacting the test sample with a panel of four or more tumour marker antigens of which four of the tumour marker antigens are p53, SSX1, p62 or KOC, and CAGE. In certain embodiments, the presence of complexes containing at least p53, SSX1, p62 or KOC, and CAGE is indicative of the presence of lung cancer.

In certain preferred embodiments, the methods may detect four or more autoantibodies in a test sample comprising a bodily fluid from the mammalian subject, wherein four of the autoantibodies are immunologically specific for the tumour marker antigens are p53, SSX1, p62 or KOC, and NY-ESO-1. In particularly preferred embodiments, the method comprises contacting the test sample with a panel of four or more tumour marker antigens of which four of the tumour marker antigens are p53, SSX1, p62 or KOC, and NY-ESO-1. In certain embodiments, the presence of complexes containing at least p53, SSX1, p62 or KOC, and NY-ESO-1 is indicative of the presence of lung cancer.

In certain preferred embodiments, the methods may detect five or more autoantibodies in a test sample comprising a bodily fluid from the mammalian subject, wherein five of the autoantibodies are immunologically specific for the tumour marker antigens are p53, SSX1, p62 or KOC, HuD and MAGE A4. In particularly preferred embodiments, the method comprises contacting the test sample with a panel of five or more tumour marker antigens of which five of the tumour marker antigens are p53, SSX1, p62 or KOC, HuD and MAGE A4. In certain embodiments, the presence of complexes containing at least p53, SSX1, p62 or KOC, HuD and MAGE A4 is indicative of the presence of lung cancer.

In certain embodiments, the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1. Within this embodiment the panel may comprise five, six, seven, eight, nine, ten, eleven or twelve of the recited tumour marker antigens.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, SSX1, p62, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, NY-ESO-1, p16, p53-95, and p53-C and the presence of complexes containing at least p53, SSX1, p62 or KOC, CAGE, NY-ESO-1, p16, p53-95, and p53-C is indicative of the presence of lung cancer.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, NY-ESO-1, SOX2, α-enolase-1, and p53-C and the presence of complexes containing at least p53, SSX1, p62 or KOC, NY-ESO-1, SOX2, α-enolase-1, and p53-C is indicative of the presence of lung cancer.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, HuD, MAGE A4, SOX-2 and CAGE and the presence of complexes containing at least p53, SSX1, p62 or KOC, HuD, MAGE A4, SOX-2 and CAGE is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, HuD, NY-ESO-1, GBU4-5, and p53-C and the presence of complexes containing at least p53, SSX1, p62 or KOC, CAGE, HuD, NY-ESO-1, GBU4-5, and p53-C is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, NY-ESO-1, SOX2, ALDH1, p16, and p53-95 and the presence of complexes containing at least p53, SSX1, p62 or KOC, NY-ESO-1, SOX2, ALDH1, p16, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62, KOC, CAGE, SOX2, α-enolase-1, and p53-C and the presence of complexes containing at least p53, SSX1, p62, KOC, CAGE, SOX2, α-enolase-1, and p53-C is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, HuD, p16, GBU4-5, and p53-95 and the presence of complexes containing at least p53, SSX1, KOC or p62, CAGE, HuD, p16, GBU4-5, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CK20, and the presence of complexes containing at least p53, SSX1, p62 or KOC, HuD, MAGE A4, SOX2, NY-ESO-1 and CK20 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CAGE and the presence of complexes containing at least p53, SSX1, p62 or KOC, HuD, MAGE A4, SOX2, NY-ESO-1 and CAGE is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, CAGE and CK20, and the presence of complexes containing at least p53, SSX1, p62 or KOC, HuD, MAGE A4, SOX2, CAGE and CK20 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, NY-ESO-1, CAGE and CK20, and the presence of complexes containing at least p53, SSX1, p62 or KOC, HuD, MAGE A4, NY-ESO-1, CAGE and CK20 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, ALDH1, and p53-95 and the presence of complexes containing at least p53, SSX1, p62 or KOC, CAGE, HuD, NY-ESO-1, SOX2, ALDH1, and p53-95 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, p16, and p53-C and the presence of complexes containing at least p53, SSX1, p62 or KOC, CAGE, HuD, NY-ESO-1, SOX2, p16, and p53-C is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62, KOC, HuD, NY-ESO-1, SOX2, α-enolase-1, and p53-C and the presence of complexes containing at least p53, SSX1, p62, KOC, HuD, NY-ESO-1, SOX2, α-enolase-1, and p53-C is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, KOC or p62, HuD, NY-ESO-1, SOX2, p16, GBU4-5, and p53-95 and the presence of complexes containing at least p53, SSX1, KOC or p62, HuD, NY-ESO-1, SOX2, p16, GBU4-5, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, SOX2, ALDH1, GBU4-5, and Lmyc2, and the presence of complexes containing at least p53, SSX1, KOC or p62, CAGE, SOX2, ALDH1, GBU4-5, and Lmyc2 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, NY-ESO-1, CAGE and CK20, and the presence of complexes containing at least p53, SSX1, p62 or KOC, HuD, MAGE A4, SOX2, NY-ESO-1, CAGE and CK20 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, CK20, CK8 and KRAS, and the presence of complexes containing at least p53, SSX1, p62 or KOC, HuD, MAGE A4, SOX2, CK20, CK8 and KRAS is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62, KOC, CAGE, HuD, NY-ESO-1, p16, and GBU4-5, and the presence of complexes containing at least p53, SSX1, p62, KOC, CAGE, HuD, NY-ESO-1, p16, and GBU4-5 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, KOC or p62, CAGE, HuD, NY-ESO-1, SOX2, ALDH1, and p16, and the presence of complexes containing at least p53, SSX1, KOC or p62, CAGE, HuD, NY-ESO-1, SOX2, ALDH1, and p16 is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, KOC or p62, CAGE, HuD, SOX2, GBU4-5, α-enolase-1, Lmyc2, and p53-C and the presence of complexes containing at least p53, SSX1, KOC or p62, CAGE, HuD, SOX2, GBU4-5, α-enolase-1, Lmyc2, and p53-C is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, p62 or KOC, CAGE, HuD, NY-ESO-1, SOX2, ALDH1, p16, and p53-C and the presence of complexes containing at least p53, SSX1, p62 or KOC, CAGE, HuD, NY-ESO-1, SOX2, ALDH1, p16, and p53-C is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, p62, KOC, CAGE, HuD, NY-ESO-1, SOX2, p16, and α-enolase-1, and the presence of complexes containing at least p53, SSX1, p62, KOC, CAGE, HuD, NY-ESO-1, SOX2, p16, and α-enolase-1 is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C and the presence of complexes containing at least p53, SSX1, p62 or KOC, CAGE, HuD, NY-ESO-1, ALDH1, p16, α-enolase-1, Lmyc2, and p53-C is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS, and the presence of complexes containing at least p53, SSX1, p62 or KOC, HuD, MAGE A4, SOX2, NY-ESO-1, CK20, CK8, p53-95 and KRAS is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS, and the presence of complexes containing at least p53, SSX1, p62 or KOC, HuD, MAGE A4, SOX2, NY-ESO-1, CAGE, CK20, CK8 and KRAS is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS, and the presence of complexes containing at least p53, SSX1, p62 or KOC, HuD, MAGE A4, SOX2, NY-ESO-1, CAGE, GBU4-5, CK8 and KRAS is indicative of the presence of lung cancer.

The invention also contemplates methods utilising a panel which comprises two or more antigen variants of one or more of the distinct antigens.

Also provided herein is a method of detecting lung cancer in a mammalian subject by detecting an autoantibody in a test sample comprising a bodily fluid from the mammalian subject, wherein the autoantibody is immunologically specific for a tumour marker antigen selected from the group consisting of p53, SSX1, SOX2, GBU4-5, HuD, p53-95 and CK8, and wherein the method comprises the steps of:

(a) contacting the test sample with a tumour marker antigen selected from the group consisting of p53, SSX1, SOX2, GBU4-5, HuD, p53-95 and CK8; and
(b) determining the presence or absence of complexes of the tumour marker antigen bound to autoantibodies present in the test sample,
wherein the presence of said complexes is indicative of the presence of lung cancer.

In certain embodiments, two, three, four, five, six, seven or more autoantibodies are detected, and the method comprises the step of

(a) contacting the test sample with a panel of two or more, three or more, four or more, five or more, six or more or seven or more tumour marker antigens wherein at least two, at least three, at least four, at least five, at least six or seven of the tumour marker antigens are selected from the group consisting of p53, SSX1, SOX2, GBU4-5, HuD, p53-95 and CK8, wherein the presence of complexes containing at least two, at least three, at least four, at least five, at least six or seven of the tumour marker antigens selected from the group consisting of p53, SSX1, SOX2, GBU4-5, HuD, p53-95 and CK8 is indicative of the presence of lung cancer.

In certain embodiments, seven or more autoantibodies are detected, and the method comprises the step of (a) contacting the test sample with a panel of seven or more tumour marker antigens of which seven of the tumour marker antigens are p53, SSX1, SOX2, GBU4-5, HuD, p53-95 and CK8,

wherein the presence of complexes containing at least one, at least two, at least three, at least four, at least five, at least six tumour marker antigens selected from the group consisting of p53, SSX1, SOX2, GBU4-5, HuD, p53-95 and CK8 is indicative of the presence of lung cancer.

In certain embodiments, the presence of complexes containing at least p53, SSX1, SOX2, GBU4-5, HuD, p53-95 and CK8 is indicative of the presence of lung cancer.

### D. Assay Formats

The actual steps of detecting autoantibodies in a sample of bodily fluids may be performed in accordance with immunological assay techniques known per se in the art.

The general features of immunoassays, for example ELISA, radioimmunoassays and the like, are well known to those skilled in the art (see Immunoassay, E. Diamandis and T. Christopoulus, Academic Press, Inc., San Diego, Calif., 1996). Immunoassays for the detection of antibodies having a particular immunological specificity generally require the use of a reagent (antigen) that exhibits specific immunological reactivity with the antibody under test. Depending on the format of the assay this antigen may be immobilised on a solid support. A sample to be tested for the presence of the antibody is brought into contact with the antigen and if antibodies of the required immunological specificity are present in the sample they will immunologically react with the antigen to form antibody-antigen complexes which may then be detected or quantitatively measured.

The methods of the invention may be carried out in any suitable format which enables contact between a test sample suspected of containing the autoantibody and the antigen. Conveniently, contact between the test sample and the antigen may take place in separate reaction chambers such as the wells of a microtitre plate, allowing different antigens or different amounts of antigen to be assayed in parallel, if required. In embodiments in which varying amounts of the antigen are required (see antigen titration method below), these can be coated onto the wells of the microtitre plate by preparing serial dilutions from a stock of antigen across the wells of the microtitre plate. The stock of antigen may be of known or unknown concentration. Aliquots of the test sample may then be added to the wells of the plate, with the volume and dilution of the test sample kept constant in each well. The absolute amounts of antigen added to the wells of the microtitre plate may vary depending on such factors as the nature of the target autoantibody, the nature of the test sample, dilution of the test sample etc. as will be appreciated by those skilled in the art. Generally, the amounts of antigen and the dilution of the test sample will be selected so as to produce a range of signal strengths which fall within the acceptable detection range of the read-out chosen for detection of antigen/autoantibody binding in the method. Conveniently the tested amounts of antigen may vary in the range of from 1.6 nM to 160 mM.

In a further embodiment of the invention the antigen may be immobilised at a discrete location or reaction site on a solid support. In embodiments where different amounts of the antigen are required (see antigen titration method below), these may each be immobilised at discrete locations or reaction sites on a solid support. The entire support may then be brought into contact with the test sample and binding of autoantibody to antigen detected or measured separately at each of the discrete locations or reaction sites. Suitable solid supports include microarrays. Where different amounts of antigen are required, microarrays can be prepared by immobilising different amounts of a particular antigen at discrete, resolvable reaction sites on the array. In other embodiments the actual amount of immobilised antigen molecules may be kept substantially constant but the size of the sites or spots on the array varied in order to alter the amount of binding epitope available, providing a titration series of sites or spots with different amounts of available binding epitope. In such embodiments the two-dimensional surface concentration of the binding epitope(s) on the antigen is important in preparing the titration series, rather than the absolute amount of antigen. Techniques for the preparation and interrogation of protein/peptide microarrays are generally known in the art.

Microarrays may be used to perform multiple assays for autoantibodies of different specificity on a single sample in parallel. This can be done using arrays comprising multiple antigens or sets of antigens.

Certain antigens may comprise or be derived from proteins or polypeptides isolated from natural sources, including but not limited to proteins or polypeptides isolated from patient tissues or bodily fluids (e.g. plasma, serum, whole blood, urine, sweat, lymph, faeces, cerebrospinal fluid, ascites fluid, pleural effusion, seminal fluid, sputum, nipple aspirate, post-operative seroma and wound drainage fluid). In such embodiments the antigen may comprise substantially all of the naturally occurring protein, i.e. protein substantially in the form in which it is isolated from the natural source, or it may comprise a fragment of the naturally occurring protein. To be effective as an antigen in the method of the invention any such fragment must retain immunological reactivity with the autoantibodies for which it will be used to test. Suitable fragments might, for example, be prepared by chemical or enzymatic cleavage of the isolated protein.

In certain embodiments, and depending on the precise nature of the assay in which it will be used, the antigen may comprise a naturally occurring protein, or fragment thereof, linked to one or more further molecules which impart some desirable characteristic not naturally present in the protein. For example, the protein or fragment may be conjugated to a revealing label, such as for example a fluorescent label, coloured label, luminescent label, radiolabel or heavy metal such as colloidal gold. In other embodiments the protein or fragment may be expressed as a recombinantly produced fusion protein. By way of example, fusion proteins may include a tag peptide at the N- or C-terminus to assist in purification of the recombinantly expressed antigen.

Depending on the format of the assay in which it is to be used the antigen may be immobilised on a solid support such as, for example, a chip, slide, wells of a microtitre plate, bead, membrane or nanoparticle. Immobilisation may be effected via non-covalent adsorption, covalent attachment or via tags.

Any suitable attachment means may be used provided this does not adversely affect the ability of the antigen to immunologically react with the target autoantibody to a significant extent.

The invention is not limited to solid phase assays, but also encompasses assays which, in whole or in part, are carried out in liquid phase, for example solution phase bead assays or competition assays.

In one embodiment, antigens may be labelled with a ligand that would facilitate immobilisation, such as biotin. The antigen can then be diluted to a suitable titration range and allowed to react with autoantibodies in patient samples in solution. The resulting immune complexes can then be immobilised on to a solid support via a ligand-receptor interaction (e.g. biotin-streptavidin) and the remainder of the assay performed as described below.

To facilitate the production of biotinylated antigens for use in the assay methods of the invention, cDNAs encoding a full length antigen, a truncated version thereof or an antigenic fragment thereof may be expressed as a fusion protein labelled with a protein or polypeptide tag to which the biotin co-factor may be attached, for example via an enzymatic reaction.

Vectors for the production of recombinant biotinylated antigens are commercially available from a number of sources. Alternatively, biotinylated antigens may be produced by covalent linkage of biotin to the antigen molecule following expression and purification.

As aforesaid, the immunoassay used to detect autoantibodies according to the invention may be based on standard techniques known in the art. In a most preferred embodiment the immunoassay may be an ELISA. ELISAs are generally well known in the art. In a typical indirect ELISA an antigen having specificity for the autoantibodies under test is immobilised on a solid surface (e.g. the wells of a standard microtiter assay plate, or the surface of a microbead or a microarray) and a sample comprising bodily fluid to be tested for the presence of autoantibodies is brought into contact with the immobilised antigen. Any autoantibodies of the desired specificity present in the sample will bind to the immobilised antigen. The bound antigen/autoantibody complexes may then be detected using any suitable method. In one preferred embodiment a labelled secondary anti-human immunoglobulin antibody, which specifically recognises an epitope common to one or more classes of human immunoglobulins, is used to detect the antigen/autoantibody complexes. Typically the secondary antibody will be anti-IgG or anti-IgM. The secondary antibody is usually labelled with a detectable marker, typically an enzyme marker such as, for example, peroxidase or alkaline phosphatase, allowing quantitative detection by the addition of a substrate for the enzyme which generates a detectable product, for example a coloured, chemiluminescent or fluorescent product. Other types of detectable labels known in the art may be used with equivalent effect.

### Antigen Titration Method

In WO2006/126008 (the contents of which are incorporated herein by reference), it was determined that the performance, and more specifically the clinical utility and reliability, of assays based on detection of autoantibodies as biological markers of disease can be improved dramatically by inclusion of an antigen titration step.

By testing the sample suspected of containing antibodies against a series of different amounts of antigen (also referred to herein as a titration series) and constructing a titration curve it is possible to reliably identify true positive screening results independently of the absolute amount of antibody present in the sample. The antigen titration method of WO2006/126008 provides greater specificity and sensitivity than measuring autoantibody reactivity at a single antigen concentration, or methods in which the serum sample is titrated rather than the antigen.

Accordingly, in certain embodiments, the invention contemplates that the tumour marker antigen is provided in a plurality of different amounts, and wherein the method comprises the steps of:

(a) contacting the test sample with a plurality of different amounts of the tumour marker antigen;
(b) determining the presence or absence of complexes of the tumour marker antigen bound to autoantibodies present in the test sample;
(c) detecting the amount of specific binding between the tumour marker antigen and the autoantibodies;
(d) plotting or calculating a curve of the amount of the specific binding versus the amount of tumour marker antigen for each amount of tumour marker antigen used in step (a); and
(e) determining the presence or absence of the autoantibody based upon the amount of specific binding between the tumour marker antigen and the autoantibody at each different amount of tumour marker antigen used.

In practice the different amounts of the tumour marker antigen will generally be provided by altering the concentration of the tumour marker antigen utilised. Therefore, the terms “different amount” and “different concentration” may be used interchangeably. However, within the scope of the invention, any method of altering the amount of tumour marker antigen is contemplated. Skilled readers will appreciate that in the method of the invention the amount of antigenic determinants or epitopes available for binding to the target autoantibody is important for establishing a titration series (i.e. a set of antigens provided in different amounts). In many assay formats the amount of antigenic determinants or epitopes available for binding is directly correlated with the amount of antigen molecules present. However, in other embodiments, such as certain solid phase assay systems, the amount of exposed antigenic determinants or epitopes may not correlate directly with the amount of antigen but may depend on other factors, such as attachment to the solid surface and conformational presentation. In these embodiments, references herein to “different amounts of antigen” in a titration series may be taken to refer to different amounts of the antigenic determinant or epitope. In particular embodiments, variation in the amount of antigen may be achieved by changing the antigen or epitope density against which the sample is tested, or by maintaining antigen or epitope density but increasing the surface area over which antigen is immobilised, or both.

Within this embodiment, a “set of antigens” refers to a single antigen to be tested at different amounts in the method of the invention.

In accordance with the present invention, the method comprises contacting the test sample with a panel of three or more tumour marker antigens of which three of those tumour marker antigens are p53, SSX1, and either p62 or KOC. In such embodiments where multiple antigens are contemplated, a “set of distinct antigens” refers to a single antigen to be tested at different amounts in the method of the invention, wherein each antigen is a “distinct antigen” derived from different proteins or polypeptides (such as antigens derived from unrelated proteins encoded by different genes), as defined above.

A given microarray may include exclusively sets of distinct antigens derived from different proteins or polypeptides, or exclusively sets of distinct antigens derived from different peptide epitopes of a single protein or polypeptide, or a mixture of the two in any proportion.

It should be noted that each individual set of antigens of different amounts in any embodiment of the invention will generally comprise just one antigen and not mixtures thereof.

A set of antigen variants refers to a single antigen variant to be tested at different amounts in the method of the invention.

In certain embodiments, the presence or absence of the autoantibody may be determined based upon the collective values of the amount of specific binding for all of the amounts of tumour marker antigen used. During the methods of the invention the relative or absolute amount of specific binding between autoantibody and the antigen is determined for each different amount of antigen (antigenic determinant or epitope) tested and used to plot or calculate a curve of the (relative or absolute) amount of specific binding versus the amount of antigen for each amount of antigen tested. The presence in the test sample of autoantibody reactive with the antigen used in the assay is determined based upon the amount of specific binding observed at each antigen amount and is generally indicated by a dose-response curve, which is typically S-shaped or sigmoidal. Therefore, in certain embodiments the presence or absence of the autoantibody is determined by screening the plot for the presence of a dose response curve such as a generally S-shaped or sigmoidal curve. If there is no variation in detectable binding over the different amounts of antigen tested then this can be scored as an absence of a detectable amount of the autoantibody.

In certain embodiments, the presence or absence of the autoantibody is determined based upon the collective values of the amount of specific binding for all of the amounts of tumour marker antigen used.

In certain embodiments, the presence or absence of the autoantibody is determined by screening the plot of step (d) for the presence of a dose response curve.

In certain embodiments, the dose response curve is a generally S-shaped or sigmoidal curve.

In one embodiment, the presence or absence of autoantibody is determined by comparison of the amount of specific binding between the autoantibody and the antigen with pre-determined cut-off values. Here, a curve of the amount of specific binding versus the amount of antigen for each amount of antigen used in the titration series is plotted, and the level of binding in known positive samples (e.g. a populations of patients with disease) are compared with the level of binding observed in known negative samples (e.g. normal individuals) in case-controlled studies. Cut-off values for autoantibody binding at one or more points on the titration curve are chosen that maximise sensitivity (few false negatives) while maintaining high specificity (few false positives). Provided the curve of the amount of specific binding versus the amount of antigen for each amount of antigen used in the titration series is a dose response curve, a measurement is considered to be positive if the amount of specific binding determined for one or more points on the titration curve is above the predetermined cut-off point value. In certain embodiments the pre-determined cut-off may be determined by selecting the cut-off value giving the largest Youden's value whilst keeping specificity greater than 90%.

It should be noted that the antigen titration embodiment may be used with all methods of the invention, including methods of detecting lung cancer, methods of diagnosing and treating lung cancer, methods of predicting response to an anti-lung cancer treatment and methods of determining an antibody profile. In addition, antigen titration may be used in embodiments wherein only a single autoantibody is detected as well as in embodiments where a panel of antigens is used to detect multiple autoantibodies.

### Double Cut-Off Method

It is generally accepted that the sensitivity of an assay will be increased by measuring autoantibodies against multiple antigens. However, this increased sensitivity is usually associated with a proportional decrease in specificity and assay methods may therefore be limited in the number of antigens which they can use. In certain embodiments the present method may account for the decrease in specificity by using an antigen titration method which determines the level of specific binding between the autoantibody and the antigen and assessment of a secondary curve parameter, with only test results considered positive when compared to cut-off points for both of these metrics being classified as positive. This method will be referred to herein as the “double cut-off” method and is fully described in WO2015/193678 (the contents of which are incorporated herein by reference).

In certain embodiments the methods of the invention further comprise the steps of:

(d1) calculating a secondary curve parameter from the curve plotted or calculated in step (d); and
(e) determining the presence or absence of the autoantibody based upon a combination of:
(i) the amount of specific binding between the autoantibody and the tumour marker antigen determined in step (b); and
(ii) the secondary curve parameter determined in step (d1).

The double cut-off method utilises the antigen titration methodology described above. Following detection of the amount of antigen/autoantibody binding at each amount of antigen used in the titration series, and the plotting of a curve of the amount of specific binding versus the amount of antigen for each amount of antigen used in the titration series, a secondary curve parameter is calculated. The secondary curve parameter may be calculated from either a linear or logarithmic regression curve. Herein a secondary curve parameter is any calculated value which provides an indication of the nature of the curve. For example, the secondary curve parameter may be Slope, Intercept, AUC, SlopeMax or dissociation constant (Kd).

Accordingly, in certain embodiments the secondary curve parameter is selected from the group consisting of Slope, Intercept, AUC, SlopeMax and dissociation constant (Kd).

In certain embodiments, the secondary curve parameter is calculated from either a linear or logarithmic regression curve.

In certain embodiments the secondary curve parameter may be determined by fitting a logistic curve, such as a 4 parameter logistic curve, to the curve of the amount of specific binding versus the amount of antigen for each amount of antigen used in the titration series. In this embodiment the secondary curve parameter may be Maximum Asymptote, Minimum Asymptote, Hill Slope (or Slope Factor) or Inflection Point.

Accordingly, in certain embodiments the secondary curve parameter is Maximum Asymptote, Minimum Asymptote, Hill Slope (or Slope Factor) or Inflection Point of a logistic curve fitted to each curve plotted or calculated in step (c).

Once a secondary curve parameter has been obtained it will be combined with the antigen/autoantibody binding data in order to determine the presence or absence of the autoantibody. Here, the amount of specific binding between the autoantibody and the antigen will be compared with a predetermined cut-off value as described above.

The cut-off for the secondary curve parameter is determined using known positive samples (e.g. a set of case-control sample sets consisting of a cohort of patients with disease) and known negative samples (e.g. a cohort of normal individuals in case-controlled studies). For each sample a curve of the amount of specific binding versus the amount of antigen for each amount of antigen used in the titration series is plotted, and the secondary curve parameter observed in the known positive sample (e.g. patients with disease) is compared with the secondary curve parameter observed in the known negative sample (e.g. normal individuals). Cut-off values for the secondary curve parameters are chosen that maximise specificity (few false positives) when used in combination with the cut-off for antigen/autoantibody binding discussed above.

Upon calculating the cut-off value for the secondary curve parameter, the directionality required for a positive reading, i.e. whether a value above or below the cut-off is considered positive, is also determined. The directionality required for a positive reading will depend upon the antigen and the secondary curve parameter. A measurement is considered to be ultimately positive, i.e. indicative of the presence of autoantibody in the test sample, if it is both above the cut-off for antigen/autoantibody binding and demonstrates the directionality required for a positive reading compared to the cut-off for the secondary curve parameter.

It should be noted that the double cut-off embodiment may be used with all methods of the invention, including methods of detecting lung cancer, methods of diagnosing and treating lung cancer, methods of predicting response to an anti-lung cancer treatment and methods of determining an antibody profile. In addition, the double cut off method may be used in embodiments wherein only a single autoantibody is detected as well as in embodiments where a panel of antigens is used to detect multiple autoantibodies. In the panel embodiment it should be noted that the secondary curve parameter calculated for each antigen within the panel need not necessarily be the same. However, in some embodiments the secondary curve parameter calculated for each antigen within the panel may be the same.

### E. Applications of the Method

The immunoassay methods according to the invention may be employed in a variety of different clinical situations. In accordance with the invention, the methods are useful for the detection of lung cancer. In particular, the methods may be used in the detection or diagnosis of lung cancer, in screening a population of asymptomatic human subjects in order to diagnose the presence of lung cancer, in the detection of primary or secondary (metastatic) lung cancer, or in screening for early neoplastic or early carcinogenic change in asymptomatic patients.

### Diagnosing and Treating Lung Cancer

In certain embodiments, there is provided a method of diagnosing and treating lung cancer in a mammalian subject by detecting three or more autoantibodies in a test sample comprising a bodily fluid from the mammalian subject, wherein three of the autoantibodies are immunologically specific for the tumour marker antigens p53, SSX1, and either p62 or KOC, which method comprises the steps of:

(a) contacting the test sample with a panel of three or more tumour marker antigens of which three of the tumour marker antigens are p53, SSX1, and either p62 or KOC;
(b) determining the presence or absence of complexes of the tumour marker antigens bound to autoantibodies present in the test sample;
(c) diagnosing the subject with lung cancer when complexes containing at least the tumour marker antigens p53, SSX1, and either p62 or KOC bound to autoantibodies present in the test sample are detected; and
(d) administering a lung cancer treatment to the diagnosed subject.

In certain preferred embodiments of the method, the three tumour marker antigens are p53, SSX1, and p62. In certain preferred alternative embodiments of the method, the three tumour marker antigens are p53, SSX1, and KOC.

Within this aspect, the autoantibody may be considered to be present if the amount of specific binding between the tumour marker antigen and autoantibodies present in the test sample is either above or below a pre-determined cut-off, as explained above.

In certain embodiments, the panel of three or more tumour marker antigens comprises p53, SSX1, and p62 and/or KOC, and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and HuD, and wherein the presence of at least complexes containing p53, SSX1, p62 or KOC, and HuD is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and MAGE A4, and wherein the presence of at least complexes containing p53, SSX1, p62 or KOC, and MAGE A4 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and SOX2, and wherein the presence of at least complexes containing p53, SSX1, p62 or KOC, and SOX2 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and CAGE, and wherein the presence of at least complexes containing p53, SSX1, p62 or KOC, and CAGE is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and NY-ESO-1, and wherein the presence of at least complexes containing p53, SSX1, p62 or KOC, and NY-ESO-1 is detected.

In a particularly preferred embodiment, the method involves detecting five or more autoantibodies and the method comprises the step of (a) contacting the test sample with a panel of five or more tumour marker antigens of which five of the tumour marker antigens are p53, SSX1, p62 or KOC, HuD and MAGE A4, and wherein the presence of at least complexes containing p53, SSX1, p62 or KOC, HuD and MAGE A4 is detected.

In certain embodiments, the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, SSX1, p62, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, NY-ESO-1, p16, p53-95, and p53-C and the presence of complexes containing at least p53, SSX1, p62 or KOC, CAGE, NY-ESO-1, p16, p53-95, and p53-C is indicative of the presence of lung cancer.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, NY-ESO-1, SOX2, α-enolase-1, and p53-C and the presence of complexes containing at least p53, SSX1, p62 or KOC, NY-ESO-1, SOX2, α-enolase-1, and p53-C is indicative of the presence of lung cancer.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, HuD, MAGE A4, SOX-2 and CAGE and the presence of complexes containing at least p53, SSX1, p62 or KOC, HuD, MAGE A4, SOX-2 and CAGE is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, HuD, NY-ESO-1, GBU4-5, and p53-C and the presence of complexes containing at least p53, SSX1, p62 or KOC, CAGE, HuD, NY-ESO-1, GBU4-5, and p53-C is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, NY-ESO-1, SOX2, ALDH1, p16, and p53-95 and the presence of complexes containing at least p53, SSX1, p62 or KOC, NY-ESO-1, SOX2, ALDH1, p16, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62, KOC, CAGE, SOX2, α-enolase-1, and p53-C and the presence of complexes containing at least p53, SSX1, p62, KOC, CAGE, SOX2, α-enolase-1, and p53-C is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, HuD, p16, GBU4-5, and p53-95 and the presence of complexes containing at least p53, SSX1, KOC or p62, CAGE, HuD, p16, GBU4-5, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CK20, and the presence of complexes containing at least p53, SSX1, p62 or KOC, HuD, MAGE A4, SOX2, NY-ESO-1 and CK20 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CAGE and the presence of complexes containing at least p53, SSX1, p62 or KOC, HuD, MAGE A4, SOX2, NY-ESO-1 and CAGE is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, CAGE and CK20, and the presence of complexes containing at least p53, SSX1, p62 or KOC, HuD, MAGE A4, SOX2, CAGE and CK20 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, NY-ESO-1, CAGE and CK20, and the presence of complexes containing at least p53, SSX1, p62 or KOC, HuD, MAGE A4, NY-ESO-1, CAGE and CK20 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, ALDH1, and p53-95 and the presence of complexes containing at least p53, SSX1, p62 or KOC, CAGE, HuD, NY-ESO-1, SOX2, ALDH1, and p53-95 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, p16, and p53-C and the presence of complexes containing at least p53, SSX1, p62 or KOC, CAGE, HuD, NY-ESO-1, SOX2, p16, and p53-C is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62, KOC, HuD, NY-ESO-1, SOX2, α-enolase-1, and p53-C and the presence of complexes containing at least p53, SSX1, p62, KOC, HuD, NY-ESO-1, SOX2, α-enolase-1, and p53-C is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, KOC or p62, HuD, NY-ESO-1, SOX2, p16, GBU4-5, and p53-95 and the presence of complexes containing at least p53, SSX1, KOC or p62, HuD, NY-ESO-1, SOX2, p16, GBU4-5, and p53-95 is indicative of the presence of lung cancer.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, SOX2, ALDH1, GBU4-5, and Lmyc2, and the presence of complexes containing at least p53, SSX1, KOC or p62, CAGE, SOX2, ALDH1, GBU4-5, and Lmyc2 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, NY-ESO-1, CAGE and CK20, and the presence of complexes containing at least p53, SSX1, p62 or KOC, HuD, MAGE A4, SOX2, NY-ESO-1, CAGE and CK20 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, CK20, CK8 and KRAS, and the presence of complexes containing at least p53, SSX1, p62 or KOC, HuD, MAGE A4, SOX2, CK20, CK8 and KRAS is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, p62, KOC, CAGE, HuD, NY-ESO-1, p16, and GBU4-5, and the presence of complexes containing at least p53, SSX1, p62, KOC, CAGE, HuD, NY-ESO-1, p16, and GBU4-5 is indicative of the presence of lung cancer.

In certain embodiments, nine or more autoantibodies are detected, the method comprises the step of (a) contacting the test sample with a panel of nine or more tumour marker antigens of which nine of the tumour marker antigens are p53, SSX1, KOC or p62, CAGE, HuD, NY-ESO-1, SOX2, ALDH1, and p16, and the presence of complexes containing at least p53, SSX1, KOC or p62, CAGE, HuD, NY-ESO-1, SOX2, ALDH1, and p16 is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, KOC or p62, CAGE, HuD, SOX2, GBU4-5, α-enolase-1, Lmyc2, and p53-C and the presence of complexes containing at least p53, SSX1, KOC or p62, CAGE, HuD, SOX2, GBU4-5, α-enolase-1, Lmyc2, and p53-C is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, p62 or KOC, CAGE, HuD, NY-ESO-1, SOX2, ALDH1, p16, and p53-C and the presence of complexes containing at least p53, SSX1, p62 or KOC, CAGE, HuD, NY-ESO-1, SOX2, ALDH1, p16, and p53-C is indicative of the presence of lung cancer.

In certain embodiments, ten or more autoantibodies are detected, the method comprises the step of (a) contacting the test sample with a panel of ten or more tumour marker antigens of which ten of the tumour marker antigens are p53, SSX1, p62, KOC, CAGE, HuD, NY-ESO-1, SOX2, p16, and α-enolase-1, and the presence of complexes containing at least p53, SSX1, p62, KOC, CAGE, HuD, NY-ESO-1, SOX2, p16, and α-enolase-1 is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C and the presence of complexes containing at least p53, SSX1, p62 or KOC, CAGE, HuD, NY-ESO-1, ALDH1, p16, α-enolase-1, Lmyc2, and p53-C is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS, and the presence of complexes containing at least p53, SSX1, p62 or KOC, HuD, MAGE A4, SOX2, NY-ESO-1, CK20, CK8, p53-95 and KRAS is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS, and the presence of complexes containing at least p53, SSX1, p62 or KOC, HuD, MAGE A4, SOX2, NY-ESO-1, CAGE, CK20, CK8 and KRAS is indicative of the presence of lung cancer.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS, and the presence of complexes containing at least p53, SSX1, p62 or KOC, HuD, MAGE A4, SOX2, NY-ESO-1, CAGE, GBU4-5, CK8 and KRAS is indicative of the presence of lung cancer.

It should be noted that the invention is in no way limited to any specific lung cancer treatment. In certain embodiments the lung cancer treatment may be selected from the group consisting of surgery, video-assisted thoracoscopic surgery, radiotherapy, chemotherapy, immunotherapy, radiofrequency ablation, biological therapy, cryotherapy and photodynamic therapy.

Within the bounds of the invention, the lung cancer treatment may be administered at any time following the diagnosis of lung cancer. For example, the lung cancer treatment may be administered one hour, two hours, three hours, four hours, five hours, six hours, seven hours, eight hours, nine hours, ten hours, eleven hours, twelve hours, twenty four hours, two days, three days, four days, five days, six days, one week, two weeks, three weeks, four weeks, one month, two months, three months, four months, five months, six months, one year or more after the diagnosis of lung cancer. Multiple administrations of lung cancer treatment with any spacing between rounds of treatment are also contemplated.

Administration of the lung cancer treatment at a geographical location different from the geographical location at which the lung cancer diagnosis was performed is contemplated. Further, the lung cancer treatment may be administered by a person different from the person performing the diagnosis, irrespective of whether the diagnosis and treatment are performed at the same or different geographical locations.

Within this embodiment of the invention all limitations discussed above in relation to the various methods of the invention are contemplated in relation to the method of diagnosing and treating lung cancer.

### Predicting Response to a Lung Cancer Treatment

In one aspect, the autoantibody detection method of the invention may be used for treatment stratification, i.e. to determine whether a particular subject or group of subjects is more or less likely to respond to a particular lung cancer treatment. The methods may be used in predicting the response of a lung cancer patient to a lung cancer treatment, in the selection of a lung cancer therapy, in the selection of a lung cancer therapy for use in a particular patient, in predicting response to therapy, in predicting survival responsive to treatment, or in predicting the risk of immune-related adverse events (irAEs) in patients undergoing immunotherapy (e.g. treatment with checkpoint inhibitors). The lung cancer therapy or treatment may be, for example, surgery, video-assisted thoracoscopic surgery, radiotherapy, chemotherapy, immunotherapy, radiofrequency ablation, biological therapy, cryotherapy and photodynamic therapy.

The invention therefore provides a method of predicting response to a lung cancer treatment, the method comprising detecting three or more autoantibodies in a test sample comprising a bodily fluid from a mammalian subject, wherein three of the autoantibodies are immunologically specific for the tumour marker antigens p53, SSX1, and either p62 or KOC, which method comprises the steps of:

(a) contacting the test sample with a panel of three or more tumour marker antigens of which three of the tumour marker antigens are p53, SSX1, and either p62 or KOC;
(b) determining the presence or absence of complexes of the tumour marker antigens bound to autoantibodies present in the test sample;
(c) detecting the amount of specific binding between the tumour marker antigens and autoantibodies present in the test sample; and
(d) comparing the amount of specific binding between the tumour marker antigens and the autoantibodies with a previously established relationship between the amount of binding and the likely outcome of treatment,
wherein a change in the amount of specific binding, when compared to controls, predicts that the patient will or will not respond to the lung cancer treatment.

In certain preferred embodiments of the method, the three tumour marker antigens are p53, SSX1, and p62. In certain preferred alternative embodiments of the method, the three tumour marker antigens are p53, SSX1, and KOC.

Herein, the control may be a sample of bodily fluid derived from a subject known to have lung cancer and known not to respond to the lung cancer treatment being tested i.e. to be a non-responding control.

In certain embodiments, the panel of three or more tumour marker antigens comprises p53, SSX1, and p62 and/or KOC, and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and HuD, and wherein the presence of at least complexes containing p53, SSX1, p62 or KOC, and HuD is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and MAGE A4, and wherein the presence of at least complexes containing p53, SSX1, p62 or KOC, and MAGE A4 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and SOX2, and wherein the presence of at least complexes containing p53, SSX1, p62 or KOC, and SOX2 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and CAGE, and wherein the presence of at least complexes containing p53, SSX1, p62 or KOC, and CAGE is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and NY-ESO-1, and wherein the presence of at least complexes containing p53, SSX1, p62 or KOC, and NY-ESO-1 is detected.

In a particularly preferred embodiment, the method involves detecting five or more autoantibodies and the method comprises the step of (a) contacting the test sample with a panel of five or more tumour marker antigens of which five of the tumour marker antigens are p53, SSX1, p62 or KOC, HuD and MAGE A4, and wherein the presence of at least complexes containing p53, p62, SSX1, HuD and MAGE A4 is detected.

In certain embodiments, the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, SSX1, p62, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, NY-ESO-1, p16, p53-95, and p53-C and the presence of complexes containing at least p53, SSX1, p62 or KOC, CAGE, NY-ESO-1, p16, p53-95, and p53-C is detected.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, NY-ESO-1, SOX2, α-enolase-1, and p53-C and the presence of complexes containing at least p53, SSX1, p62 or KOC, NY-ESO-1, SOX2, α-enolase-1, and p53-C is detected.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, HuD, MAGE A4, SOX-2 and CAGE and the presence of complexes containing at least p53, SSX1, p62 or KOC, HuD, MAGE A4, SOX-2 and CAGE is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, HuD, NY-ESO-1, GBU4-5, and p53-C and the presence of complexes containing at least p53, SSX1, p62 or KOC, CAGE, HuD, NY-ESO-1, GBU4-5, and p53-C is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, NY-ESO-1, SOX2, ALDH1, p16, and p53-95 and the presence of complexes containing at least p53, SSX1, p62 or KOC, NY-ESO-1, SOX2, ALDH1, p16, and p53-95 is detected.

In certain embodiments, eight or more autoantibodies are detected, the method comprises the step of (a) contacting the test sample with a panel of eight or more tumour marker antigens of which eight of the tumour marker antigens are p53, SSX1, p62, KOC, CAGE, SOX2, α-enolase-1, and p53-C and the presence of complexes containing at least p53, SSX1, p62, KOC, CAGE, SOX2, α-enolase-1, and p53-C is detected.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, HuD, p16, GBU4-5, and p53-95 and the presence of complexes containing at least p53, SSX1, KOC or p62, CAGE, HuD, p16, GBU4-5, and p53-95 is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CK20, and the presence of complexes containing at least p53, SSX1, p62 or KOC, HuD, MAGE A4, SOX2, NY-ESO-1 and CK20 is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CAGE and the presence of complexes containing at least p53, SSX1, p62 or KOC, HuD, MAGE A4, SOX2, NY-ESO-1 and CAGE is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, CAGE and CK20, and the presence of complexes containing at least p53, SSX1, p62 or KOC, HuD, MAGE A4, SOX2, CAGE and CK20 is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, NY-ESO-1, CAGE and CK20, and the presence of complexes containing at least p53, SSX1, p62 or KOC, HuD, MAGE A4, NY-ESO-1, CAGE and CK20 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, ALDH1, and p53-95 and the presence of complexes containing at least p53, SSX1, p62 or KOC, CAGE, HuD, NY-ESO-1, SOX2, ALDH1, and p53-95 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, p16, and p53-C and the presence of complexes containing at least p53, SSX1, p62 or KOC, CAGE, HuD, NY-ESO-1, SOX2, p16, and p53-C is detected.

In certain embodiments, nine or more autoantibodies are detected, the method comprises the step of (a) contacting the test sample with a panel of nine or more tumour marker antigens of which nine of the tumour marker antigens are p53, SSX1, p62, KOC, HuD, NY-ESO-1, SOX2, α-enolase-1, and p53-C and the presence of complexes containing at least p53, SSX1, p62, KOC, HuD, NY-ESO-1, SOX2, α-enolase-1, and p53-C is detected.

In certain embodiments, nine or more autoantibodies are detected, the method comprises the step of (a) contacting the test sample with a panel of nine or more tumour marker antigens of which nine of the tumour marker antigens are p53, SSX1, KOC or p62, HuD, NY-ESO-1, SOX2, p16, GBU4-5, and p53-95 and the presence of complexes containing at least p53, SSX1, KOC or p62, HuD, NY-ESO-1, SOX2, p16, GBU4-5, and p53-95 is detected.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, SOX2, ALDH1, GBU4-5, and Lmyc2, and the presence of complexes containing at least p53, SSX1, KOC or p62, CAGE, SOX2, ALDH1, GBU4-5, and Lmyc2 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, NY-ESO-1, CAGE and CK20, and the presence of complexes containing at least p53, SSX1, p62 or KOC, HuD, MAGE A4, SOX2, NY-ESO-1, CAGE and CK20 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, CK20, CK8 and KRAS, and the presence of complexes containing at least p53, SSX1, p62 or KOC, HuD, MAGE A4, SOX2, CK20, CK8 and KRAS is detected.

In certain embodiments, nine or more autoantibodies are detected, the method comprises the step of (a) contacting the test sample with a panel of nine or more tumour marker antigens of which nine of the tumour marker antigens are p53, SSX1, p62, KOC, CAGE, HuD, NY-ESO-1, p16, and GBU4-5, and the presence of complexes containing at least p53, SSX1, p62, KOC, CAGE, HuD, NY-ESO-1, p16, and GBU4-5 is detected.

In certain embodiments, nine or more autoantibodies are detected, the method comprises the step of (a) contacting the test sample with a panel of nine or more tumour marker antigens of which nine of the tumour marker antigens are p53, SSX1, KOC or p62, CAGE, HuD, NY-ESO-1, SOX2, ALDH1, and p16, and the presence of complexes containing at least p53, SSX1, KOC or p62, CAGE, HuD, NY-ESO-1, SOX2, ALDH1, and p16 is detected.

In certain embodiments, ten or more autoantibodies are detected, the method comprises the step of (a) contacting the test sample with a panel of ten or more tumour marker antigens of which ten of the tumour marker antigens are p53, SSX1, KOC or p62, CAGE, HuD, SOX2, GBU4-5, α-enolase-1, Lmyc2, and p53-C and the presence of complexes containing at least p53, SSX1, KOC or p62, CAGE, HuD, SOX2, GBU4-5, α-enolase-1, Lmyc2, and p53-C is detected.

In certain embodiments, ten or more autoantibodies are detected, the method comprises the step of (a) contacting the test sample with a panel of ten or more tumour marker antigens of which ten of the tumour marker antigens are p53, SSX1, p62 or KOC, CAGE, HuD, NY-ESO-1, SOX2, ALDH1, p16, and p53-C and the presence of complexes containing at least p53, SSX1, p62 or KOC, CAGE, HuD, NY-ESO-1, SOX2, ALDH1, p16, and p53-C is detected.

In certain embodiments, ten or more autoantibodies are detected, the method comprises the step of (a) contacting the test sample with a panel of ten or more tumour marker antigens of which ten of the tumour marker antigens are p53, SSX1, p62, KOC, CAGE, HuD, NY-ESO-1, SOX2, p16, and α-enolase-1, and the presence of complexes containing at least p53, SSX1, p62, KOC, CAGE, HuD, NY-ESO-1, SOX2, p16, and α-enolase-1 is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C and the presence of complexes containing at least p53, SSX1, p62 or KOC, CAGE, HuD, NY-ESO-1, ALDH1, p16, α-enolase-1, Lmyc2, and p53-C is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS, and the presence of complexes containing at least p53, SSX1, p62 or KOC, HuD, MAGE A4, SOX2, NY-ESO-1, CK20, CK8, p53-95 and KRAS is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS, and the presence of complexes containing at least p53, SSX1, p62 or KOC, HuD, MAGE A4, SOX2, NY-ESO-1, CAGE, CK20, CK8 and KRAS is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS, and the presence of complexes containing at least p53, SSX1, p62 or KOC, HuD, MAGE A4, SOX2, NY-ESO-1, CAGE, GBU4-5, CK8 and KRAS is detected.

It should be noted that the invention is in no way limited to any specific lung cancer treatment. In certain embodiments the lung cancer treatment may be selected from the group consisting of surgery, video-assisted thoracoscopic surgery, radiotherapy, chemotherapy, immunotherapy, radiofrequency ablation, biological therapy, cryotherapy and photodynamic therapy.

Within this embodiment of the invention all limitations discussed above in relation to the various methods of the invention are contemplated in relation to the method of predicting response to a lung cancer treatment.

### Determining an Antibody Profile

The aspects of the invention described above will usually be performed once. However in vitro immunoassays are non-invasive and can be repeated as often as is thought necessary to build up a profile of autoantibody production in a subject, either prior to the onset of lung cancer, as in the screening of “at risk” individuals, or throughout the course of the disease. The methods therefore may be used in determining an antibody profile in a subject having or suspected of having lung cancer.

In certain embodiments, there is provided an in vitro method of determining an autoantibody profile of an individual suffering from lung cancer by detecting three or more autoantibodies in a test sample comprising a bodily fluid from the mammalian subject, wherein three of the autoantibodies are immunologically specific for the tumour marker antigens p53, SSX1, and either p62 or KOC, which method comprises the steps of:

a) contacting the test sample with a panel of three or more tumour marker antigens of which three of the tumour marker antigens are p53, SSX1, and either p62 or KOC; and
b) determining the presence or absence of complexes of the tumour marker antigens bound to autoantibodies present in the test sample, wherein the method is repeated to build up a profile of autoantibody production.

In certain preferred embodiments of the in vitro method, the three tumour marker antigens are p53, SSX1, and p62. In certain preferred alternative embodiments of the in vitro method, the three tumour marker antigens are p53, SSX1, and KOC.

In certain embodiments, the panel of three or more tumour marker antigens comprises p53, SSX1, and p62 and/or KOC, and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and HuD, and wherein the presence of at least complexes containing p53, SSX1, p62 or KOC, and HuD is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and MAGE A4, and wherein the presence of at least complexes containing p53, SSX1, p62 or KOC, and MAGE A4 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and SOX2, and wherein the presence of at least complexes containing p53, SSX1, p62 or KOC, and MAGE A4 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and SOX2, and wherein the presence of at least complexes containing p53, SSX1, p62 or KOC, and SOX2 is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and CAGE, and wherein the presence of at least complexes containing p53, SSX1, p62 or KOC, and CAGE is detected.

In a particularly preferred embodiment, the method involves detecting four or more autoantibodies and the method comprises the step of (a) contacting the test sample with a panel of four or more tumour marker antigens of which four of the tumour marker antigens are p53, SSX1, p62 or KOC, and NY-ESO-1, and wherein the presence of at least complexes containing p53, SSX1, p62 or KOC, and NY-ESO-1 is detected.

In a particularly preferred embodiment, the method involves detecting five or more autoantibodies and the method comprises the step of (a) contacting the test sample with a panel of five or more tumour marker antigens of which five of the tumour marker antigens are p53, SSX1, p62 or KOC, HuD and MAGE A4, and wherein the presence of at least complexes containing p53, SSX1, p62 or KOC, HuD and MAGE A4 is detected.

In certain embodiments, the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, SSX1, p62, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, NY-ESO-1, p16, p53-95, and p53-C and the presence of complexes containing at least p53, SSX1, p62 or KOC, CAGE, NY-ESO-1, p16, p53-95, and p53-C is detected.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, NY-ESO-1, SOX2, α-enolase-1, and p53-C and the presence of complexes containing at least p53, SSX1, p62 or KOC, NY-ESO-1, SOX2, α-enolase-1, and p53-C is detected.

In certain embodiments, seven or more autoantibodies are detected, the method comprises the step of (a) contacting the test sample with a panel of seven or more tumour marker antigens of which seven of the tumour marker antigens are p53, SSX1, p62 or KOC, HuD, MAGE A4, SOX-2 and CAGE and the presence of complexes containing at least p53, SSX1, p62 or KOC, HuD, MAGE A4, SOX-2 and CAGE is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, CAGE, HuD, NY-ESO-1, GBU4-5, and p53-C and the presence of complexes containing at least p53, SSX1, p62 or KOC, CAGE, HuD, NY-ESO-1, GBU4-5, and p53-C is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, NY-ESO-1, SOX2, ALDH1, p16, and p53-95 and the presence of complexes containing at least p53, SSX1, p62 or KOC, NY-ESO-1, SOX2, ALDH1, p16, and p53-95 is detected.

In certain embodiments, eight or more autoantibodies are detected, the method comprises the step of (a) contacting the test sample with a panel of eight or more tumour marker antigens of which eight of the tumour marker antigens are p53, SSX1, p62, KOC, CAGE, SOX2, α-enolase-1, and p53-C and the presence of complexes containing at least p53, SSX1, p62, KOC, CAGE, SOX2, α-enolase-1, and p53-C is detected.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, HuD, p16, GBU4-5, and p53-95 and the presence of complexes containing at least p53, SSX1, KOC or p62, CAGE, HuD, p16, GBU4-5, and p53-95 is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CK20, and the presence of complexes containing at least p53, SSX1, p62 or KOC, HuD, MAGE A4, SOX2, NY-ESO-1 and CK20 is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, NY-ESO-1 and CAGE and the presence of complexes containing at least p53, SSX1, p62 or KOC, HuD, MAGE A4, SOX2, NY-ESO-1 and CAGE is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, SOX2, CAGE and CK20, and the presence of complexes containing at least p53, SSX1, p62 or KOC, HuD, MAGE A4, SOX2, CAGE and CK20 is detected.

In certain embodiments, eight or more autoantibodies are detected, the method comprises the step of (a) contacting the test sample with a panel of eight or more tumour marker antigens of which eight of the tumour marker antigens are p53, SSX1, p62 or KOC, HuD, MAGE A4, NY-ESO-1, CAGE and CK20, and the presence of complexes containing at least p53, SSX1, p62 or KOC, HuD, MAGE A4, NY-ESO-1, CAGE and CK20 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, ALDH1, and p53-95 and the presence of complexes containing at least p53, SSX1, p62 or KOC, CAGE, HuD, NY-ESO-1, SOX2, ALDH1, and p53-95 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, CAGE, HuD, NY-ESO-1, SOX2, p16, and p53-C and the presence of complexes containing at least p53, SSX1, p62 or KOC, CAGE, HuD, NY-ESO-1, SOX2, p16, and p53-C is detected.

In certain embodiments, nine or more autoantibodies are detected, the method comprises the step of (a) contacting the test sample with a panel of nine or more tumour marker antigens of which nine of the tumour marker antigens are p53, SSX1, p62, KOC, HuD, NY-ESO-1, SOX2, α-enolase-1, and p53-C and the presence of complexes containing at least p53, SSX1, p62, KOC, HuD, NY-ESO-1, SOX2, α-enolase-1, and p53-C is detected.

In certain embodiments, nine or more autoantibodies are detected, the method comprises the step of (a) contacting the test sample with a panel of nine or more tumour marker antigens of which nine of the tumour marker antigens are p53, SSX1, KOC or p62, HuD, NY-ESO-1, SOX2, p16, GBU4-5, and p53-95 and the presence of complexes containing at least p53, SSX1, KOC or p62, HuD, NY-ESO-1, SOX2, p16, GBU4-5, and p53-95 is detected.

In certain embodiments, eight or more autoantibodies are detected, the method comprises the step of (a) contacting the test sample with a panel of eight or more tumour marker antigens of which eight of the tumour marker antigens are p53, SSX1, KOC or p62, CAGE, SOX2, ALDH1, GBU4-5, and Lmyc2, and the presence of complexes containing at least p53, SSX1, KOC or p62, CAGE, SOX2, ALDH1, GBU4-5, and Lmyc2 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, NY-ESO-1, CAGE and CK20, and the presence of complexes containing at least p53, SSX1, p62 or KOC, HuD, MAGE A4, SOX2, NY-ESO-1, CAGE and CK20 is detected.

In certain embodiments, nine or more autoantibodies are detected, the method comprises the step of (a) contacting the test sample with a panel of nine or more tumour marker antigens of which nine of the tumour marker antigens are p53, SSX1, p62 or KOC, HuD, MAGE A4, SOX2, CK20, CK8 and KRAS, and the presence of complexes containing at least p53, SSX1, p62 or KOC, HuD, MAGE A4, SOX2, CK20, CK8 and KRAS is detected.

In certain embodiments, nine or more autoantibodies are detected, the method comprises the step of (a) contacting the test sample with a panel of nine or more tumour marker antigens of which nine of the tumour marker antigens are p53, SSX1, p62, KOC, CAGE, HuD, NY-ESO-1, p16, and GBU4-5, and the presence of complexes containing at least p53, SSX1, p62, KOC, CAGE, HuD, NY-ESO-1, p16, and GBU4-5 is detected.

In certain embodiments, nine or more autoantibodies are detected, the method comprises the step of (a) contacting the test sample with a panel of nine or more tumour marker antigens of which nine of the tumour marker antigens are p53, SSX1, KOC or p62, CAGE, HuD, NY-ESO-1, SOX2, ALDH1, and p16, and the presence of complexes containing at least p53, SSX1, KOC or p62, CAGE, HuD, NY-ESO-1, SOX2, ALDH1, and p16 is detected.

In certain embodiments, ten or more autoantibodies are detected, the method comprises the step of (a) contacting the test sample with a panel of ten or more tumour marker antigens of which ten of the tumour marker antigens are p53, SSX1, KOC or p62, CAGE, HuD, SOX2, GBU4-5, α-enolase-1, Lmyc2, and p53-C and the presence of complexes containing at least p53, SSX1, KOC or p62, CAGE, HuD, SOX2, GBU4-5, α-enolase-1, Lmyc2, and p53-C is detected.

In certain embodiments, ten or more autoantibodies are detected, the method comprises the step of (a) contacting the test sample with a panel of ten or more tumour marker antigens of which ten of the tumour marker antigens are p53, SSX1, p62 or KOC, CAGE, HuD, NY-ESO-1, SOX2, ALDH1, p16, and p53-C and the presence of complexes containing at least p53, SSX1, p62 or KOC, CAGE, HuD, NY-ESO-1, SOX2, ALDH1, p16, and p53-C is detected.

In certain embodiments, ten or more autoantibodies are detected, the method comprises the step of (a) contacting the test sample with a panel of ten or more tumour marker antigens of which ten of the tumour marker antigens are p53, SSX1, p62, KOC, CAGE, HuD, NY-ESO-1, SOX2, p16, and α-enolase-1, and the presence of complexes containing at least p53, SSX1, p62, KOC, CAGE, HuD, NY-ESO-1, SOX2, p16, and α-enolase-1 is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C and the presence of complexes containing at least p53, SSX1, p62 or KOC, CAGE, HuD, NY-ESO-1, ALDH1, p16, α-enolase-1, Lmyc2, and p53-C is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS, and the presence of complexes containing at least p53, SSX1, p62 or KOC, HuD, MAGE A4, SOX2, NY-ESO-1, CK20, CK8, p53-95 and KRAS is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS, and the presence of complexes containing at least p53, SSX1, p62 or KOC, HuD, MAGE A4, SOX2, NY-ESO-1, CAGE, CK20, CK8 and KRAS is detected.

In certain embodiments, eleven or more autoantibodies are detected, the method comprises the step of (a) contacting the test sample with a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS, and the presence of complexes containing at least p53, SSX1, p62 or KOC, HuD, MAGE A4, SOX2, NY-ESO-1, CAGE, GBU4-5, CK8 and KRAS is detected.

Within this embodiment of the invention all limitations discussed above in relation to the various methods of the invention are contemplated in relation to the in vitro method of determining an antibody profile.

### Use of a Panel of Tumour Marker Antigens to Detect Lung Cancer

The present invention provides use of a panel of three or more tumour marker antigens for the detection of lung cancer in a mammalian subject by detecting autoantibodies immunologically specific for p53, SSX1, and either p62 or KOC in a test sample comprising a bodily fluid from the mammalian subject.

In certain embodiments, the panel of three or more tumour marker antigens comprises p53, SSX1, and p62 and/or KOC, and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1.

In a particularly preferred embodiment, four or more autoantibodies are detected and the use comprises contacting the test sample with a panel of four or more tumour marker antigens of which four of the tumour marker antigens are p53, SSX1, p62 or KOC, and HuD, and wherein the presence of at least complexes containing p53, SSX1, p62 or KOC, and HuD is detected.

In a particularly preferred embodiment, four or more autoantibodies are detected and the use comprises contacting the test sample with a panel of four or more tumour marker antigens of which four of the tumour marker antigens are p53, SSX1, p62 or KOC, and MAGE A4, and wherein the presence of at least complexes containing p53, SSX1, p62 or KOC, and MAGE A4 is detected.

In a particularly preferred embodiment, four or more autoantibodies are detected and the use comprises contacting the test sample with a panel of four or more tumour marker antigens of which four of the tumour marker antigens are p53, SSX1, p62 or KOC, and SOX2, and wherein the presence of at least complexes containing p53, SSX1, p62 or KOC, and SOX2 is detected.

In a particularly preferred embodiment, four or more autoantibodies are detected and the use comprises contacting the test sample with a panel of four or more tumour marker antigens of which four of the tumour marker antigens are p53, SSX1, p62 or KOC, and CAGE, and wherein the presence of at least complexes containing p53, SSX1, p62 or KOC, and CAGE is detected.

In a particularly preferred embodiment, four or more autoantibodies are detected and the use comprises contacting the test sample with a panel of four or more tumour marker antigens of which four of the tumour marker antigens are p53, SSX1, p62 or KOC, and NY-ESO-1, and wherein the presence of at least complexes containing p53, SSX1, p62 or KOC, and NY-ESO-1 is detected.

In a particularly preferred embodiment, five or more autoantibodies are detected and the method comprises the step of (a) contacting the test sample with a panel of five or more tumour marker antigens of which five of the tumour marker antigens are p53, SSX1, p62 or KOC, HuD and MAGE A4, and wherein the presence of at least complexes containing p53, SSX1, p62 or KOC, HuD and MAGE A4 is detected.

In certain embodiments, the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, p62, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC.

Similarly, for all embodiments where the panel comprises the tumour marker antigen KOC, the invention further encompasses the same panel in which KOC is substituted for p62.

In certain embodiments, eight or more autoantibodies are detected, the use comprises contacting the test sample with a panel of eight or more tumour marker antigens of which eight of the tumour marker antigens are p53, SSX1, p62 or KOC, CAGE, NY-ESO-1, p16, p53-95, and p53-C and the presence of complexes containing at least p53, SSX1, p62 or KOC, CAGE, NY-ESO-1, p16, p53-95, and p53-C is detected.

In certain embodiments, seven or more autoantibodies are detected, the use comprises contacting the test sample with a panel of seven or more tumour marker antigens of which seven of the tumour marker antigens are p53, SSX1, p62 or KOC, NY-ESO-1, SOX2, α-enolase-1, and p53-C and the presence of complexes containing at least p53, SSX1, p62 or KOC, NY-ESO-1, SOX2, α-enolase-1, and p53-C is detected.

In certain embodiments, seven or more autoantibodies are detected, the use comprises contacting the test sample with a panel of seven or more tumour marker antigens of which seven of the tumour marker antigens are p53, SSX1, p62 or KOC, HuD, MAGE A4, SOX-2 and CAGE and the presence of complexes containing at least p53, SSX1, p62 or KOC, HuD, MAGE A4, SOX-2 and CAGE is detected.

In certain embodiments, eight or more autoantibodies are detected, the use comprises contacting the test sample with a panel of eight or more tumour marker antigens of which eight of the tumour marker antigens are p53, SSX1, p62 or KOC, CAGE, HuD, NY-ESO-1, GBU4-5, and p53-C and the presence of complexes containing at least p53, SSX1, p62 or KOC, CAGE, HuD, NY-ESO-1, GBU4-5, and p53-C is detected.

In certain embodiments, eight or more autoantibodies are detected, the use comprises contacting the test sample with a panel of eight or more tumour marker antigens of which eight of the tumour marker antigens are p53, SSX1, p62 or KOC, NY-ESO-1, SOX2, ALDH1, p16, and p53-95 and the presence of complexes containing at least p53, SSX1, p62 or KOC, NY-ESO-1, SOX2, ALDH1, p16, and p53-95 is detected.

In certain embodiments, eight or more autoantibodies are detected, the use comprises contacting the test sample with a panel of eight or more tumour marker antigens of which eight of the tumour marker antigens are p53, SSX1, p62, KOC, CAGE, SOX2, α-enolase-1, and p53-C and the presence of complexes containing at least p53, SSX1, p62, KOC, CAGE, SOX2, α-enolase-1, and p53-C is detected.

In certain embodiments, eight or more autoantibodies are detected, the the use comprises contacting the test sample with a panel of eight or more tumour marker antigens of which eight of the tumour marker antigens are p53, SSX1, KOC or p62, CAGE, HuD, p16, GBU4-5, and p53-95 and the presence of complexes containing at least p53, SSX1, KOC or p62, CAGE, HuD, p16, GBU4-5, and p53-95 is detected.

In certain embodiments, eight or more autoantibodies are detected, the use comprises contacting the test sample with a panel of eight or more tumour marker antigens of which eight of the tumour marker antigens are p53, SSX1, p62 or KOC, HuD, MAGE A4, SOX2, NY-ESO-1 and CK20, and the presence of complexes containing at least p53, SSX1, p62 or KOC, HuD, MAGE A4, SOX2, NY-ESO-1 and CK20 is detected.

In certain embodiments, eight or more autoantibodies are detected, the use comprises contacting the test sample with a panel of eight or more tumour marker antigens of which eight of the tumour marker antigens are p53, SSX1, p62 or KOC, HuD, MAGE A4, SOX2, NY-ESO-1 and CAGE and the presence of complexes containing at least p53, SSX1, p62 or KOC, HuD, MAGE A4, SOX2, NY-ESO-1 and CAGE is detected.

In certain embodiments, eight or more autoantibodies are detected, the use comprises contacting the test sample with a panel of eight or more tumour marker antigens of which eight of the tumour marker antigens are p53, SSX1, p62 or KOC, HuD, MAGE A4, SOX2, CAGE and CK20, and the presence of complexes containing at least p53, SSX1, p62 or KOC, HuD, MAGE A4, SOX2, CAGE and CK20 is detected.

In certain embodiments, eight or more autoantibodies are detected, the use comprises contacting the test sample with a panel of eight or more tumour marker antigens of which eight of the tumour marker antigens are p53, SSX1, p62 or KOC, HuD, MAGE A4, NY-ESO-1, CAGE and CK20, and the presence of complexes containing at least p53, SSX1, p62 or KOC, HuD, MAGE A4, NY-ESO-1, CAGE and CK20 is detected.

In certain embodiments, nine or more autoantibodies are detected, the use comprises contacting the test sample with a panel of nine or more tumour marker antigens of which nine of the tumour marker antigens are p53, SSX1, p62 or KOC, CAGE, HuD, NY-ESO-1, SOX2, ALDH1, and p53-95 and the presence of complexes containing at least p53, SSX1, p62 or KOC, CAGE, HuD, NY-ESO-1, SOX2, ALDH1, and p53-95 is detected.

In certain embodiments, nine or more autoantibodies are detected, the use comprises contacting the test sample with a panel of nine or more tumour marker antigens of which nine of the tumour marker antigens are p53, SSX1, p62 or KOC, CAGE, HuD, NY-ESO-1, SOX2, p16, and p53-C and the presence of complexes containing at least p53, SSX1, p62 or KOC, CAGE, HuD, NY-ESO-1, SOX2, p16, and p53-C is detected.

In certain embodiments, nine or more autoantibodies are detected, the use comprises contacting the test sample with a panel of nine or more tumour marker antigens of which nine of the tumour marker antigens are p53, SSX1, p62, KOC, HuD, NY-ESO-1, SOX2, α-enolase-1, and p53-C and the presence of complexes containing at least p53, SSX1, p62, KOC, HuD, NY-ESO-1, SOX2, α-enolase-1, and p53-C is detected.

In certain embodiments, nine or more autoantibodies are detected, the use comprises contacting the test sample with a panel of nine or more tumour marker antigens of which nine of the tumour marker antigens are p53, SSX1, KOC or p62, HuD, NY-ESO-1, SOX2, p16, GBU4-5, and p53-95 and the presence of complexes containing at least p53, SSX1, KOC or p62, HuD, NY-ESO-1, SOX2, p16, GBU4-5, and p53-95 is detected.

In certain embodiments, eight or more autoantibodies are detected, the use comprises contacting the test sample with a panel of eight or more tumour marker antigens of which eight of the tumour marker antigens are p53, SSX1, KOC or p62, CAGE, SOX2, ALDH1, GBU4-5, and Lmyc2, and the presence of complexes containing at least p53, SSX1, KOC or p62, CAGE, SOX2, ALDH1, GBU4-5, and Lmyc2 is detected.

In certain embodiments, nine or more autoantibodies are detected, the use comprises contacting the test sample with a panel of nine or more tumour marker antigens of which nine of the tumour marker antigens are p53, SSX1, p62 or KOC, HuD, MAGE A4, SOX2, NY-ESO-1, CAGE and CK20, and the presence of complexes containing at least p53, SSX1, p62 or KOC, HuD, MAGE A4, SOX2, NY-ESO-1, CAGE and CK20 is detected.

In certain embodiments, nine or more autoantibodies are detected, the use comprises contacting the test sample with a panel of nine or more tumour marker antigens of which nine of the tumour marker antigens are p53, SSX1, p62 or KOC, HuD, MAGE A4, SOX2, CK20, CK8 and KRAS, and the presence of complexes containing at least p53, SSX1, p62 or KOC, HuD, MAGE A4, SOX2, CK20, CK8 and KRAS is detected.

In certain embodiments, nine or more autoantibodies are detected, the use comprises contacting the test sample with a panel of nine or more tumour marker antigens of which nine of the tumour marker antigens are p53, SSX1, p62, KOC, CAGE, HuD, NY-ESO-1, p16, and GBU4-5, and the presence of complexes containing at least p53, SSX1, p62, KOC, CAGE, HuD, NY-ESO-1, p16, and GBU4-5 is detected.

In certain embodiments, nine or more autoantibodies are detected, the use comprises contacting the test sample with a panel of nine or more tumour marker antigens of which nine of the tumour marker antigens are p53, SSX1, KOC or p62, CAGE, HuD, NY-ESO-1, SOX2, ALDH1, and p16, and the presence of complexes containing at least p53, SSX1, KOC or p62, CAGE, HuD, NY-ESO-1, SOX2, ALDH1, and p16 is detected.

In certain embodiments, ten or more autoantibodies are detected, the the use comprises contacting the test sample with a panel of ten or more tumour marker antigens of which ten of the tumour marker antigens are p53, SSX1, KOC or p62, CAGE, HuD, SOX2, GBU4-5, α-enolase-1, Lmyc2, and p53-C and the presence of complexes containing at least p53, SSX1, KOC or p62, CAGE, HuD, SOX2, GBU4-5, α-enolase-1, Lmyc2, and p53-C is detected.

In certain embodiments, ten or more autoantibodies are detected, the use comprises contacting the test sample with a panel of ten or more tumour marker antigens of which ten of the tumour marker antigens are p53, SSX1, p62 or KOC, CAGE, HuD, NY-ESO-1, SOX2, ALDH1, p16, and p53-C and the presence of complexes containing at least p53, SSX1, p62 or KOC, CAGE, HuD, NY-ESO-1, SOX2, ALDH1, p16, and p53-C is detected.

In certain embodiments, ten or more autoantibodies are detected, the use comprises contacting the test test sample with a panel of ten or more tumour marker antigens of which ten of the tumour marker antigens are p53, SSX1, p62, KOC, CAGE, HuD, NY-ESO-1, SOX2, p16, and α-enolase-1, and the presence of complexes containing at least p53, SSX1, p62, KOC, CAGE, HuD, NY-ESO-1, SOX2, p16, and α-enolase-1 is detected.

In certain embodiments, eleven or more autoantibodies are detected, the the use comprises contacting the test sample with a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C and the presence of complexes containing at least p53, SSX1, p62 or KOC, CAGE, HuD, NY-ESO-1, ALDH1, p16, α-enolase-1, Lmyc2, and p53-C is detected.

In certain embodiments, eleven or more autoantibodies are detected, the the use comprises contacting the test sample with a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS, and the presence of complexes containing at least p53, SSX1, p62 or KOC, HuD, MAGE A4, SOX2, NY-ESO-1, CK20, CK8, p53-95 and KRAS is detected.

In certain embodiments, eleven or more autoantibodies are detected, the use comprises contacting the test sample with a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS, and the presence of complexes containing at least p53, SSX1, p62 or KOC, HuD, MAGE A4, SOX2, NY-ESO-1, CAGE, CK20, CK8 and KRAS is detected.

In certain embodiments, eleven or more autoantibodies are detected, the use comprises contacting the test sample with a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS, and the presence of complexes containing at least p53, SSX1, p62 or KOC, HuD, MAGE A4, SOX2, NY-ESO-1, CAGE, GBU4-5, CK8 and KRAS is detected.

Within this embodiment of the invention all limitations discussed above in relation to the various methods of the invention are contemplated in relation to this use.

Also provided is use of a panel of two or more, three or more, four or more, five or more, six or more or seven or more tumour marker antigens for the detection of lung cancer in a mammalian subject, by detecting autoantibodies immunologically specific for two or more, three or more, four or more, five or more, six or more or seven tumour marker antigens selected from the group consisting of p53, SSX1, SOX2, GBU4-5, HuD, p53-95 and CK8, in a test sample comprising a bodily fluid from the mammalian subject.

### Other Applications of the Method

The methods may be used for the identification of individuals at risk of developing lung cancer in a population of asymptomatic individuals.

The assay method may be repeated on a number of occasions to provide continued monitoring for recurrence of disease. The methods may be used in the detection of recurrent disease in a patient previously diagnosed as having lung cancer who has undergone lung cancer treatment to reduce the amount of lung cancer present.

The methods may be used in assessing the prognosis of a patient diagnosed with lung cancer, in monitoring the progress of lung cancer in a patient, or in monitoring the response of a lung cancer patient to a lung cancer treatment (e.g. surgery, video-assisted thoracoscopic surgery, radiotherapy, chemotherapy, immunotherapy, radiofrequency ablation, biological therapy, cryotherapy and photodynamic therapy).

When the immunoassays are used in monitoring the progress of lung cancer in a subject, the presence of an elevated level of autoantibodies, as compared to a “normal control”, is taken as an indication of the presence of cancer in the patient. The “normal control” may be levels of autoantibodies present in control individuals, preferably age-matched, not having any diagnosis of cancer based on clinical, imaging and/or biochemical criteria. Alternatively, the “normal control” may be a “base-line” level established for the particular subject under test. The “base-line” level may be, for example, the level of autoantibodies present when either a first diagnosis of lung cancer or a diagnosis of recurrent lung cancer was made. Any increase above the base-line level would be taken as an indication that the amount of cancer present in the patient has increased, whereas any decrease below the base-line would be taken as an indication that the amount of cancer present in the patient has decreased.

The immunoassay methods may complement existing methods of screening, diagnosis and surveillance. For example, the methods of the invention may be used in combination with existing methods to confirm a diagnosis of lung cancer. In certain embodiments, the methods of the invention are performed in combination with a CT scan, chest x-ray, PET-CT scan, bronchoscopy and biopsy, thoracoscopy, or other any other suitable method of diagnosing lung cancer.

### F. Kits

The present invention also encompasses a kit for the detection of autoantibodies in a test sample comprising a bodily fluid from a mammalian subject comprising:

(a) a panel of three or more tumour marker antigens of which three of the tumour marker antigens are p53, SSX1, and either p62 or KOC; and
(b) a reagent capable of detecting complexes of the tumour marker antigens bound to autoantibodies present in the test sample.

In certain embodiments of the kit, the three tumour marker antigens are p53, SSX1, and p62. In certain alternative embodiments of the kit, the three tumour marker antigens are p53, SSX1, and KOC.

In certain embodiments, the kit further comprises

(c) means for contacting the tumour marker antigens with a test sample comprising a bodily fluid from a mammalian subject.

Examples of means for contacting the tumour marker antigen with a test sample comprising a bodily fluid from a mammalian subject include the immobilisation of the tumour marker antigen on a chip, slide, wells of a microtitre plate, bead, membrane or nanoparticle.

In certain embodiments, the panel of three or more tumour marker antigens comprises p53, SSX2, and p62 and/or KOC, and one or more tumour marker antigens selected from the group consisting of HuD, MAGE A4, SOX2, NY-ESO-1, CAGE, CK20, GBU4-5, p53-95, p53-C, CK8, KRAS, ALDH1, p16, Lmyc2, and α-enolase-1. Within this embodiment the panel may comprise three, four, five, six, seven, eight, nine, ten, eleven, twelve, thirteen, fourteen, fifteen, sixteen, seventeen, eighteen, or nineteen of the recited tumour marker antigens.

In a particularly preferred embodiment, the kit comprises a panel of four or more tumour marker antigens of which four of the tumour marker antigens are p53, SSX1, p62 or KOC, and HuD.

In a particularly preferred embodiment, the kit comprises a panel of four or more tumour marker antigens of which four of the tumour marker antigens are p53, SSX1, p62 or KOC, and MAGE A4.

In a particularly preferred embodiment, the kit comprises a panel of four or more tumour marker antigens of which four of the tumour marker antigens are p53, SSX1, p62 or KOC, and SOX2.

In a particularly preferred embodiment, the kit comprises a panel of four or more tumour marker antigens of which four of the tumour marker antigens are p53, SSX1, p62 or KOC, and CAGE.

In a particularly preferred embodiment, the kit comprises a panel of four or more tumour marker antigens of which four of the tumour marker antigens are p53, SSX1, p62 or KOC, and NY-ESO-1.

In a particularly preferred embodiment, the kit comprises a panel of five or more tumour marker antigens of which five of the tumour marker antigens are p53, SSX1, p62 or KOC, HuD and MAGE A4.

In certain embodiments, the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, p53-C, CK8, KRAS, ALDH1, p16, Lmyc2, and α-enolase-1.

In certain embodiments, the panel of tumour marker antigens comprises or consists of one of the groups of tumour marker antigens selected from:

(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS;
(viii) p53, SSX1, p62, HuD, MAGE A4, SOX2, NY-ESO-1, CK20, CK8, p53-95, KRAS;
(ix) p53, SSX1, p62, HuD, MAGE A4, SOX2, NY-ESO-1, CAGE, CK20, CK8, KRAS;
(x) p53, SSX1, p62, HuD, MAGE A4, SOX2, NY-ESO-1, CAGE, GBU4-5, CK8, KRAS;
(xi) p53, SSX1, p62, CAGE, HuD, NY-ESO-1, SOX2, ALDH1, p16, p53-C;
(xii) p53, SSX1, p62, CAGE, HuD, NY-ESO-1, GBU4-5, p53-C;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1, p53-95;
(xvi) p53, SSX1, p62, CAGE, HuD, NY-ESO-1, ALDH1, p16, α-enolase-1, Lmyc2, p53-C;
(xvii) p53, SSX1, p62, CAGE, HuD, NY-ESO-1, SOX2, p16, p53-C;
(xviii) p53, SSX1, p62, CAGE, NY-ESO-1, p16, p53-95, p53-C;
(xix) p53, SSX1, p62, NY-ESO-1, SOX2, α-enolase-1, p53-C;
(xx) p53, SSX1, p62, KOC, HuD, NY-ESO-1, SOX2, α-enolase-1, p53-C;
(xxi) p53, SSX1, p62, NY-ESO-1, SOX2, ALDH1, p16, p53-95;
(xxii) p53, SSX1, p62, KOC, CAGE, SOX2, α-enolase-1, p53-C;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5, p53-95;
(xxv) p53, SSX1, KOC, CAGE, HuD, SOX2, GBU4-5, α-enolase-1, Lmyc2, p53-C;
(xxvi) p53, SSX1, KOC, CAGE, HuD, p16, GBU4-5, p53-95; and
(xxvii) p53, SSX1, KOC, CAGE, SOX2, ALDH1, GBU4-5, Lmyc2.

For all embodiments where the panel comprises the tumour marker antigen p62, the invention further encompasses the same panel in which p62 is substituted for KOC. Similarly, for all embodiments where the panel comprises the tumour marker antigen KOC, the invention further encompasses the same panel in which KOC is substituted for p62.

In certain embodiments, the kit comprises a panel of eight or more tumour marker antigens of which eight of the tumour marker antigens are p53, SSX1, p62 or KOC, CAGE, NY-ESO-1, p16, p53-95, and p53-C.

In certain embodiments, the kit comprises a panel of seven or more tumour marker antigens of which seven of the tumour marker antigens are p53, SSX1, p62 or KOC, NY-ESO-1, SOX2, α-enolase-1, and p53-C.

In certain embodiments, the kit comprises a panel of seven or more tumour marker antigens of which seven of the tumour marker antigens are p53, SSX1, p62 or KOC, HuD, MAGE A4, SOX-2 and CAGE.

In certain embodiments, the kit comprises a panel of eight or more tumour marker antigens of which eight of the tumour marker antigens are p53, SSX1, p62 or KOC, CAGE, HuD, NY-ESO-1, GBU4-5, and p53-C.

In certain embodiments, the kit comprises a panel of eight or more tumour marker antigens of which eight of the tumour marker antigens are p53, SSX1, p62 or KOC, NY-ESO-1, SOX2, ALDH1, p16, and p53-95.

In certain embodiments, the kit comprises a panel of eight or more tumour marker antigens of which eight of the tumour marker antigens are p53, SSX1, p62, KOC, CAGE, SOX2, α-enolase-1, and p53-C.

In certain embodiments, the kit comprises a panel of eight or more tumour marker antigens of which eight of the tumour marker antigens are p53, SSX1, KOC or p62, CAGE, HuD, p16, GBU4-5, and p53-95.

In certain embodiments, the kit comprises a panel of eight or more tumour marker antigens of which eight of the tumour marker antigens are p53, SSX1, p62 or KOC, HuD, MAGE A4, SOX2, NY-ESO-1 and CK20.

In certain embodiments, the kit comprises a panel of eight or more tumour marker antigens of which eight of the tumour marker antigens are p53, SSX1, p62 or KOC, HuD, MAGE A4, SOX2, NY-ESO-1 and CAGE.

In certain embodiments, the kit comprises a panel of eight or more tumour marker antigens of which eight of the tumour marker antigens are p53, SSX1, p62 or KOC, HuD, MAGE A4, SOX2, CAGE and CK20.

In certain embodiments, the kit comprises a panel of eight or more tumour marker antigens of which eight of the tumour marker antigens are p53, SSX1, p62 or KOC, HuD, MAGE A4, NY-ESO-1, CAGE and CK20.

In certain embodiments, the kit comprises a panel of nine or more tumour marker antigens of which nine of the tumour marker antigens are p53, SSX1, p62 or KOC, CAGE, HuD, NY-ESO-1, SOX2, ALDH1, and p53-95.

In certain embodiments, the kit comprises a panel of nine or more tumour marker antigens of which nine of the tumour marker antigens are p53, SSX1, p62 or KOC, CAGE, HuD, NY-ESO-1, SOX2, p16, and p53-C.

In certain embodiments, the kit comprises a panel of nine or more tumour marker antigens of which nine of the tumour marker antigens are p53, SSX1, p62, KOC, HuD, NY-ESO-1, SOX2, α-enolase-1, and p53-C.

In certain embodiments, the kit comprises a panel of nine or more tumour marker antigens of which nine of the tumour marker antigens are p53, SSX1, KOC or p62, HuD, NY-ESO-1, SOX2, p16, GBU4-5, and p53-95.

In certain embodiments, the kit comprises a panel of eight or more tumour marker antigens of which eight of the tumour marker antigens are p53, SSX1, KOC or p62, CAGE, SOX2, ALDH1, GBU4-5, and Lmyc2.

In certain embodiments, the kit comprises a panel of nine or more tumour marker antigens of which nine of the tumour marker antigens are p53, SSX1, p62 or KOC, HuD, MAGE A4, SOX2, NY-ESO-1, CAGE and CK20.

In certain embodiments, the kit comprises a panel of nine or more tumour marker antigens of which nine of the tumour marker antigens are p53, SSX1, p62 or KOC, HuD, MAGE A4, SOX2, CK20, CK8 and KRAS.

In certain embodiments, the kit comprises a panel of nine or more tumour marker antigens of which nine of the tumour marker antigens are p53, SSX1, p62, KOC, CAGE, HuD, NY-ESO-1, p16, and GBU4-5.

In certain embodiments, the kit comprises a panel of nine or more tumour marker antigens of which nine of the tumour marker antigens are p53, SSX1, KOC or p62, CAGE, HuD, NY-ESO-1, SOX2, ALDH1, and p16.

In certain embodiments, the kit comprises a panel of ten or more tumour marker antigens of which ten of the tumour marker antigens are p53, SSX1, KOC or p62, CAGE, HuD, SOX2, GBU4-5, α-enolase-1, Lmyc2, and p53-C.

In certain embodiments, the kit comprises a panel of ten or more tumour marker antigens of which ten of the tumour marker antigens are p53, SSX1, p62 or KOC, CAGE, HuD, NY-ESO-1, SOX2, ALDH1, p16, and p53-C.

In certain embodiments, the kit comprises a panel of ten or more tumour marker antigens of which ten of the tumour marker antigens are p53, SSX1, p62, KOC, CAGE, HuD, NY-ESO-1, SOX2, p16, and α-enolase-1.

In certain embodiments, the kit comprises a panel of eleven or more tumour marker antigens of which eleven of the tumour marker antigens are p53, SSX1, p62 or KOC, CAGE, HuD, NY-ESO-1, ALDH1, p16, α-enolase-1, Lmyc2, and p53-C.

In certain embodiments, the kit comprises a panel of eleven or more tumour marker antigens of which eleven of the tumour marker antigens are p53, SSX1, p62 or KOC, HuD, MAGE A4, SOX2, NY-ESO-1, CK20, CK8, p53-95 and KRAS.

In certain embodiments, the kit comprises a panel of eleven or more tumour marker antigens of which eleven of the tumour marker antigens are p53, SSX1, p62 or KOC, HuD, MAGE A4, SOX2, NY-ESO-1, CAGE, CK20, CK8 and KRAS.

In certain embodiments, the kit comprises a panel of eleven or more tumour marker antigens of which eleven of the tumour marker antigens are p53, SSX1, p62 or KOC, HuD, MAGE A4, SOX2, NY-ESO-1, CAGE, GBU4-5, CK8 and KRAS.

Within the kits of the invention, the tumour marker antigen is a naturally occurring protein or polypeptide, a recombinant protein or polypeptide, a synthetic protein or polypeptide, a synthetic peptide, a peptide mimetic, a polysaccharide or a nucleic acid

Within the kits of the invention, the bodily fluid may be selected from the group consisting of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears and wound drainage fluid.

The kits of the invention are suitable for performing any one of the methods of the invention described above. In particular, the kits of the invention are suitable for the detection of lung cancer. Accordingly, in certain embodiments the kits are for the detection of lung cancer.

Also provided herein is a kit for the detection of autoantibodies in a test sample comprising a bodily fluid from a mammalian subject comprising:

(a) a panel of two or more, three or more, four or more, five or more, six or more, seven or more tumour marker antigens of which at least two, at least three, at least four, at least five, at least six or seven of the tumour marker antigens are selected from the group consisting of p53, SSX1, SOX2, GBU4-5, HuD, p53-95 and CK8; and
(b) a reagent capable of detecting complexes of the tumour marker antigens bound to autoantibodies present in the test sample.

Also provided herein is a kit for the detection of autoantibodies in a test sample comprising a bodily fluid from a mammalian subject comprising:

(a) a panel of seven or more tumour marker antigens of which seven of the tumour marker antigens are p53, SSX1, SOX2, GBU4-5, HuD, p53-95 and CK8; and
(b) a reagent capable of detecting complexes of the tumour marker antigens bound to autoantibodies present in the test sample.

The invention will now be further understood with reference to the following non-limiting examples.

### EXAMPLES

### Example 1: Methodology for Measuring Autoantibodies to Tumour-Associated Proteins (Antigens)

Samples of tumour marker antigens may be prepared by recombinant expression, following analogous methods to those described in WO 99/58978 (the contents of which are incorporated herein by reference). Briefly, cDNAs encoding the marker antigens of interest (Table 1) were cloned into the pET21 or pET45 vector (Invitrogen) modified to encode a biotin tag and a 6×histidine tag (His tag) to aid in purification of the expressed protein. The resulting clones were grown in BL21(DE3) E. coli, with the bacteria subsequently lysed. The expressed antigens were recovered via nickel chelate affinity columns (HiTrap, commercially available from GE Healthcare), following the manufacturer's protocol. The purity, specificity and yield of expressed protein were assessed by SDS-PAGE, Western blot and protein assay prior to storage.

A negative control protein, VOL, was produced by transforming BL21(DE3) E. coli with empty pET21 vector (i.e. no cDNA encoding tumour associated antigen). The expressed and purified protein includes the same His and biotin tag sequences found on the recombinant tumour associated antigens and allows correction for non-specific autoantibody binding to residual bacterial contaminants.

TABLE 1
Antigen details and accession numbers

| | | | Protein | AAs of accession sequence |
|---|---|---|---|---|
| Alias | GeneID | Gene name | Accession | (exemplary antigen sequence) |
| α-enolase | 2023 | ENO1 | NP_001419.1 | 1-434 (SEQ ID NO: 1) |
| CAGE | 168400 | DDX53 | AAH67878.1 | 1-631 (SEQ ID NO: 2) |
| CK20 | 54474 | KRT20 | NP_061883.1 | 1-424 (SEQ ID NO: 3) |
| CK8 | 3856 | KRT8 | NP_001243222.1 | 1-483 (SEQ ID NO: 4) |
| EGFR1-ECD | 1956 | EGFR | NP_001333827.1 | 26-627 (SEQ ID NO: 5) |
| EGFR1-EP | 1956 | EGFR | NP_958439.1 | 311-486 (SEQ ID NO: 6) |
| EGFR1-KD | 1956 | EGFR | NP_001333827.1 | 668-1022 (SEQ ID NO: 7) |
| EGFR2 | 1956 | EGFR | BAI46646.1 | 26-403 (SEQ ID NO: 8) |
| EGFR-L858R | 1956 | EGFR | NP_001333827.1 | 668-1021 (SEQ ID NO: 9) |
| EGFR-VIII | 1956 | EGFR | NP_001333870.1 | 25-360 (SEQ ID NO: 10) |
| GBU4-5 | 91646 | TDRD12 | NP_001353031.1 | 1-374 (SEQ ID NO: 11) |
| HuD | 1996 | ELAVL4 | AAH36071.1 | 1-366 (SEQ ID NO: 12) |
| KRAS | 3845 | KRAS | AAH13572.1 | 1-188 (SEQ ID NO: 13) |
| MAGE A4 | 4103 | MAGEA4 | NP_001011548.1 | 1-317 (SEQ ID NO: 14) |
| NY-ESO-1 | 1485 | CTAG1B | NP_640343.1 | 1-180 (SEQ ID NO: 15) |
| p16 | 1029 | CDKN2A | NP_000068.1 | 49-156 (SEQ ID NO: 16) |
| p53 | 7157 | TP53 | NP_000537 | 1-394 (SEQ ID NO: 17) |
| p53-95 | 7157 | TP53 | NP_000537 | 1-95 (SEQ ID NO: 18) |
| p53-C | 7157 | TP53 | NP_000537 | 221-393 (SEQ ID NO: 23) |
| p62 | 10644 | IGF2BP2 | NP_006539.3 | 1-599 (SEQ ID NO: 19) |
| SOX2 | 6657 | SOX2 | NP_003097.1 | 1-317 (SEQ ID NO: 20) |
| SSX1 | 6756 | SSX1 | NP_001265620.1 | 1-188 (SEQ ID NO: 21) |
| VOL | 29491824 | RM25_RS08835 | AAA89090.1 | 1-129 (SEQ ID NO: 22) |
| ALDH1 | 216 | ALDH1A1 | NP_000680.2 | 1-501 (SEQ ID NO: 24) |
| KOC | 10643 | IGF2BP3 | NP_006538.2 | 1-579 (SEQ ID NO: 25) |
| Lmyc2 | 4610 | MYCL | NP_001028254.1 | 41-236 (SEQ ID NO: 26) |

The GeneID and Protein Accession numbers can be found on the NCBI website available at www.ncbi.nlm.nih.gov.

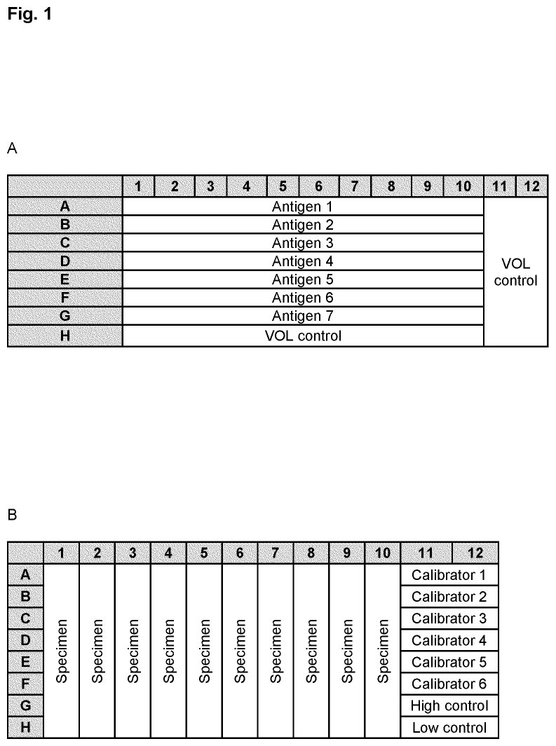

Antigens and VOL (negative control) were diluted to appropriate concentrations (160 and/or nM) in borate coating buffer (pH 8.5) and dispensed at 100 μl/well into the wells of a microtitre plate according to the plate layout (FIG. 1A) using an automated liquid handling system. Plates were covered and stored at +18 to +22° C. for 18 to 24 hours after which time all wells were washed with PBS+0.1% tween 20 using an automated plate washer. Plates were tapped dry on absorbent paper and blocking buffer was added at 200 μl/well.

The plates were then stored at +18 to +22° C. for 2 hours after which time the well contents were aspirated and the plates were allowed to air dry overnight.

Serum samples were defrosted, mixed and diluted 1/110 in Specimen Antibody Diluent (either PBS-1% BSA+0.1% Tween 80+0.01% Pluronic F-127, or PBS+0.1% casein) at +18 to +22° C. Each diluted serum sample was dispensed at 100 μl/well into the microtitre plates according to the plate layout in FIG. 1B.

On-plate calibrator, high control and low control all using Chimeric Human-Rabbit anti-His tag monoclonal antibody (Sigma) were diluted in Specimen Antibody Diluent and dispensed at 100 μl/well into the microtitre plates according to the plate layout in FIG. 1B. Plates were covered and incubated for 1.5 hours at room temperature with shaking.

Plates were washed as above and horseradish peroxidase conjugated rabbit anti-human immunoglobulin diluted in Specimen Antibody Diluent was dispensed at 100 μl/well into all wells of the microtitre plates. Plates were then incubated at room temperature for 1 hour with shaking. Plates were washed as described above.

Pre-prepared 3,3′,5,5′-tetramethylbenzidine (TMB) substrate was added to each plate at 100 μl/well and incubated on the bench for 15 minutes. Plates were gently tapped to mix. Stop solution (1 M HCl) was added after 15 minutes at 100 μl/well. The optical density of each well was determined at 450 nm using a standard spectrophotometric plate reader.

### Example 2: Detection of Autoantibodies in Chinese Lung Cancer Patients Using the Commercially Available EarlyCDT Lung Test Kit

The EarlyCDT Lung kit assay (Oncimmune Limited, Nottingham, UK) was carried out according to the Instructions for Use (IFU) with the cut-offs recommended by the manufacturer applied. Sera samples were collected in China from a population of Chinese ethnicity with the clinical and demographic status of this cohort (Cohort 1) given in Table 2.

TABLE 2
Demographic status of Cohort 1 consisting of lung cancer cases
and control cohorts of individuals with either benign lung
disease or no evidence of malignancy (healthy normals)

| Demographic | Lung cancer | Benign | Normal | |
|---|---|---|---|---|
| Number | 78 | 95 | 77 | 109 |
| Mean age | Unknown | Unknown | Unknown | 30.0 |
| Age range | Unknown | Unknown | Unknown | 18-56 |
| % Male | Unknown | Unknown | Unknown | 52.3% |

Briefly, the EarlyCDT Lung test detects autoantibodies (AAb) to a panel of seven antigens (Table 3). The results (Table 3) show that for this cohort, the EarlyCDT Lung test has a sensitivity for lung cancer of 32.1% and specificity of 79.1% and 76.8% for healthy and benign control cohorts, respectively, using the established cut-offs. It is therefore apparent that both the sensitivity and specificity with this cohort of samples from Chinese patients were lower than the performance claims stated (41% sensitivity and 90% specificity).

These results suggest that the EarlyCDT Lung test panel, developed and validated for early detection of lung cancer in Western patients, may not be optimal for achieving the same purpose in Chinese patients and that alternative cut offs or autoantibodies may need to be measured in order to account for ethnic differences between the two regional populations.

TABLE 3
Positivity of individual autoantibodies (AAb) and the panel in each patient cohort (lung cancer
cases, benign lung disease controls and healthy
normal controls) using the EarlyCDT Lung test kit

| | Lung Cancer | | Benign | | Normal | |
|---|---|---|---|---|---|---|
| | AAb | | AAb | | AAb | |
| Antigens | Positives | Sensitivity | Positives | Specificity | Positives | Specificity |
| p53 | 11/78 | 14.1% | 6/95 | 93.7% | 13/186 | 93.0% |
| SOX2 | 0/78 | 0.0% | 0/95 | 100.0% | 0/186 | 100.0% |
| CAGE | 5/78 | 6.4% | 1/95 | 98.9% | 10/186 | 94.6% |
| NY-ESO-1 | 3/78 | 3.8% | 9/95 | 90.5% | 11/186 | 94.1% |
| GBU4-5 | 3/78 | 3.8% | 5/95 | 94.7% | 9/186 | 95.2% |
| MAGE A4 | 7/78 | 9.0% | 12/95 | 87.4% | 15/186 | 91.9% |
| HuD | 1/78 | 1.3% | 0/95 | 100.0% | 0/186 | 100.0% |
| Panel | 25/78 | 32.1% | 22/95 | 76.8% | 39/186 | 79.0% |

### Example 3: Detection of Autoantibodies in Chinese Lung Cancer Patients Using the Commercially Available CancerProbe Test

An autoantibody test (English name—Seven Kinds of Autoantibodies Test Kit (ELISA), “CancerProbe”) for early detection of lung cancer is marketed in China (manufactured by Hangzhou Cancer Probe Biotechnology Company, Hangzhou, China). This test also measures autoantibodies to a panel of seven antigens, four of which are also present in the EarlyCDT Lung test (p53, SOX2, CAGE and GBU4-5) and three are different (GAGE-7, MAGE A1 and PGP9.5).

Autoantibodies were measured for a set of samples (n=62; subset of Cohort 1, Table 2) collected in China from a population of Chinese ethnicity using the CancerProbe test according to the Instructions for Use (IFU).

The performance of the CancerProbe test (Table 4) was compared to the performance of the EarlyCDT Lung test for the same subset of samples (Table 5). Autoantibody levels were measured according to the IFU for each test.

TABLE 4
Positivity of individual autoantibodies (AAb) and the whole panel in each patient
cohort (lung cancer cases, benign lung disease controls and healthy normal controls)
for the CancerProbe test

| | Lung Cancer | | Benign | | Normal | |
|---|---|---|---|---|---|---|
| | AAb | | AAb | | AAb | |
| Antigen | Positives | Sensitivity | Positives | Specificity | Positives | Specificity |
| p53 | 4/21 | 19.1% | 2/21 | 90.5% | 0/20 | 100.0% |
| SOX2 | 5/21 | 23.8% | 3/21 | 85.7% | 1/20 | 95.0% |
| CAGE | 2/21 | 9.5% | 1/21 | 95.2% | 0/20 | 100.0% |
| GBU4-5 | 0/21 | 0.0% | 2/21 | 95.2% | 0/20 | 100.0% |
| PGP9.5 | 0/21 | 0.0% | 5/21 | 76.2% | 1/20 | 95.00% |
| GAGE-7 | 1/21 | 4.8% | 0/21 | 100.0% | 1/20 | 95.0% |
| MAGE A1 | 2/21 | 9.5% | 0/21 | 100.0% | 1/20 | 95.0% |
| Panel | 9/21 | 42.9% | 8/21 | 61.9% | 4/20 | 80.0% |

TABLE 5
Positivity of individual autoantibodies (AAb) and the whole panel in each patient
cohort (lung cancer cases, benign lung disease controls and healthy normal controls) for
EarlyCDT Lung test

| | Lung Cancer | | Benign | | Normal | |
|---|---|---|---|---|---|---|
| | AAb | | AAb | | AAb | |
| Antigen | Positives | Sensitivity | Positives | Specificity | Positives | Specificity |
| p53 | 5/21 | 23.8% | 2/21 | 90.5% | 0/20 | 100.0% |
| SOX2 | 0/21 | 0.0% | 0/21 | 100.0% | 0/20 | 100.0% |
| CAGE | 3/21 | 14.3% | 1/21 | 95.2% | 1/20 | 95.0% |
| NY-ESO-1 | 3/21 | 14.3% | 5/21 | 76.2% | 2/20 | 90.0% |
| GBU4-5 | 2/21 | 9.5% | 2/21 | 90.5% | 2/20 | 90.00% |
| MAGE A4 | 4/21 | 19.1% | 8/21 | 91.9% | 4/20 | 80.0% |
| HuD | 1/21 | 4.8% | 0/21 | 100.0% | 0/20 | 100.0% |
| Panel | 11/21 | 52.4% | 11/21 | 47.6% | 5/20 | 75.0% |

The results show that for this cohort, the CancerProbe test has a sensitivity of 42.9% and a specificity of 61.9% and 80.0% for benign and healthy control groups, respectively. The results also show that, for the same group of patients, the EarlyCDT Lung test has a sensitivity of 52.4% and a specificity of 47.6% and 75.0% for benign and healthy control groups, respectively.

### Example 4: Determination of Antigen Panels Optimized for Early Detection of Lung Cancer in a Chinese Population

The following data were obtained from a study to explore the sensitivity and specificity of the development assay (methodology detailed in Example 1) for an independent cohort of patients, investigating panel performance for panels of up to 14 markers selected from p53, p62, SSX1, HuD, MAGE A4, SOX2, NY-ESO-1, CAGE, CK20, GBU4-5, p53-95, CK8, KRAS and α-enolase. All antigens were coated at 50 nM. The CancerProbe test was also carried out for the same cohort, following the IFU.

The clinical and demographic status of subjects included in this study (Cohort 2) are given in Table 6. They are a completely independent group of patients to those investigated in Examples 2 and 3 (Cohort 1 and subset of Cohort 1, respectively).

TABLE 6
Demographic status of Cohort 2 consisting of lung cancer cases
and control cohorts of individuals with benign lung disease

| Demographic | Lung cancer | Benign |
|---|---|---|
| Number | 98 | 55 |
| Mean age | 58.6 | 51.7 |
| Age range | 30-82 | 15-77 |
| % Male | 49.0% | 66.7% |
| Gender & age unknown | 0 | 1 |

(i) Panel of Seven Antigens that are in the EarlyCDT Lung Test

Optimal cut-offs, in RU, were determined using a simulated annealing based multivariate cut-off optimization algorithm.

The results (Table 7) show when an optimized set of cut offs for this Chinese cohort are applied to the assay results, this panel, which is equivalent to the EarlyCDT Lung test panel, has a sensitivity for lung cancer of 31.6% and specificity of 90.9% for a benign control cohort. It is apparent that both the sensitivity and specificity are lower than the stated EarlyCDT Lung test performance claims 41% sensitivity and 91% specificity), for this Chinese cohort. This would suggest that the panel developed and validated for early detection of lung cancer in Western patients may not be optimal for achieving the same purpose in Chinese patients and that alternative autoantibodies may need to be measured in order to account for ethnic differences between the two regional populations.

TABLE 7
Positivity of individual autoantibodies (AAb) and the EarlyCDT
Lung test panel in each patient cohort (lung cancer cases
and benign lung disease controls) for the indicated cut offs

| | | Lung Cancer | | Benign | |
|---|---|---|---|---|---|
| | Cut off | AAb | | AAb | |
| Antigen | (RU) | Positives | Sensitivity | Positives | Specificity |
| p53 | 2.72 | 17/98 | 17.4% | 1/55 | 98.2% |
| SOX2 | 4.01 | 6/98 | 6.1% | 2/55 | 96.4% |
| CAGE | 2.92 | 5/98 | 5.1% | 2/55 | 96.4% |
| NY-ESO-1 | 5.43 | 2/98 | 2.0% | 0/55 | 100.0% |
| GBU4-5 | 3.77 | 1/98 | 1.0% | 0/55 | 100.0% |
| MAGE A4 | 4.21 | 2/98 | 2.0% | 0/55 | 100.0% |
| HuD | 2.75 | 5/98 | 5.1% | 1/55 | 98.20% |
| Panel | n/a | 31/98 | 31.6% | 5/55 | 90.9% |

### (ii) CancerProbe Test Panel

The CancerProbe test results and performance for the same cohort of patients are shown in Table 8 and result in an overall sensitivity of 26.5% and specificity of 96.4% for the benign control group.

TABLE 8
Positivity of individual autoantibodies (AAb) and the whole panel
of the Cancerprobe test in each patient cohort (lung cancer cases,
benign lung disease controls and healthy normal controls)

| | Lung Cancer | | Benign | |
|---|---|---|---|---|
| | AAb | | AAb | |
| Antigen | Positives | Sensitivity | Positives | Specificity |
| p53 | 1/98 | 1.0% | 0/55 | 100% |
| SOX2 | 11/98 | 11.2% | 0/55 | 100% |
| CAGE | 4/98 | 4.1% | 2/55 | 96.4% |
| GBU4-5 | 9/98 | 9.2% | 0/55 | 100% |
| PGP9.5 | 2/98 | 2.0% | 0/55 | 100% |
| GAGE-7 | 7/98 | 7.1% | 0/55 | 100% |
| MAGE A1 | 3/98 | 3.1% | 0/55 | 100% |
| Panel | 26/98 | 26.5% | 2/55 | 96.4% |

To determine the sensitivity possible with reduced specificity, a simulated annealing optimisation was performed based on the examined cohort. Cut-off sets discovered by simulated annealing optimisation suggested a maximum sensitivity of 40.8% for a specificity of 90.9%, however this optimisation has a high potential for overfitting due to the small size of the control cohort.

(iii) Alternative Test Panels of 3-14 Markers

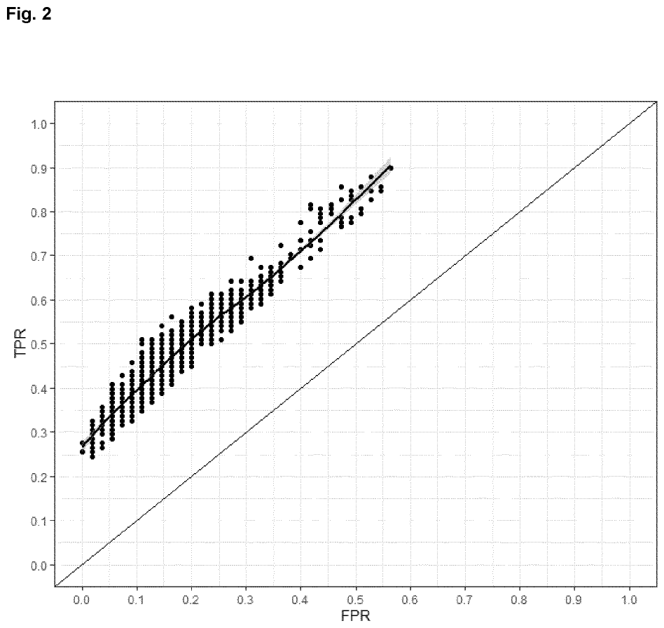

Optimal cut-offs, in RU, for this cohort's assay results for panels of 14, 9, 5 and 3 markers were determined using a simulated annealing based multivariate cut-off optimization algorithm. This approach identified a number of different panels of varying sizes and performance (Tables 9-12, FIGS. 2-5) which can be directly compared to the CancerProbe test performance as they have been determined using the exact same group of patients. For the panels identified below, with a specificity of 90.9%, the panel sensitivities range from 37.8 to 48.0%, and as such, all demonstrate superior performance to the CancerProbe test for the same Chinese cohort.

TABLE 9
Performance characteristics for the top ranking panels of FIG. 2

| | Cut offs (RU) | | | | |
|---|---|---|---|---|---|
| | Cut-off | Cut-off | Cut-off | Cut-off | Cut-off |
| AAb | set 1 | set 2 | set 3 | set 4 | set 5 |
| p53 | 3.358 | 3.358 | 3.358 | 3.358 | 2.761 |
| P62 | 4.438 | 4.438 | 4.438 | 4.438 | 4.438 |
| SSX1 | 2.869 | 4.607 | 4.607 | 2.869 | 2.869 |
| HuD | 2.573 | 2.573 | 2.573 | 2.986 | 2.986 |
| MAGE A4 | 3.808 | 3.507 | 3.808 | 3.507 | 3.507 |
| SOX2 | 4.582 | 5.198 | 5.198 | 5.198 | 5.198 |
| CK8 | 3.521 | 3.143 | 3.521 | 3.521 | n/a |
| GBU4-5 | n/a | n/a | 3.648 | n/a | n/a |
| p53-95 | n/a | 5.521 | n/a | n/a | n/a |
| NY-ESO-1 | 2.382 | 5.382 | 5.382 | n/a | 5.382 |
| CAGE | 3.048 | n/a | 3.048 | n/a | 3.048 |
| α-Enolase | n/a | n/a | n/a | n/a | n/a |
| KRAS | 3.991 | 3.991 | 3.991 | 3.991 | n/a |
| CK20 | 4.504 | 4.504 | n/a | 3.713 | 3.713 |
| Panel Sensitivity | 45.9% | 43.9% | 42.9% | 42.9% | 48.0% |
| Panel Specificity | 90.9% | 90.9% | 90.9% | 90.9% | 90.9% |

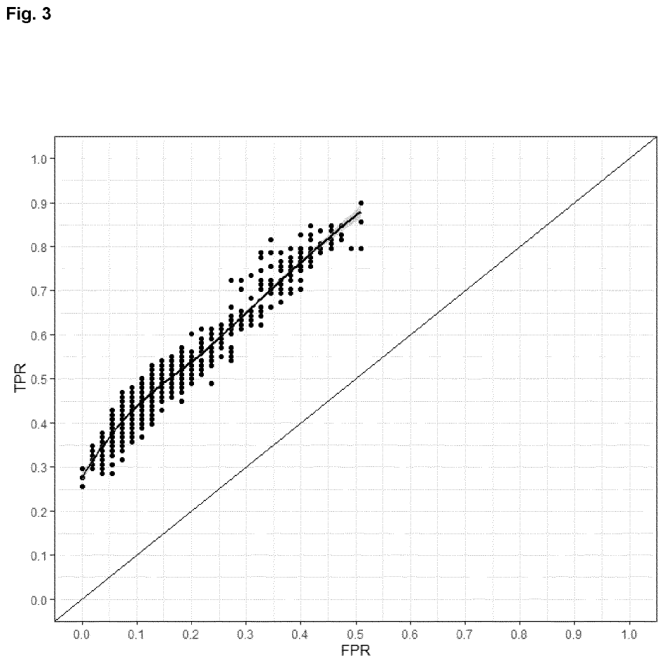

TABLE 10
Performance characteristics for the top ranking panels of FIG. 3.

| | Cut offs (RU) | | | | | |
|---|---|---|---|---|---|---|
| | Cut-off | Cut-off | Cut-off | Cut-off | Cut-off | Cut-off |
| AAb | set 1 | set 2 | set 3 | set 4 | set 5 | set 6 |
| p53 | 2.761 | 2.761 | 2.761 | 2.761 | 2.761 | 2.761 |
| p62 | 4.293 | 4.438 | 4.438 | 4.438 | 4.438 | 4.293 |
| SSX1 | 2.869 | 2.869 | 2.869 | 2.869 | 2.869 | 2.869 |
| HuD | 2.573 | 2.986 | 2.986 | 2.573 | 2.573 | 2.573 |
| MAGE A4 | 3.808 | 3.507 | 3.507 | 3.507 | 3.808 | 3.808 |
| SOX2 | 5.198 | 5.198 | 5.198 | n/a | 5.198 | 5.198 |
| NY-ESO-1 | 5.023 | n/a | 5.382 | 5.382 | n/a | 5.023 |
| CAGE | 3.048 | 3.048 | 3.048 | 3.048 | 3.048 | n/a |
| CK20 | n/a | 3.713 | 3.713 | 4.504 | n/a | 4.504 |
| Panel Sensitivity | 48.0% | 48.0% | 48.0% | 48.0% | 46.9% | 45.9% |
| Panel Specificity | 90.9% | 90.9% | 90.9% | 90.9% | 92.7% | 92.7% |

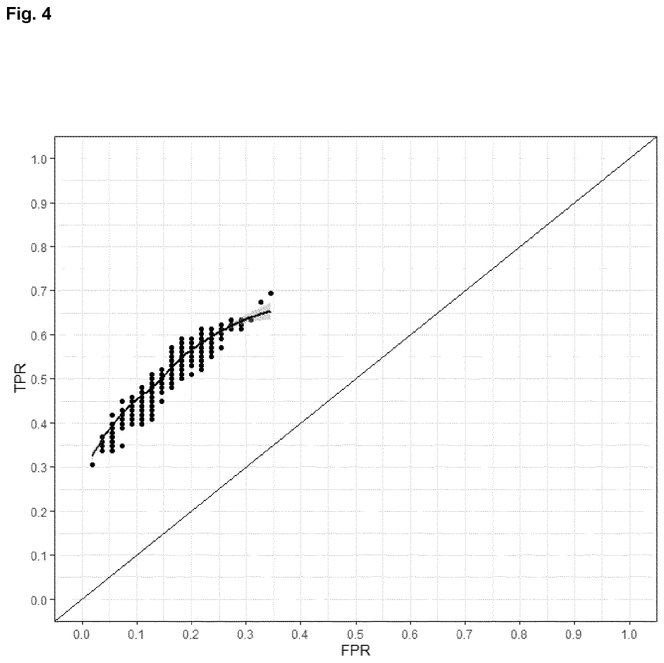

TABLE 11
Performance characteristics for the top ranking panels of FIG. 4

| | Cut offs (RU) | | | | | | |
|---|---|---|---|---|---|---|---|
| | | Cut-off | Cut-off | Cut-off | Cut-off | Cut-off | Cut-off |
| AAb | Cut-off set 1 | set 2 | set 3 | set 4 | set 5 | set 6 | set 7 |
| p53 | 2.761 | 2.692 | 2.761 | 2.761 | 2.761 | 2.761 | 2.761 |
| p62 | 4.293 | 4.438 | 4.438 | 4.293 | 4.438 | 4.438 | 4.438 |
| SSX1 | 2.869 | 2.869 | 2.869 | 2.869 | 2.869 | 2.869 | 2.869 |
| HuD | 2.573 | 2.573 | 2.573 | 2.534 | 2.573 | 2.482 | 2.573 |
| MAGE A4 | 3.327 | 3.507 | 3.507 | 3.507 | 3.507 | 3.507 | n/a |
| Panel Sensitivity | 45.9% | 45.9% | 45.9% | 45.9% | 45.9% | 45.9% | 39.8% |
| Panel Specificity | 90.9% | 90.9% | 90.9% | 90.9% | 90.9% | 90.9% | 94.5% |

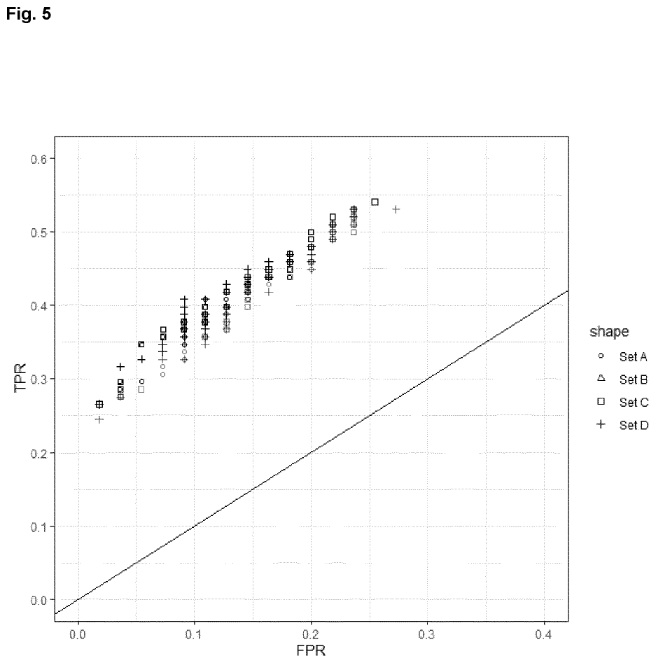

TABLE 12
Performance characteristics for cut off sets A-D of FIG. 5

| Panel | Sensitivity (%) | Specificity (%) |
|---|---|---|
| Set A: p53. HuD, SSX1 | 37.8 | 90.9 |
| Set B: p62, HuD, SSX1 | n/a* | n/a* |
| Set C: p53, p62, HuD | 37.8 | 90.9 |
| Set D: p53, p62, SSX1 | 40.8 | 90.9 |

*In the absence of p53, the optimisation was unable to find panels with performance that satisfied the search constraints

These results suggest that panels of 3-14 markers incorporating at least p53, SSX1 and p62 in each, perform the same or better than the CancerProbe test for the same cohort. Even when results are comparable to the CancerProbe test result (e.g. the three marker panel of p53, p62, SSX1), the simplicity of using only three tumour marker antigens is advantageous.

### Example 5: Detection of Autoantibodies for an Expanded Set of Antigens in Chinese Lung Cancer Patients Using the Development Assay

The following data were obtained from a feasibility study to assess the sensitivity and specificity of a development assay (methodology detailed in Example 1) for a panel of up to markers (Table 1) which includes those markers used in the EarlyCDT Lung kit (Example 2). This was carried out to assess performance of the EarlyCDT Lung panel for a larger independent cohort. This study aimed to determine whether optimisation of marker cut-offs for a Chinese population and/or replacement of some markers improved the test performance.

Antigens were coated at either 50 nM (p53, MAGE A4, SOX2, HuD and NY-ESO-1), 160 nM (CAGE and GBU4-5) or at both concentrations (CK8, CK20, EGFR1-ECD, EGFR1-EP, EGFR1-KD, EGFR2. EGFR-L858R, EGFR-VIII, KRAS, p16, p53-95, p62, α-enolase and SSX1).

The clinical and demographic status of subjects included in the study (Cohort 3) is given in Table 13.

TABLE 13
Demographic status of Cohort 3 consisting of lung cancer cases
and control cohorts of individuals with no history of malignancy
(healthy normals) for use in the development assay

| | | |
|---|---|---|
| | Lung cancer | |
| Demographic factor | patients | Normal |

| | | |
|---|---|---|
| Number | 148 | 145 |
| Mean age | 60.8 | 29.4 |

| | | |
|---|---|---|
| Age range | 35-76 | 18-56 |

| % Male | 51.4% | | 50% |
|---|---|---|---|
| Diagnosis: | | | n/a |
| Adenocarcinoma | 100 | (67.6%) | |
| Squamous | 26 | (17.6%) | |
| Adenosquamous | 3 | (2.0%) | |
| NSCLC | 1 | (0.7%) | |
| SCLC | 15 | (10.1%) | |
| Large cell | 1 | (0.7%) | |
| Sarcamatoid | 1 | (0.7%) | |
| Unknown | 1 | (0.7%) | |
| Stage: | | | n/a |
| I | 8 | (5.4%) | |
| Ia | 64 | 43.2%) | |
| Ib | 24 | (16.2%) | |
| II | 7 | (4.7%) | |
| IIa | 13 | (8.8%) | |
| IIb | 3 | (2.0%) | |
| IIIa | 27 | (18.2%) | |
| Unknown | 2 | (1.4%) | |

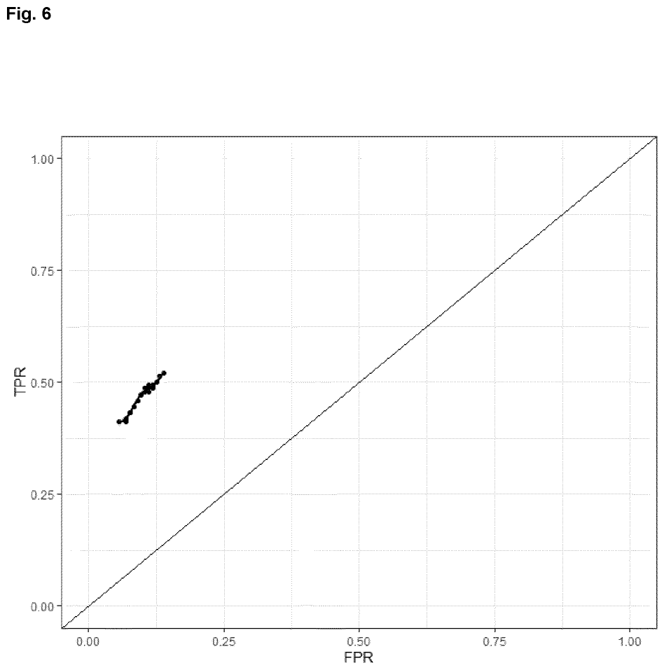

Optimal cut-offs, in reference units (RU), for the cohort's assay results for panels of seven markers were determined using a simulated annealing based multivariate cut-off optimization algorithm. This approach identified a number of different panels and the ROC scatter plot (FIG. 6) shows the range of sensitivity and specificity combinations that could be gained by various cut-off combinations. The best performing seven marker panel is detailed in Table 14 with sensitivity of 41.2% and specificity of 94.4%.

TABLE 14
Positivity of individual autoantibody (AAb) markers and
the whole panel of seven markers in each patient cohort
(lung cancer cases and healthy normal controls) for the
indicated cut offs for the development assay cohort

| | | Lung Cancer | | Normal | |
|---|---|---|---|---|---|
| | Cut off | AAb | | AAb | |
| Antigen | (RU)^{a} | Positives | Sensitivity | Positives | Specificity |
| p53 | 3.925 | 12/148 | 8.1% | 0/144 | 100.0% |
| SOX2 | 1.435 | 35/148 | 23.6% | 5/144 | 96.5% |
| GBU4-5 | 3.893^{b} | 6/148 | 4.1% | 0/144 | 100.0% |
| HuD | 3.819 | 3/148 | 2.0% | 0/144 | 100.0% |
| p53-95 | 4.788 | 2/148 | 1.4% | 0/144 | 100.0% |
| CK8 | 2.770 | 13/148 | 8.8% | 3/144 | 97.9% |
| SSX1 | 3.243 | 5/148 | 3.4% | 0/144 | 100.0% |
| Panel | n/a | 62/148 | 41.2% | 6/144 | 94.4% |

^{a}Cut offs for autoantibodies to antigens coated at 50 nM or
^{b}160 nM.

These analyses show that by exchanging some of the markers and optimizing cut offs for a Chinese population, the performance of a panel of seven markers can be raised to comparable levels to those stated for the EarlyCDT Lung test in a Western population.

### Example 6: Determination of Antigen Panels Optimized for Early Detection of Lung Cancer in a Western Population

The following data were obtained from a study to explore panel performance for panels of up to 19 markers selected from p53, SSX1, p62, KOC, HuD, MAGE A4, SOX2, NY-ESO-1, CAGE, CK20, GBU4-5, p53-95, p53-C, CK8, KRAS, ALDH1, p16, Lmyc2 and α-enolase for three independent cohorts of patients living in Western Europe or the USA. All antigens were coated at 50 nM and 160 nM

The clinical and demographic status of subjects included in this study (training cohort, test cohort and validation cohort) are given in Tables 15-17. They are a completely independent group of patients to those investigated in Examples 2, 3 and 4 which were of Chinese origin.

TABLE 15
Demographic status of Training Cohort consisting of lung cancer
cases and control cohorts of individuals with no lung disease

| | | |
|---|---|---|
| | Lung cancer | Normal |
| Demographic | (n = 219) | (n = 222) |

| | | |
|---|---|---|
| Median age | 67 | 66 |
| Age range | 58-73 | 59-73 |

| | | | | |
|---|---|---|---|---|
| Female | 68 | (31%) | 108 | (49%) |
| Male | 151 | (69%) | 114 | (51%) |
| Smoking_History | | | | |
| Current Smoker | 95 | (44%) | 69 | (31%) |
| Ex Smoker | 97 | (44%) | 98 | (44%) |
| Non Smoker | 26 | (12%) | 55 | (25%) |

| | | |
|---|---|---|
| Unknown | 1 | 0 |

TABLE 16
Demographic status of Test Cohort consisting of lung cancer
cases and control cohorts of individuals with no lung disease

| | | |
|---|---|---|
| | Lung cancer | Normal |
| Demographic | (n = 94) | (n = 96) |

| | | |
|---|---|---|
| Median age | 61 | 60 |
| Age range | 53-67 | 54-67 |

| | | | | |
|---|---|---|---|---|
| Female | 26 | (28%) | 25 | (26%) |
| Male | 68 | (72%) | 71 | (74%) |
| Smoking_ History | | | | |
| Current Smoker | 41 | (49%) | 38 | (40%) |
| Ex Smoker | 31 | (37%) | 35 | (36%) |
| Non Smoker | 12 | (14%) | 23 | (24%) |

| | | |
|---|---|---|
| Unknown | 10 | 0 |

TABLE 17
Demographic status of Validation Cohort consisting of lung cancer
cases and control cohorts of individuals with no lung disease

| | | |
|---|---|---|
| | Lung cancer | Normal |
| Demographic | (n = 205) | (n = 307) |

| | | |
|---|---|---|
| Median age | 66 | 66 |
| Age range | 61-75 | 60-70 |

| | | | | |
|---|---|---|---|---|
| Female | 85 | (41%) | 156 | (51%) |
| Male | 120 | (59%) | 151 | (49%) |
| Smoking_ History | | | | |
| Current Smoker | 29 | (14%) | 109 | (36%) |
| Ex Smoker | 163 | (80%) | 185 | (60%) |
| Non Smoker | 13 | (6.3%) | 10 | (3.3%) |
| Unknown | 0 | (0%) | 3 | (1.0%) |

Optimal cut-offs, in RU, for each cohort's assay results for panels of up to 14 markers were determined using a simulated annealing based multivariate cut-off optimization algorithm. This approach identified a number of different panels of varying sizes (Table 18) for which the performance (Table 19) can be directly compared to the EarlyCDT Lung commercial test performance, that has been determined for the exact same three cohorts of patients.

TABLE 18
Components of panels selected with high specificity

| Panel | Components |
|---|---|
| Panel 1 | p53, SSX1, p62, CAGE, HuD, NY-ESO-1, SOX2, ALDH1, p16, p53-C |
| Panel 2 | p53, SSX1, p62, CAGE, HuD, NY-ESO-1, GBU4-5, p53-C |
| Panel 3 | p53, SSX1, p62, KOC, CAGE, HuD, NY-ESO-1, p16, GBU4-5 |
| Panel 4 | p53, SSX1, p62, KOC, CAGE, HuD, NY-ESO-1, SOX2, p16, α-enolase-1 |
| Panel 5 | p53, SSX1, p62, CAGE, HuD, NY-ESO-1, SOX2, ALDH1, p53-95 |
| Panel 6 | p53, SSX1, p62, CAGE, HuD, NY-ESO-1, ALDH1, p16, α-enolase-1, Lmyc2, p53-C |
| Panel 7 | p53, SSX1, p62, CAGE, HuD, NY-ESO-1, SOX2, p16, p53-C |
| Panel 8 | p53, SSX1, p62, CAGE, NY-ESO-1, p16, p53-95, p53-C |
| Panel 9 | p53, SSX1, p62, NY-ESO-1, SOX2, α-enolase-1, p53-C |
| Panel 10 | p53, SSX1, p62, KOC, HuD, NY-ESO-1, SOX2, α-enolase-1, p53-C |
| Panel 11 | p53, SSX1, KOC, CAGE, HuD, NY-ESO-1, SOX2, ALDH1, p16 |
| Panel 12 | p53, SSX1, p62, NY-ESO-1, SOX2, ALDH1, p16, p53-95 |
| Panel 13 | p53, SSX1, KOC, HuD, NY-ESO-1, SOX2, p16, GBU4-5, p53-95 |
| Panel 14 | p53, SSX1, KOC, CAGE, HuD, SOX2, GBU4-5, α-enolase-1, Lmyc2, p53-C |
| Panel 15 | p53, SSX1, KOC, CAGE, HuD, p16, GBU4-5, p53-95 |
| Panel 16 | p53, SSX1, KOC, CAGE, SOX2, ALDH1, GBU4-5, Lmyc2 |
| Panel 17 | p53, SSX1, p62, KOC, CAGE, SOX2, α-enolase-1, p53-C |

TABLE 19
Summary of cohort performance in selected high specificity panels

| Panel | sens tr | spec tr | sens te | spec te | sens val | spec val | sens all | spec all |
|---|---|---|---|---|---|---|---|---|
| EarlyCDT | 28.8% | 90.5% | 22.6% | 92.7% | 37.4% | 87.4% | 31.1% | 89.3% |
| Panel 1 | 35.2% | 96.8% | 32.3% | 94.8% | 33.7% | 85.3% | 34.0% | 90.9% |
| Panel 2 | 34.7% | 96.4% | 29.0% | 93.8% | 30.7% | 84.7% | 32.1% | 90.2% |
| Panel 3 | 37.4% | 95.0% | 37.6% | 91.7% | 35.6% | 82.1% | 36.8% | 88.2% |
| Panel 4 | 32.4% | 97.3% | 28.0% | 96.9% | 31.7% | 89.9% | 31.3% | 93.6% |
| Panel 5 | 37.0% | 95.0% | 32.3% | 91.7% | 34.6% | 81.8% | 35.2% | 88.0% |
| Panel 6 | 32.9% | 96.8% | 32.3% | 93.8% | 33.2% | 86.3% | 32.9% | 91.2% |
| Panel 7 | 39.3% | 93.2% | 34.4% | 89.6% | 35.1% | 85.3% | 36.8% | 88.8% |
| Panel 8 | 33.8% | 95.5% | 28.0% | 92.7% | 32.2% | 83.7% | 32.1% | 89.3% |
| Panel 9 | 31.5% | 95.9% | 26.9% | 92.7% | 29.3% | 88.6% | 29.8% | 91.8% |
| Panel 10 | 31.5% | 95.9% | 25.8% | 92.7% | 28.8% | 88.6% | 29.4% | 91.8% |
| Panel 11 | 34.2% | 94.6% | 32.3% | 92.7% | 34.6% | 85.3% | 34.0% | 89.8% |
| Panel 12 | 37.0% | 92.8% | 34.4% | 89.6% | 35.1% | 80.8% | 35.8% | 86.4% |
| Panel 13 | 33.8% | 92.3% | 29.0% | 89.6% | 37.1% | 79.5% | 34.2% | 85.6% |
| Panel 14 | 31.5% | 93.2% | 28.0% | 91.7% | 35.6% | 81.4% | 32.5% | 87.2% |
| Panel 15 | 32.4% | 92.8% | 26.9% | 89.6% | 30.2% | 83.7% | 30.6% | 87.8% |
| Panel 16 | 30.6% | 93.7% | 30.1% | 90.6% | 30.2% | 83.1% | 30.4% | 88.0% |
| Panel 17 | 36.5% | 90.5% | 33.3% | 86.5% | 38.5% | 78.5% | 36.8% | 84.0% |

sens = sensitivity; spec = specificity; tr = training cohort; te = test cohort; val = validation cohort

These results suggest that panels of 6-10 markers incorporating at least p53, SSX1 and p62 and/or KOC in each, result in a similar performance to the EarlyCDT Lung test panel in each of the three Western cohorts investigated. It is no surprise that panels can have autoantibodies to p62 and/or KOC, as p62 and KOC are both members of the highly conserved family of insulin-like growth factor 2 mRNA-binding (IMP) proteins and are 65% homologous and therefore these proteins are highly likely to share tumour associated autoantibody epitopes. Whilst in the Western population, panels incorporating at least p53, SSX1 and p62 and/or KOC in each, have similar performance to the EarlyCDT Lung test panel, the enhanced performance for a Chinese population for the detection of lung cancer provides panels for tests that are applicable globally.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications and patent applications are cited herein, the disclosures of which are incorporated by reference in their entireties.

The invention may further be understood with reference to the following clauses:

1. A method of detecting lung cancer in a mammalian subject by detecting three or more autoantibodies in a test sample comprising a bodily fluid from the mammalian subject, wherein three of the autoantibodies are immunologically specific for the tumour marker antigens p53, p62, and SSX1, and wherein the method comprises the steps of:
(a) contacting the test sample with a panel of three or more tumour marker antigens of which three of the tumour marker antigens are p53, p62, and SSX1; and
(b) determining the presence or absence of complexes of the tumour marker antigens bound to autoantibodies present in the test sample,
wherein the presence of complexes containing at least p53, p62 and SSX1 is indicative of the presence of lung cancer.
   2. The method of clause 1, wherein the panel of three or more tumour marker antigens comprises p53, p62, and SSX1 and one or more tumour marker antigens selected from the group consisting of HuD, MAGE A4, SOX2, NY-ESO-1, CAGE, CK20, GBU4-5, p53-95, CK8, KRAS, and α-enolase-1.
   3. The method of clause 1 or clause 2, wherein four or more autoantibodies are detected, wherein the method comprises the step of (a) contacting the test sample with a panel of four or more tumour marker antigens of which four of the tumour marker antigens are p53, p62, SSX1 and HuD, and wherein the presence of complexes containing at least p53, p62, SSX1 and HuD is indicative of the presence of lung cancer.
   4. The method of clause 1 or clause 2, wherein five or more autoantibodies are detected, wherein the method comprises the step of (a) contacting the test sample with a panel of five or more tumour marker antigens of which five of the tumour marker antigens are p53, p62, SSX1, HuD and MAGE A4, and wherein the presence of complexes containing at least p53, p62, SSX1, HuD and MAGE A4 is indicative of the presence of lung cancer.
   5. The method of clause 4, wherein the panel of five or more tumour marker antigens comprises p53, p62, SSX1, HuD, and MAGE A4, and one or more tumour marker antigens selected from the group consisting of SOX2, NY-ESO-1, CAGE, CK20, GBU4-5, p53-95, CK8 and KRAS.
   6. The method of clause 5, wherein the panel of five or more tumour marker antigens comprises or consists of one of the groups of tumour marker antigens selected from:
   (i) p53, p62, SSX1, HuD, MAGE A4, SOX2, CAGE;
   (ii) p53, p62, SSX1, HuD, MAGE A4, SOX2, NY-ESO-1, CK20;
   (iii) p53, p62, SSX1, HuD, MAGE A4, SOX2, NY-ESO-1, CAGE;
   (iv) p53, p62, SSX1, HuD, MAGE A4, SOX2, CAGE, CK20;
   (v) p53, p62, SSX1, HuD, MAGE A4, NY-ESO-1, CAGE, CK20;
   (vi) p53, p62, SSX1, HuD, MAGE A4, SOX2, NY-ESO-1, CAGE, CK20;
   (vii) p53, p62, SSX1, HuD, MAGE A4, SOX2, CK20, CK8, KRAS;
   (viii) p53, p62, SSX1, HuD, MAGE A4, SOX2, NY-ESO-1, CK20, CK8, P53-95, KRAS;
   (ix) p53, p62, SSX1, HuD, MAGE A4, SOX2, NY-ESO-1, CAGE, CK20, CK8, KRAS; and
   (x) p53, p62, SSX1, HuD, MAGE A4, SOX2, NY-ESO-1, CAGE, GBU4-5, CK8, KRAS.
   7. The method of any one of the preceding clauses, further comprising the step of:
(c) detecting the amount of specific binding between the tumour marker antigen and autoantibodies present in the test sample,
   wherein the presence or absence of the autoantibody is based upon a comparison between the amount of specific binding observed and a pre-determined cut-off value.
   8. The method of any one of the preceding clauses, wherein the tumour marker antigen is provided in a plurality of different amounts, and wherein the method comprises the steps of:
(a) contacting the test sample with a plurality of different amounts of the tumour marker antigen;
(b) determining the presence or absence of complexes of the tumour marker antigen bound to autoantibodies present in the test sample;
(c) detecting the amount of specific binding between the tumour marker antigen and the autoantibodies;
(d) plotting or calculating a curve of the amount of the specific binding versus the amount of tumour marker antigen for each amount of tumour marker antigen used in step (a); and
(e) determining the presence or absence of the autoantibody based upon the amount of specific binding between the tumour marker antigen and the autoantibody at each different amount of tumour marker antigen used.
   9. The method of clause 8, wherein the method further comprises the steps of:
(d1) calculating a secondary curve parameter from the curve plotted or calculated in step (d); and
(e) determining the presence or absence of the autoantibody based upon a combination of:
(i) the amount of specific binding between the autoantibody and the tumour marker antigen determined in step (b); and
(ii) the secondary curve parameter determined in step (d1).
   10. An in vitro method of determining an autoantibody profile of an individual suffering from lung cancer by detecting three or more autoantibodies in a test sample comprising a bodily fluid from the mammalian subject, wherein three of the autoantibodies are immunologically specific for the tumour marker antigens p53, p62 and SSX1, which method comprises the steps of:
a) contacting the test sample with a panel of three or more tumour marker antigens of which three of the tumour marker antigens are p53, p62, and SSX1; and
b) determining the presence or absence of complexes of the tumour marker antigens bound to autoantibodies present in the test sample, wherein the method is repeated to build up a profile of autoantibody production.
   11. A method of diagnosing and treating lung cancer in a mammalian subject by detecting three or more autoantibodies in a test sample comprising a bodily fluid from the mammalian subject, wherein three of the autoantibodies are immunologically specific for the tumour marker antigens p53, p62 and SSX1, which method comprises the steps of:
(a) contacting the test sample with a panel of three or more tumour marker antigens of which three of the tumour marker antigens are p53, p62, and SSX1;
(b) determining the presence or absence of complexes of the tumour marker antigens bound to autoantibodies present in the test sample;
(c) diagnosing the subject with lung cancer when complexes containing at least the tumour marker antigens p53, p62 and SSX1 bound to autoantibodies present in the test sample are detected; and
(d) administering a lung cancer treatment to the diagnosed subject.
   12. A method of predicting response to a lung cancer treatment, the method comprising detecting three or more autoantibodies in a test sample comprising a bodily fluid from a mammalian subject, wherein three of the autoantibodies are immunologically specific for the tumour marker antigens p53, p62 and SSX1, which method comprises the steps of:
(a) contacting the test sample with a panel of three or more tumour marker antigens of which three of the tumour marker antigens are p53, p62, and SSX1;
(b) determining the presence or absence of complexes of the tumour marker antigens bound to autoantibodies present in the test sample;
(c) detecting the amount of specific binding between the tumour marker antigens and autoantibodies present in the test sample; and
(d) comparing the amount of specific binding between the tumour marker antigens and the autoantibodies with a previously established relationship between the amount of binding and the likely outcome of treatment,
   wherein a change in the amount of specific binding, when compared to controls, predicts that the patient will or will not respond to the lung cancer treatment.
   13. The method of clause 11 or clause 12, wherein the lung cancer treatment is selected from the group consisting of surgery, video-assisted thoracoscopic surgery, radiotherapy, chemotherapy, immunotherapy, radiofrequency ablation, biological therapy, cryotherapy and photodynamic therapy.
   14. Use of a panel of three or more tumour marker antigens for the detection of lung cancer in a mammalian subject by detecting autoantibodies immunologically specific for p53, p62 and SSX1 in a test sample comprising a bodily fluid from the mammalian subject.
   15. The method of any one of clauses 10-13 or the use of clause 14, wherein the panel of three or more tumour marker antigens comprises p53, p62, and SSX1 and one or more tumour marker antigens selected from the group consisting of HuD, MAGE A4, SOX2, NY-ESO-1, CAGE, CK20, GBU4-5, p53-95, CK8, KRAS, and α-enolase-1.
   16. The method of any one of clauses 10-13 or the use of clause 14, wherein four or more autoantibodies are detected, wherein the method or use comprises contacting the test sample with a panel of four or more tumour marker antigens of which four of the tumour marker antigens are p53, p62, SSX1, and HuD, and wherein the presence of at least complexes containing p53, p62, SSX1, and HuD is detected.
   17. The method of any one of clauses 10-13 or the use of clause 14, wherein five or more autoantibodies are detected, wherein the method or use comprises contacting the test sample with a panel of five or more tumour marker antigens of which five of the tumour marker antigens are p53, p62, SSX1, HuD and MAGE A4, and wherein the presence of at least complexes containing p53, p62, SSX1, HuD and MAGE A4 is detected.
   18. The method or use of clause 17, wherein the panel of five or more tumour marker antigens comprises p53, p62, SSX1, HuD, and MAGE A4, and one or more tumour marker antigens selected from the group consisting of SOX2, NY-ESO-1, CAGE, CK20, GBU4-5, p53-95, CK8, and KRAS.
   19. The method or use of clause 18, wherein the panel of five or more tumour marker antigens comprises or consists of one of the groups of tumour marker antigens selected from:
   (i) p53, p62, SSX1, HuD, MAGE A4, SOX2, CAGE;
   (ii) p53, p62, SSX1, HuD, MAGE A4, SOX2, NY-ESO-1, CK20;
   (iii) p53, p62, SSX1, HuD, MAGE A4, SOX2, NY-ESO-1, CAGE;
   (iv) p53, p62, SSX1, HuD, MAGE A4, SOX2, CAGE, CK20;
   (v) p53, p62, SSX1, HuD, MAGE A4, NY-ESO-1, CAGE, CK20;
   (vi) p53, p62, SSX1, HuD, MAGE A4, SOX2, NY-ESO-1, CAGE, CK20;
   (vii) p53, p62, SSX1, HuD, MAGE A4, SOX2, CK20, CK8, KRAS;
   (viii) p53, p62, SSX1, HuD, MAGE A4, SOX2, NY-ESO-1, CK20, CK8, p53-95, KRAS;
   (ix) p53, p62, SSX1, HuD, MAGE A4, SOX2, NY-ESO-1, CAGE, CK20, CK8, KRAS; and
   (x) p53, p62, SSX1, HuD, MAGE A4, SOX2, NY-ESO-1, CAGE, GBU4-5, CK8, KRAS.
   20. A kit for the detection of autoantibodies in a test sample comprising a bodily fluid from a mammalian subject comprising:
   (a) a panel of three or more tumour marker antigens of which three of the tumour marker antigens are p53, p62, and SSX1; and
   (b) a reagent capable of detecting complexes of the tumour marker antigens bound to autoantibodies present in the test sample.
   21. The kit of clause 20, further comprising:
   (c) means for contacting the tumour marker antigens with a test sample comprising a bodily fluid from a mammalian subject.
   22. The kit of clause 21, wherein the means for contacting the tumour marker antigens with a test sample comprising a bodily fluid from a mammalian subject comprises the tumour marker antigens immobilised on a chip, slide, plate, wells of a microtitre plate, bead, membrane or nanoparticle.
   23. The kit of any one of clauses 20-22, wherein the panel of three or more tumour marker antigens comprises p53, p62, and SSX1 and one or more tumour marker antigens selected from the group consisting of HuD, MAGE A4, SOX2, NY-ESO-1, CAGE, CK20, GBU4-5, p53-95, CK8, KRAS, and α-enolase-1.
   24. The kit of any one of clauses 20-22, comprising a panel of four or more tumour marker antigens of which four of the tumour marker antigens are p53, p62, SSX1 and HuD.
   25. The kit of any one of clauses 20-22, comprising a panel of five or more tumour marker antigens of which five of the tumour marker antigens are p53, p62, SSX1, HuD, and MAGE A4.
   26. The kit of clause 25, wherein the panel of five or more tumour marker antigens comprises p53, p62, SSX1, HuD and MAGE A4, and one or more tumour marker antigens selected from the group consisting of SOX2, NY-ESO-1, CAGE, CK20, GBU4-5, p53-95, CK8 and KRAS.
   27. The kit of clause 26, wherein the panel of five or more tumour marker antigens comprises or consists of one of the groups of tumour marker antigens selected from:
   (i) p53, p62, SSX1, HuD, MAGE A4, SOX2, CAGE;
   (ii) p53, p62, SSX1, HuD, MAGE A4, SOX2, NY-ESO-1, CK20;
   (iii) p53, p62, SSX1, HuD, MAGE A4, SOX2, NY-ESO-1, CAGE;
   (iv) p53, p62, SSX1, HuD, MAGE A4, SOX2, CAGE, CK20;
   (v) p53, p62, SSX1, HuD, MAGE A4, NY-ESO-1, CAGE, CK20;
   (vi) p53, p62, SSX1, HuD, MAGE A4, SOX2, NY-ESO-1, CAGE, CK20;
   (vii) p53, p62, SSX1, HuD, MAGE A4, SOX2, CK20, CK8, KRAS;
   (viii) p53, p62, SSX1, HuD, MAGE A4, SOX2, NY-ESO-1, CK20, CK8, p53-95, KRAS;
   (ix) p53, p62, SSX1, HuD, MAGE A4, SOX2, NY-ESO-1, CAGE, CK20, CK8, KRAS; and
   (x) p53, p62, SSX1, HuD, MAGE A4, SOX2, NY-ESO-1, CAGE, GBU4-5, CK8, KRAS.
   28. The kit of any one of clauses 20-27 for the detection of lung cancer.
   29. The method, use or kit of any one of the preceding clauses, wherein the tumour marker antigen is a naturally occurring protein or polypeptide, a recombinant protein or polypeptide, a synthetic protein or polypeptide, a synthetic peptide, a peptide mimetic, a polysaccharide or a nucleic acid.
   30. The method, use or kit of any one of the preceding clauses, wherein the bodily fluid is selected from the group consisting of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears and wound drainage fluid.
   31. A method of detecting lung cancer in a mammalian subject by detecting an autoantibody in a test sample comprising a bodily fluid from the mammalian subject, wherein the autoantibody is immunologically specific for a tumour marker antigen selected from the group consisting of p53, SSX1, SOX2, GBU4-5, HuD, p53-95 and CK8, and wherein the method comprises the steps of:
(a) contacting the test sample with a tumour marker antigen selected from the group consisting of p53, SSX1, SOX2, GBU4-5, HuD, p53-95 and CK8; and
(b) determining the presence or absence of complexes of the tumour marker antigen bound to autoantibodies present in the test sample, wherein the presence of said complexes is indicative of the presence of lung cancer.
   32. The method of clause 31, wherein two, three, four, five, six, seven or more autoantibodies are detected, and the method comprises the step of
(a) contacting the test sample with a panel of two or more, three or more, four or more, five or more, six or more or seven or more tumour marker antigens wherein at least two, at least three, at least four, at least five, at least six or seven of the tumour marker antigens are selected from the group consisting of p53, SSX1, SOX2, GBU4-5, HuD, p53-95 and CK8, wherein the presence of complexes containing at least two, at least three, at least four, at least five, at least six or seven of the tumour marker antigens selected from the group consisting of p53, SSX1, SOX2, GBU4-5, HuD, p53-95 and CK8 is indicative of the presence of lung cancer.
   33. The method of clause 31, wherein seven or more autoantibodies are detected, and the method comprises the step of (a) contacting the test sample with a panel of seven or more tumour marker antigens of which seven of the tumour marker antigens are p53, SSX1, SOX2, GBU4-5, HuD, p53-95 and CK8,
wherein the presence of complexes containing at least one, at least two, at least three, at least four, at least five, at least six tumour marker antigens selected from the group consisting of p53, SSX1, SOX2, GBU4-5, HuD, p53-95 and CK8 is indicative of the presence of lung cancer.
   34. The method of clause 33, wherein the presence of complexes containing at least p53, SSX1, SOX2, GBU4-5, HuD, p53-95 and CK8 is indicative of the presence of lung cancer.
   35. A kit for the detection of autoantibodies in a test sample comprising a bodily fluid from a mammalian subject comprising:
(a) a panel of two or more, three or more, four or more, five or more, six or more, seven or more tumour marker antigens of which at least two, at least three, at least four, at least five, at least six or seven of the tumour marker antigens are selected from the group consisting of p53, SSX1, SOX2, GBU4-5, HuD, p53-95 and CK8; and
(b) a reagent capable of detecting complexes of the tumour marker antigens bound to autoantibodies present in the test sample.
   36. A kit for the detection of autoantibodies in a test sample comprising a bodily fluid from a mammalian subject comprising:
(a) a panel of seven or more tumour marker antigens of which seven of the tumour marker antigens are p53, SSX1, SOX2, GBU4-5, HuD, p53-95 and CK8; and
(b) a reagent capable of detecting complexes of the tumour marker antigens bound to autoantibodies present in the test sample.
   37. Use of a panel of two or more, three or more, four or more, five or more, six or more or seven or more tumour marker antigens for the detection of lung cancer in a mammalian subject, by detecting autoantibodies immunologically specific for two or more, three or more, four or more, five or more, six or more or seven tumour marker antigens selected from the group consisting of p53, SSX1, SOX2, GBU4-5, HuD, p53-95 and CK8, in a test sample comprising a bodily fluid from the mammalian subject.

1. A method of detecting lung cancer in a mammalian subject by detecting three or more autoantibodies in a test sample comprising a bodily fluid from the mammalian subject, wherein three of the autoantibodies are immunologically specific for the tumour marker antigens p53, SSX1 and either p62 or KOC, and wherein the method comprises the steps of:
(a) contacting the test sample with a panel of three or more tumour marker antigens of which three of the tumour marker antigens are p53, SSX1, and either p62 or KOC; and
(b) determining the presence or absence of complexes of the tumour marker antigens bound to autoantibodies present in the test sample,
wherein the presence of complexes containing at least p53, SSX1, and either p62 or KOC is indicative of the presence of lung cancer.

2. The method of claim 1, wherein the panel of three or more tumour marker antigens comprises p53, SSX1, and p62, and the presence of complexes containing at least p53, SSX1, and p62 is indicative of the presence of lung cancer.

3. The method of claim 1, wherein the panel of three or more tumour marker antigens comprises p53, SSX1, and KOC, and the presence of complexes containing at least p53, SSX1, and KOC is indicative of the presence of lung cancer.

4. The method of claim 1, wherein the panel of three or more tumour marker antigens comprises p53, SSX1, and p62 and/or KOC, and one or more tumour marker antigens selected from the group consisting of HuD, MAGE A4, SOX2, NY-ESO-1, CAGE, CK20, GBU4-5, p53-95, CK8, KRAS-G13C/Q61H, ALDH1, p16, Lmyc2, and α-enolase-1.

5. The method claim 1, wherein four or more autoantibodies are detected, wherein the method comprises the step of (a) contacting the test sample with a panel of four or more tumour marker antigens of which four of the tumour marker antigens are p53, SSX1, p62 or KOC, and HuD, and wherein the presence of complexes containing at least p53, SSX1, p62 or KOC, and HuD is indicative of the presence of lung cancer.

6. The method of claim 1, wherein five or more autoantibodies are detected, wherein the method comprises the step of (a) contacting the test sample with a panel of five or more tumour marker antigens of which five of the tumour marker antigens are p53, SSX1, p62 or KOC, HuD, and MAGE A4, and wherein the presence of complexes containing at least p53, SSX1, p62 or KOC, HuD and MAGE A4 is indicative of the presence of lung cancer.

7. The method of claim 6, wherein the panel of five or more tumour marker antigens comprises p53, SSX1, p62 and/or KOC, HuD, and MAGE A4, and one or more tumour marker antigens selected from the group consisting of SOX2, NY-ESO-1, CAGE, CK20, GBU4-5, p53-95, CK8, KRAS-G13C/Q61H, ALDH1, p16, Lmyc2, and α-enolase-1.

8. The method of claim 1, wherein the panel of tumour marker antigens comprises or consists of one of the groups of tumour marker antigens selected from:
(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS-G13C/Q61H;
(viii) p53, SSX1, p62, HuD, MAGE A4, SOX2, NY-ESO-1, CK20, CK8, P53-95, KRAS-G13C/Q61H;
(ix) p53, SSX1, p62, HuD, MAGE A4, SOX2, NY-ESO-1, CAGE, CK20, CK8, KRAS-G13C/Q61H;
(x) p53, SSX1, p62, HuD, MAGE A4, SOX2, NY-ESO-1, CAGE, GBU4-5, CK8, KRAS-G13C/Q61H;
(xi) p53, SSX1, p62, CAGE, HuD, NY-ESO-1, SOX2, ALDH1, p16;
(xii) p53, SSX1, p62, CAGE, HuD, NY-ESO-1, GBU4-5;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1;
(xvi) p53, SSX1, p62, CAGE, HuD, NY-ESO-1, ALDH1, p16, α-enolase-1, Lmyc2;
(xvii) p53, SSX1, p62, CAGE, HuD, NY-ESO-1, SOX2, p16;
(xviii) p53, SSX1, p62, CAGE, NY-ESO-1, p16;
(xix) p53, SSX1, p62, NY-ESO-1, SOX2, α-enolase-1;
(xx) p53, SSX1, p62, KOC, HuD, NY-ESO-1, SOX2, α-enolase-1;
(xxi) p53, SSX1, p62, NY-ESO-1, SOX2, ALDH1, p16;
(xxii) p53, SSX1, p62, KOC, CAGE, SOX2, α-enolase-1;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5;
(xxv) p53, SSX1, KOC, CAGE, HuD, SOX2, GBU4-5, α-enolase-1, Lmyc2;
(xxvi) p53, SSX1, KOC, CAGE, HuD, p16, GBU4-5; and
(xxvii) p53, SSX1, KOC, CAGE, SOX2, ALDH1, GBU4-5, Lmyc2.

9. The method of claim 1, further comprising the step of:
(c) detecting the amount of specific binding between the tumour marker antigen and autoantibodies present in the test sample,
wherein the presence or absence of the autoantibody is based upon a comparison between the amount of specific binding observed and a pre-determined cut-off value.

10. The method of claim 1, wherein the tumour marker antigen is provided in a plurality of different amounts, and wherein the method comprises the steps of:
(a) contacting the test sample with a plurality of different amounts of the tumour marker antigen;
(b) determining the presence or absence of complexes of the tumour marker antigen bound to autoantibodies present in the test sample;
(c) detecting the amount of specific binding between the tumour marker antigen and the autoantibodies;
(d) plotting or calculating a curve of the amount of the specific binding versus the amount of tumour marker antigen for each amount of tumour marker antigen used in step (a); and
(e) determining the presence or absence of the autoantibody based upon the amount of specific binding between the tumour marker antigen and the autoantibody at each different amount of tumour marker antigen used.

11. The method of claim 10, wherein the method further comprises the steps of:
(d1) calculating a secondary curve parameter from the curve plotted or calculated in step (d); and
(e) determining the presence or absence of the autoantibody based upon a combination of:
(i) the amount of specific binding between the autoantibody and the tumour marker antigen determined in step (b); and
(ii) the secondary curve parameter determined in step (d1).

12. An in vitro method of determining an autoantibody profile of an individual suffering from lung cancer by detecting three or more autoantibodies in a test sample comprising a bodily fluid from the mammalian subject, wherein three of the autoantibodies are immunologically specific for the tumour marker antigens p53, SSX1, and either p62 or KOC, which method comprises the steps of:
a) contacting the test sample with a panel of three or more tumour marker antigens of which three of the tumour marker antigens are p53, SSX1, and either p62 or KOC; and
b) determining the presence or absence of complexes of the tumour marker antigens bound to autoantibodies present in the test sample, wherein the method is repeated to build up a profile of autoantibody production.

13. A method of diagnosing and treating lung cancer in a mammalian subject by detecting three or more autoantibodies in a test sample comprising a bodily fluid from the mammalian subject, wherein three of the autoantibodies are immunologically specific for the tumour marker antigens p53, SSX1, and either p62 or KOC, which method comprises the steps of:
(a) contacting the test sample with a panel of three or more tumour marker antigens of which three of the tumour marker antigens are p53, SSX1, and either p62 or KOC;
(b) determining the presence or absence of complexes of the tumour marker antigens bound to autoantibodies present in the test sample;
(c) diagnosing the subject with lung cancer when complexes containing at least the tumour marker antigens p53, SSX1, and either p62 or KOC bound to autoantibodies present in the test sample are detected; and
(d) administering a lung cancer treatment to the diagnosed subject.

14. A method of predicting response to a lung cancer treatment, the method comprising detecting three or more autoantibodies in a test sample comprising a bodily fluid from a mammalian subject, wherein three of the autoantibodies are immunologically specific for the tumour marker antigens p53, SSX1, and either p62 or KOC, which method comprises the steps of:
(a) contacting the test sample with a panel of three or more tumour marker antigens of which three of the tumour marker antigens are p53, SSX1, and either p62 or KOC;
(b) determining the presence or absence of complexes of the tumour marker antigens bound to autoantibodies present in the test sample;
(c) detecting the amount of specific binding between the tumour marker antigens and autoantibodies present in the test sample; and
(d) comparing the amount of specific binding between the tumour marker antigens and the autoantibodies with a previously established relationship between the amount of binding and the likely outcome of treatment,
wherein a change in the amount of specific binding, when compared to controls, predicts that the patient will or will not respond to the lung cancer treatment.

15. The method of claim 13, wherein the lung cancer treatment is selected from the group consisting of surgery, video-assisted thoracoscopic surgery, radiotherapy, chemotherapy, immunotherapy, radiofrequency ablation, biological therapy, cryotherapy and photodynamic therapy.

16. Use of a panel of three or more tumour marker antigens for the detection of lung cancer in a mammalian subject by detecting autoantibodies immunologically specific for p53, SSX1, and either p62 or KOC, in a test sample comprising a bodily fluid from the mammalian subject.

17. The method of claim 12, wherein the panel of three or more tumour marker antigens comprises p53, SSX1, and p62.

18. The method of claim 12, wherein the panel of three or more tumour marker antigens comprises p53, SSX1, and KOC.

19. The method of claim 12, wherein the panel of three or more tumour marker antigens comprises p53, SSX1, and p62 and/or KOC, and one or more tumour marker antigens selected from the group consisting of HuD, MAGE A4, SOX2, NY-ESO-1, CAGE, CK20, GBU4-5, p53-95, CK8, KRAS-G13C/Q61H, ALDH1, p16, Lmyc2, and α-enolase-1.

20. The method of claim 12, wherein four or more autoantibodies are detected, wherein the method or use comprises contacting the test sample with a panel of four or more tumour marker antigens of which four of the tumour marker antigens are p53, SSX1, p62 or KOC, and HuD, and wherein the presence of at least complexes containing p53, SSX1, p62 or KOC, and HuD is detected.

21. The method of claim 12, wherein five or more autoantibodies are detected, wherein the method or use comprises contacting the test sample with a panel of five or more tumour marker antigens of which five of the tumour marker antigens are p53, SSX1, p62 or KOC, HuD and MAGE A4, and wherein the presence of at least complexes containing p53, SSX1, p62 or KOC, HuD and MAGE A4 is detected.

22. The method or use of claim 21, wherein the panel of five or more tumour marker antigens comprises p53, SSX1, p62 and/or KOC, HuD, and MAGE A4, and one or more tumour marker antigens selected from the group consisting of SOX2, NY-ESO-1, CAGE, CK20, GBU4-5, p53-95, CK8, KRAS-G13C/Q61H, ALDH1, p16, Lmyc2, and α-enolase-1.

23. The method of claim 12, wherein the panel of tumour marker antigens comprises or consists of one of the groups of tumour marker antigens selected from:
(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS-G13C/Q61H;
(viii) p53, SSX1, p62, HuD, MAGE A4, SOX2, NY-ESO-1, CK20, CK8, p53-95, KRAS-G13C/Q61H;
(ix) p53, SSX1, p62, HuD, MAGE A4, SOX2, NY-ESO-1, CAGE, CK20, CK8, KRAS-G13C/Q61H;
(x) p53, SSX1, p62, HuD, MAGE A4, SOX2, NY-ESO-1, CAGE, GBU4-5, CK8, KRAS-G13C/Q61H;
(xi) p53, SSX1, p62, CAGE, HuD, NY-ESO-1, SOX2, ALDH1, p16;
(xii) p53, SSX1, p62, CAGE, HuD, NY-ESO-1, GBU4-5;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1;
(xvi) p53, SSX1, p62, CAGE, HuD, NY-ESO-1, ALDH1, p16, α-enolase-1, Lmyc2;
(xvii) p53, SSX1, p62, CAGE, HuD, NY-ESO-1, SOX2, p16;
(xviii) p53, SSX1, p62, CAGE, NY-ESO-1, p16;
(xix) p53, SSX1, p62, NY-ESO-1, SOX2, α-enolase-1;
(xx) p53, SSX1, p62, KOC, HuD, NY-ESO-1, SOX2, α-enolase-1;
(xxi) p53, SSX1, p62, NY-ESO-1, SOX2, ALDH1, p16;
(xxii) p53, SSX1, p62, KOC, CAGE, SOX2, α-enolase-1;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5;
(xxv) p53, SSX1, KOC, CAGE, HuD, SOX2, GBU4-5, α-enolase-1, Lmyc2;
(xxvi) p53, SSX1, KOC, CAGE, HuD, p16, GBU4-5; and
(xxvii) p53, SSX1, KOC, CAGE, SOX2, ALDH1, GBU4-5, Lmyc2.

24. A kit for the detection of autoantibodies in a test sample comprising a bodily fluid from a mammalian subject comprising:
(a) a panel of three or more tumour marker antigens of which three of the tumour marker antigens are p53, SSX1, and either p62 or KOC; and
(b) a reagent capable of detecting complexes of the tumour marker antigens bound to autoantibodies present in the test sample.

25. The kit of claim 24, wherein the panel of three or more tumour marker antigens comprises p53, SSX1, and p62.

26. The kit of claim 24, wherein the panel of three or more tumour marker antigens comprises p53, SSX1, and KOC.

27. The kit of claim 24, further comprising:
(c) means for contacting the tumour marker antigens with a test sample comprising a bodily fluid from a mammalian subject.

28. The kit of claim 27, wherein the means for contacting the tumour marker antigens with a test sample comprising a bodily fluid from a mammalian subject comprises the tumour marker antigens immobilised on a chip, slide, plate, wells of a microtitre plate, bead, membrane or nanoparticle.

29. The kit of claim 24, wherein the panel of three or more tumour marker antigens comprises p53, SSX1, and p62 and/or KOC and one or more tumour marker antigens selected from the group consisting of HuD, MAGE A4, SOX2, NY-ESO-1, CAGE, CK20, GBU4-5, p53-95, CK8, KRAS-G13C/Q61H, ALDH1, p16, Lmyc2, and α-enolase-1.

30. The kit of claim 24, comprising a panel of four or more tumour marker antigens of which four of the tumour marker antigens are p53, SSX1, p62 or KOC, and HuD.

31. The kit of claim 24, comprising a panel of five or more tumour marker antigens of which five of the tumour marker antigens are p53, SSX1, p62 or KOC, HuD, and MAGE A4.

32. The kit of claim 31, wherein the panel of five or more tumour marker antigens comprises p53, SSX1, p62 and/or KOC, HuD and MAGE A4, and one or more tumour marker antigens selected from the group consisting of SOX2, NY-ESO-1, CAGE, CK20, GBU4-5, p53-95, CK8, KRAS-G13C/Q61H, ALDH1, p16, Lmyc2, and α-enolase-1.

33. The kit of claim 31, wherein the panel of tumour marker antigens comprises or consists of one of the groups of tumour marker antigens selected from:
(i) p53, SSX1, p62, HuD, MAGE A4, SOX2, CAGE;
(ii) p53, SSX1, p62, HuD, MAGE A4, SOX2, NY-ESO-1, CK20;
(iii) p53, SSX1, p62, HuD, MAGE A4, SOX2, NY-ESO-1, CAGE;
(iv) p53, SSX1, p62, HuD, MAGE A4, SOX2, CAGE, CK20;
(v) p53, SSX1, p62, HuD, MAGE A4, NY-ESO-1, CAGE, CK20;
(vi) p53, SSX1, p62, HuD, MAGE A4, SOX2, NY-ESO-1, CAGE, CK20;
(vii) p53, SSX1, p62, HuD, MAGE A4, SOX2, CK20, CK8, KRAS-G13C/Q61H;
(viii) p53, SSX1, p62, HuD, MAGE A4, SOX2, NY-ESO-1, CK20, CK8, p53-95, KRAS-G13C/Q61H;
(ix) p53, SSX1, p62, HuD, MAGE A4, SOX2, NY-ESO-1, CAGE, CK20, CK8, KRAS-G13C/Q61H;
(x) p53, SSX1, p62, HuD, MAGE A4, SOX2, NY-ESO-1, CAGE, GBU4-5, CK8, KRAS-G13C/Q61H;
(xi) p53, SSX1, p62, CAGE, HuD, NY-ESO-1, SOX2, ALDH1, p16;
(xii) p53, SSX1, p62, CAGE, HuD, NY-ESO-1, GBU4-5;
(xiii) p53, SSX1, p62, KOC, CAGE, HuD, NY-ESO-1, p16, GBU4-5;
(xiv) p53, SSX1, p62, KOC, CAGE, HuD, NY-ESO-1, SOX2, p16, α-enolase-1;
(xv) p53, SSX1, p62, CAGE, HuD, NY-ESO-1, SOX2, ALDH1;
(xvi) p53, SSX1, p62, CAGE, HuD, NY-ESO-1, ALDH1, p16, α-enolase-1, Lmyc2;
(xvii) p53, SSX1, p62, CAGE, HuD, NY-ESO-1, SOX2, p16;
(xviii) p53, SSX1, p62, CAGE, NY-ESO-1, p16;
(xix) p53, SSX1, p62, NY-ESO-1, SOX2, α-enolase-1;
(xx) p53, SSX1, p62, KOC, HuD, NY-ESO-1, SOX2, α-enolase-1;
(xxi) p53, SSX1, p62, NY-ESO-1, SOX2, ALDH1, p16;
(xxii) p53, SSX1, p62, KOC, CAGE, SOX2, α-enolase-1;
(xxiii) p53, SSX1, KOC, CAGE, HuD, NY-ESO-1, SOX2, ALDH1, p16;
(xxiv) p53, SSX1, KOC, HuD, NY-ESO-1, SOX2, p16, GBU4-5;
(xxv) p53, SSX1, KOC, CAGE, HuD, SOX2, GBU4-5, α-enolase-1, Lmyc2;
(xxvi) p53, SSX1, KOC, CAGE, HuD, p16, GBU4-5; and
(xxvii) p53, SSX1, KOC, CAGE, SOX2, ALDH1, GBU4-5, Lmyc2.

34. The kit of claim 24 for the detection of lung cancer.

35. The method of claim 1, wherein the tumour marker antigen is a naturally occurring protein or polypeptide, a recombinant protein or polypeptide, a synthetic protein or polypeptide, a synthetic peptide, a peptide mimetic, a polysaccharide or a nucleic acid.

36. The method of claim 1, wherein the bodily fluid is selected from the group consisting of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears and wound drainage fluid.

37. A method of detecting lung cancer in a mammalian subject by detecting an autoantibody in a test sample comprising a bodily fluid from the mammalian subject, wherein the autoantibody is immunologically specific for a tumour marker antigen selected from the group consisting of p53, SSX1, SOX2, GBU4-5, HuD, p53-95 and CK8, and wherein the method comprises the steps of:
(a) contacting the test sample with a tumour marker antigen selected from the group consisting of p53, SSX1, SOX2, GBU4-5, HuD, p53-95 and CK8; and
(b) determining the presence or absence of complexes of the tumour marker antigen bound to autoantibodies present in the test sample,
wherein the presence of said complexes is indicative of the presence of lung cancer.

38. The method of claim 37, wherein two, three, four, five, six, seven or more autoantibodies are detected, and the method comprises the step of
(a) contacting the test sample with a panel of at least two or more tumour marker antigens wherein at least two of the tumour marker antigens are selected from the group consisting of p53, SSX1, SOX2, GBU4-5, HuD, p53-95 and CK8, wherein the presence of complexes containing at least two of the tumour marker antigens selected from the group consisting of p53, SSX1, SOX2, GBU4-5, HuD, p53- and CK8 is indicative of the presence of lung cancer.

39. The method of claim 37, wherein seven or more autoantibodies are detected, and the method comprises the step of (a) contacting the test sample with a panel of seven or more tumour marker antigens of which seven of the tumour marker antigens are p53, SSX1, SOX2, GBU4-5, HuD, p53-95 and CK8,
wherein the presence of complexes containing at least one tumour marker antigens selected from the group consisting of p53, SSX1, SOX2, GBU4-5, HuD, p53-95 and CK8 is indicative of the presence of lung cancer.

40. The method of claim 39, wherein the presence of complexes containing at least p53, SSX1, SOX2, GBU4-5, HuD, p53-95 and CK8 is indicative of the presence of lung cancer.

41. A kit for the detection of autoantibodies in a test sample comprising a bodily fluid from a mammalian subject comprising:
(a) a panel of two or more tumour marker antigens of which at least two of the tumour marker antigens are selected from the group consisting of p53, SSX1, SOX2, GBU4-5, HuD, p53-95 and CK8; and
(b) a reagent capable of detecting complexes of the tumour marker antigens bound to autoantibodies present in the test sample.

42. A kit for the detection of autoantibodies in a test sample comprising a bodily fluid from a mammalian subject comprising:
(a) a panel of seven or more tumour marker antigens of which seven of the tumour marker antigens are p53, SSX1, SOX2, GBU4-5, HuD, p53-95 and CK8; and
(b) a reagent capable of detecting complexes of the tumour marker antigens bound to autoantibodies present in the test sample.

43. (canceled)


SEQUENCE LISTING (26 sequences)

SEQ ID NO: 1 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 2 - protein, 631 aa, Homo sapiens
MSHWAPEWKRAEANPRDLGASWDVRGSRGSGWSGPFGHQGPRAAGSREPPLCFKIKNNMVGVVIGYSGSKIKDLQHSTNTKIQIINGESEAKVRIFGNREMKAKAKAAIETLIRKQESYNSESSVDNAASQTPIGRNLGRNDIVGEAEPLSNWDRIRAAVVECEKRKWADLPPVKKNFYIESKATSCMSEMQVINWRKENFNITCDDLKSGEKRLIPKPTCRFKDAFQQYPDLLKSIIRVGILKPTPIQSQAWPIILQGIDLIVVAQTGTGKTLSYLMPGFIHLDSQPISREQRNGPGMLVLTPTRELALHVEAECSKYSYKGLKSICIYGGRNRNGQIEDISKGVDIIIATPGRLNDLQMNNSVNLRSITYLVIDEADKMLDMEFEPQIRKILLDVRPDRQTVMTSATWPDTVRQLALSYLKDPMIVYVGNLNLVAVNTVKQNIIVTTEKEKRALTQEFVENMSPNDKVIMFVSQKHIADDLSSDFNIQGISAESLHGNSEQSDQERAVEDFKSGNIKILITTDIVSRGLDLNDVTHVYNYDFPRNIDVYVHRVGYIGRTGKTGTSVTLITQRDSKMAGELIKILDRANQSVPEDLVVMAEQYKLNQQKRHRETRSREPGQRRKEFYFLS

SEQ ID NO: 3 - protein, 424 aa, Homo sapiens
MDFSRRSFHRSLSSSLQAPVVSTVGMQRLGTTPSVYGGAGGRGIRISNSRHTVNYGSDLTGGGDLFVGNEKMAMQNLNDRLASYLEKVRTLEQSNSKLEVQIKQWYETNAPRAGRDYSAYYRQIEELRSQIKDAQLQNARCVLQIDNAKLAAEDFRLKYETERGIRLTVEADLQGLNKVFDDLTLHKTDLEIQIEELNKDLALLKKEHQEEVDGLHKHLGNTVNVEVDAAPGLNLGVIMNEMRQKYEVMAQKNLQEAKEQFERQTAVLQQQVTVNTEELKGTEVQLTELRRTSQSLEIELQSHLSMKESLEHTLEETKARYSSQLANLQSLLSSLEAQLMQIRSNMERQNNEYHILLDIKTRLEQEIATYRRLLEGEDVKTTEYQLSTLEERDIKKTRKIKTVVQEVVDGKVVSSEVKEVEENI

SEQ ID NO: 4 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 5 - protein, 602 aa, Homo sapiens
EEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYG

SEQ ID NO: 6 - protein, 176 aa, Homo sapiens
CGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQ

SEQ ID NO: 7 - protein, 355 aa, Homo sapiens
MRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQG

SEQ ID NO: 8 - protein, 378 aa, Homo sapiens
EEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITG

SEQ ID NO: 9 - protein, 354 aa, Homo sapiens
MRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQ

SEQ ID NO: 10 - protein, 336 aa, Homo sapiens
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYG

SEQ ID NO: 11 - protein, 374 aa, Homo sapiens
MLQLLVLKIEDPGCFWVIIKGCSPFLDHDVDYQKLNSAMNDFYNSTCQDIEIKPLTLEEGQVCVVYCEELKCWCRAIVKSITSSADQYLAECFLVDFAKNIPVKSKNIRVVVESFMQLPYRAKKFSLYCTKPVTLHIDFCRDSTDIVPAKKWDNAAIQYFQNLLKATTQVEARLCAVEEDTFEVYLYVTIKDEKVCVNDDLVAKNYACYMSPTKNKNLDYLEKPRLNIKSAPSFNKLNPALTLWPMFLQGKDVQGMEDSHGVNFPAQSLQHTWCKGIVGDLRPTATAQDKAVKCNMDSLRDSPKDKSEKKHHCISLKDTNKRVESSVYWPAKRGITIYADPDVPEASALSQKSNEKPLRLTEKKEYDEKNSCVK

SEQ ID NO: 12 - protein, 366 aa, Homo sapiens
MVMIISTMEPQVSNGPTSNTSNGPSSNNRNCPSPMQTGATTDDSKTNLIVNYLPQNMTQEEFRSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVGQVTGVSRGVGFIRFDKRIEAEEAIKGLNGQKPSGATEPITVKFANNPSQKSSQALLSQLYQSPNRRYPGPLHHQAQRFRLDNLLNMAYGVKRFSPITIDGMTSLVGMNIPGHTGTGWCIFVYNLSPDSDESVLWQLFGPFGAVNNVKVIRDFNTNKCKGFGFVTMTNYDEAAMAITSLNGYRLGDRVLQVSFKTNKAHKS

SEQ ID NO: 13 - protein, 188 aa, Homo sapiens [sequence omitted]

SEQ ID NO: 14 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 15 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 16 - protein, 108 aa, Homo sapiens
IQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 17 - protein, 393 aa, Homo sapiens [sequence omitted]

SEQ ID NO: 18 - protein, 95 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSS

SEQ ID NO: 19 - protein, 599 aa, Homo sapiens
MMNKLYIGNLSPAVTADDLRQLFGDRKLPLAGQVLLKSGYAFVDYPDQNWAIRAIETLSGKVELHGKIMEVDYSVSKKLRSRKIQIRNIPPHLQWEVLDGLLAQYGTVENVEQVNTDTETAVVNVTYATREEAKIAMEKLSGHQFENYSFKISYIPDEEVSSPSPPQRAQRGDHSSREQGHAPGGTSQARQIDFPLRILVPTQFVGAIIGKEGLTIKNITKQTQSRVDIHRKENSGAAEKPVTIHATPEGTSEACRMILEIMQKEADETKLAEEIPLKILAHNGLVGRLIGKEGRNLKKIEHETGTKITISSLQDLSIYNPERTITVKGTVEACASAEIEIMKKLREAFENDMLAVNQQANLIPGLNLSALGIFSTGLSVLSPPAGPRGAPPAAPYHPFTTHSGYFSSLYPHHQFGPFPHHHSYPEQEIVNLFIPTQAVGAIIGKKGAHIKQLARFAGASIKIAPAEGPDVSERMVIITGPPEAQFKAQGRIFGKLKEENFFNPKEEVKLEAHIRVPSSTAGRVIGKGGKTVNELQNLTSAEVIVPRDQTPDENEEVIVRIIGHFFASQTAQRKIREIVQQVKQQEQKYPQGVASQRSK

SEQ ID NO: 20 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 21 - protein, 188 aa, Homo sapiens
MNGDDTFAKRPRDDAKASEKRSKAFDDIATYFSKKEWKKMKYSEKISYVYMKRNYKAMTKLGFKVTLPPFMCNKQATDFQGNDFDNDHNRRIQVEHPQMTFGRLHRIIPKIMPKKPAEDENDSKGVSEASGPQNDGKQLHPPGKANISEKINKRSGPKRGKHAWTHRLRERKQLVIYEEISDPEEDDE

SEQ ID NO: 22 - protein, 129 aa, Homo sapiens
MKLKVTVNGTAYDVDVDVDKSHENPMGTILFGGGTGGAPAPAAGGAGAGKAGEGEIPAPLAGTVSKILVKEGDTVKAGQTVLVLEAMKMETEINAPTDGKVEKVLVKERDAVQGGQGLIKIGDLELIEG

SEQ ID NO: 23 - protein, 173 aa, Homo sapiens
EPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 24 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLNDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 25 - protein, 579 aa, Homo sapiens
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 26 - protein, 196 aa, Homo sapiens
TSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEE